SEQUENCE LISTING (59 sequences)

SEQ ID NO: 1 - DNA, 34 bp, artificial
  synthetic oligonucleotide primer
attgtgggccctagcatttctggaagattcattc

SEQ ID NO: 2 - DNA, 31 bp, artificial
  synthetic oligonucleotide primer
aatacccgggcaattgaatgtatgtgctgat

SEQ ID NO: 3 - DNA, 33 bp, artificial
  synthetic oligonucleotide primer
aaattctgcagttagtactttagaagtgatgct

SEQ ID NO: 4 - DNA, 35 bp, artificial
  synthetic oligonucleotide primer
aaattgagctctctagttgactatattatgcattc

SEQ ID NO: 5 - DNA, 38 bp, artificial
  synthetic oligonucleotide primer
atattacgcgtcatggagtttaacttcatcattaatag

SEQ ID NO: 6 - DNA, 37 bp, artificial
  synthetic oligonucleotide primer
atattggatcctcattttttgtagtatgtgtagtgat

SEQ ID NO: 7 - DNA, 25 bp, artificial
  synthetic morpholino targeting MEC-17 mRNA
cattcaggtcgtaagggaaatccat

SEQ ID NO: 8 - DNA, 25 bp, artificial
  synthetic morpholino targeting MEC-17 mRNA
agagaaagctattttacccgttctg

SEQ ID NO: 9 - DNA, 25 bp, artificial
  synthetic negative control morpholino
cattgacgtcctaaggcaaatgcat

SEQ ID NO: 10 - DNA, 20 bp, artificial
  synthetic oligonucleotide primer
ggtcggaaagcgcatgggag

SEQ ID NO: 11 - DNA, 21 bp, artificial
  synthetic oligonucleotide primer
gaagtcgaagagctctgagcc

SEQ ID NO: 12 - DNA, 18 bp, artificial
  synthetic oligonucleotide primer
gattcgctggagatgatg

SEQ ID NO: 13 - DNA, 20 bp, artificial
  synthetic oligonucleotide primer
gtctttctgtcccataccaa

SEQ ID NO: 14 - DNA, 32 bp, artificial
  synthetic oligonucleotide primer
attatgtttaaaccaagctcgagttctccatc

SEQ ID NO: 15 - DNA, 31 bp, artificial
  synthetic oligonucleotide primer
aattatgatcacagcaaaggattcaaggctc

SEQ ID NO: 16 - DNA, 32 bp, artificial
  synthetic oligonucleotide primer
ataatgtttaaaccggcgagaagagctatcaa

SEQ ID NO: 17 - DNA, 24 bp, artificial
  synthetic oligonucleotide primer
aatttggagaactcgagcttggcc

SEQ ID NO: 18 - RNA, 19 nt, artificial
  synthetic siRNA targeting MEC-17
guagcuaggucccgauaua

SEQ ID NO: 19 - RNA, 19 nt, artificial
  synthetic siRNA targeting MEC-17
gaguauagcuagaucccuu

SEQ ID NO: 20 - RNA, 19 nt, artificial
  synthetic siRNA targeting MEC-17
gggaaacucaccagaacga

SEQ ID NO: 21 - RNA, 19 nt, artificial
  synthetic siRNA targeting MEC-17
cuugugagauugucgagau

SEQ ID NO: 22 - DNA, 23 bp, artificial
  synthetic negative control siRNA
gcugacccugaaguucaucugtt

SEQ ID NO: 23 - DNA, 34 bp, artificial
  synthetic oligonucleotide primer
aaattgagctctggagttcccgttcgatgtggat

SEQ ID NO: 24 - DNA, 39 bp, artificial
  synthetic oligonucleotide primer
aatagaattcccgcggactaagctttggccatggttacc

SEQ ID NO: 25 - protein, 191 aa, Drosophila melanogaster
MVEFRFDIKPLFAQPIIKVTSNLLPNTFRGDRRQCLDATSKMTEIIDQLGQLSATSQGLSKPVTTAQRLRMSDNQTIYLLADNEAGHNGAVLGLLKVGTKNLYLFDEAGKTRMVEQTPSILDFYVHESRQRAGLGKRLFQTMLNEEQWTARKCSVDRPSEKLLSFLSKHYGLKRIIPQANNFVLYEGFFND

SEQ ID NO: 26 - protein, 198 aa, Drosophila melanogaster
MVEFAFDIKHLFPQSIIRVQAHSLRPKVTQCRRYAQTERGKSTMTSCRLSEILNIMGKLSADAQGLCHAVTTAQRLRMSDNQTIYLLADNEAGHNGAVLGLLKVGTKNLYLFDEAGKTRMVEQTPSILDFYVHESRQRAGLGKRLFQTMLNEEQWTARKCSVDRPSEKLLSFLSKHYGLKRIIPQANNFVLYEGFFND

SEQ ID NO: 27 - protein, 188 aa, Trypanosome brucei
MTHNVMCDDVLPQLNLPDGVTRWNANLLEEERRLRNSDGHADRIILTINTLGKRSKEAQSLNTILTSVPRLRENRDARLYLLCHGGRGVGILKIGVKRLFVVPPSHAGLMEIEPVCVLDFFVDTSNQRQGYGKILFEHMLAFERLSPGDVAIDRPSVKFLAFLRKHYGLVEYTPQSNNFVVFHKYFER

SEQ ID NO: 28 - protein, 187 aa, Trypanosoma cruzi
MSSTSQVALLPKLSLPDGVTVWDGTALEYERRCNNVDEHAVHLMQTINILGIRSKEAQCLNTVLTSVARLRENRQARVYLLCQDGYGVGILKMGVKKLFVTHPSYSSLVEIDPLCVLDFFVDTSFQRKGFGKTLFDAMLLNEGLNPGEVAIDRPSVKFLAFLQKYYGLVEYTPQSNNFVVFHRYFDK

SEQ ID NO: 29 - protein, 188 aa, Xenopus tropicalis
MEFDFDVHKIFLEPITKLDSSLIPSRRPLIASSEAQKQIMTVIDEIGKASAKAQRLPAPITSASRMQTNKHHLYILKDCTPKTAGRGAVIGFLKVGCKKLFVLDQKGSHIEAEPLCILDFYIHETLQRHGFGKELFTFMLKNEQVDVHHLAIDRPSEKFLSFLRKHFNLWSTIPQVNNFVVFDGFFRD

SEQ ID NO: 30 - protein, 188 aa, Xenopus laevis
MEFEFDVHKIFLEPITKLDNNLIPPRRPLISSSEAQKQIMTVIDEIGKASAKAQRLPASITSASRMQANKHHLYILKDCTPKTAGRGAVIGFLKVGYKKLFILDQKGSHIEAEPLCILDFYIHESLQRHGFGKELFSFMLRNEQVDVQHLAIDRPSEKFLSFLRKHFNLWSTIPQVNNFVVFEGFFRD

SEQ ID NO: 31 - protein, 192 aa, Homo sapiens
MEFPFDVDALFPERITVLDQHLRPPARRPGTTTPARVDLQQQIMTIIDELGKASAKAQNLSAPITSASRMQSNRHVVYILKDSSARPAGKGAIIGFIKVGYKKLFVLDDREAHNEVEPLCILDFYIHESVQRHGHGRELFQYMLQKERVEPHQLAIDRPSQKLLKFLNKHYNLETTVPQVNNFVIFEGFFAH

SEQ ID NO: 32 - protein, 186 aa, Danio rerio
MDFPYDLNALFPERISVLDSNLSAGRKAHGRPDPLPQVTTVIDELGKASSKAQQLPAPITSAAKLQANRHHLYLLKDGEQNGGRGVIVGFLKVGYKKLFLLDQRGAHLETEPLCVLDFYVTETLQRHGYGSELFDFMLKHKQVEPAQMAYDRPSPKFLSFLEKRYDLRNSVPQVNNFVVFAGFFQS

SEQ ID NO: 33 - protein, 185 aa, Ciona intestinalis
MEFDFNINHLFPDKITLVGENSSYRKHANSKILQRNLQIVIEVLGQRSARAQQLTGSITTLLKSQLNNQRIYVLKEANANNGLGCVIGFLKTGKKRLFVLDRDGNHNEMNPLCVLDFYVHESQQRKGCGLCLFKHMLHVEGVKASHLAIDRPSHKFISFLKKHFSLWATVPQVNNFVIFDGFFKN

SEQ ID NO: 34 - protein, 180 aa, Tetrahymena thermophila
MEFNFIINKLVQLDQQGLGVYIPRASRSKVSSQQEQQLGQVLNTMGERSAIAQGLKQVITNYDKVQGTDQRVYIVAEGRTCQGFLKVGQKNLFYRDMMGNIKEIKPLCVLDFYVHESCQRQGYGKLLFEYMIQCEQTSPEKLAYDRPSPKLIAFLKKHYNLVKYIAQNNNFVVFDQYFRS

SEQ ID NO: 35 - protein, 174 aa, Paramecium tetraurelia
MQFQFPLQKALQTSQNGISVISASNSRRNCYLDEVIDRMGEASAIAQQLKQIITTASKFYGSDQRIYLKADGKNCLGLLKVGKKNLFYRDYSGSIKEMQPLCVLDFYVHESVQRMGVGKELFEEMLKSEQIKPEKLAYDRPSQKLIGFLNKHYNLNQYVPQNNNFVIFNQYFGQ

SEQ ID NO: 36 - protein, 179 aa, Caenorhabditis elegans
MQVDADLRPILGPQLVRLDPMRVKQLQDPIVYEAIDNLAKLSAHCLQLRTPLTTCEKLINSDSTLYLSWKYDEEEKVSRLMGFAKVGRKKLFLYDSQMQTYEGEILCLLDFYVHFSCQRQGVGQQILDYMFSQEHTEPYQLALDNPSVTLLGFMSQKYGLIKPVWQNTNFVVFEELFLA

SEQ ID NO: 37 - protein, 179 aa, Giardia lamblia
MQFGCNVAEAFGLRRSGVVLLTDQSLRSMPLSQQKKVEIILDGMGRGSQAAQGLPSPITSLAFIRDSHHFLFLAVDEDQCLGILKGGIKHLFMLDSQNETHEMDAMCCLDFYTHETVQRRGIGTRLFRAMELHTHISAQGWAFDRPSPKLLAFLSKVYDMHDFKAQPNNFLMLDASIRL

SEQ ID NO: 38 - protein, 183 aa, Caenorhabditis elegans
MEIAFDLSTIFTDNIQRLTRTDLLKYGPKRYWAVAQSIDCLGEMSSKFHGWKRVITMYDKIVDHDEEQTTYIMWEKVNGSKSILKGLLRVGYKTLYLTDNEQNQYMEKAMCILDFFVVPTEQRSGNGFKMFDEMLKAENVTVDQCAFDKPSAALQQFLEKYYDRKDLVWQSNKYALCSNFFIG

SEQ ID NO: 39 - DNA, 21 bp, artificial
  synthetic oligonucleotide primer
acgataaggtataaggaacag

SEQ ID NO: 40 - DNA, 21 bp, artificial
  synthetic oligonucleotide primer
aagttatctatcttatccagg

SEQ ID NO: 41 - protein, 262 aa, Caenorhabditis elegans
MQVDADLRPILGPQLVRLDPMRVKQLQDPIVYEAIDNLAKLSAHCLQLRTPLTTCEKLINSDSTLYLSWKYDEEEKVSRLMGFAKVGRKKLFLYDSQMQTYEGEILCLLDFYVHFSCQRQGVGQQILDYMFSQEHTEPYQLALDNPSVTLLGFMSQKYGLIKPVWQNTNFVVFEELFLALSAENGIEKPPPDGWRRPMTPRRLGTGMTDTRWLQHAVSGHQSKGNAMAAPVDADMTPQGALSNRAHQAKARKAHILSSKPLW

SEQ ID NO: 42 - protein, 263 aa, Caenorhabditis elegans
MEIAFDLSTIFTDNIQRLTRTDLLKYGPKRYWAVAQSIDCLGEMSSKFHGWKRVITMYDKIVDHDEEQTTYIMWEKVNGSKSILKGLLRVGYKTLYLTDNEQNQYMEKAMCILDFFVVPTEQRSGNGFKMFDEMLKAENVTVDQCAFDKPSAALQQFLEKYYDRKDLVWQSNKYALCSNFFIGRHPTVPFTPRQTKRASRASSAVSSHASSRNTSPIGRNRPRHDSVADLMRQDMLAGVRAEVDPNSPTGLKNARDFGHRRIW

SEQ ID NO: 43 - protein, 410 aa, Ciona intestinalis
MEFDFNINHLFPDKITLVGENSSYRKHANSKILQRNLQIVIEVLGQRSARAQQLTGSITTLLKSQLNNQRIYVLKEANANNGLGCVIGFLKTGKKRLFVLDRDGNHNEMNPLCVLDFYVHESQQRKGCGLCLFKHMLHVEGVKASHLAIDRPSHKFISFLKKHFSLWATVPQVNNFVIFDGFFKNRSDTVRNNNAKSEWNNRQFRPLSRAVSDDIIKRNRFSHDVPNKVNGLPPLPPNRRDRYGDNDVVTNPYRRNSTNQNGHVKGDVLQGSHMADKKDIGEERAKDNALPGIATNQLEESRVQTNPQEVTENAYNGMNKHSSPVDQLKCNSLSQDQKSSPKIARKPATPPHLKPSSIIDELKAKDAAYLSNRNLSTQNQSRLWYQRQLQGTRSSWNVLGVPTRPFWNAQ

SEQ ID NO: 44 - protein, 461 aa, Drosophila melanogaster
MVEFRFDIKPLFAQPIIKVTSNLLPNTFRGDRRQCLDATSKMTEIIDQLGQLSATSQGLSKPVTTAQRLRMSDNQTIYLLADNEAGHNGAVLGLLKVGTKNLYLFDEAGKTRMVEQTPSILDFYVHESRQRAGLGKRLFQTMLNEEQWTARKCSVDRPSEKLLSFLSKHYGLKRIIPQANNFVLYEGFFNDGESGNGGGNGHANGTPNGLHITNSPNTHLFGATYLGEDSNQRRGSQQQTTPNARLQQITQISPSGRYGAKRPTCSMAEIIHAGNSKGGNGNGSAEANSGNGNHDIPEIAEQLQRQSLADLEANSYEPEPEVEPEPEPEPEPEPEPEVITPPSPPPKSHTPTPPSVRSPEVAESIVGDNRRAPAFQLSKQHTGMKNRSFGVGMAVMPSSKMEFDQMEREDFGVVKINRPPGHEVTSPGQDNTDAMSTVSSGGGGLTDQGYYDLKFYHNKLW

SEQ ID NO: 45 - protein, 287 aa, Drosophila melanogaster
MVEFAFDIKHLFPQSIIRVQAHSLRPKVTQCRRYAQTERGKSTMTSCRLSEILNIMGKLSADAQGLCHAVTSADKLASDQVVYLMADKAAGHWEITGLLKVGTKDLFVFDQGGCYRRLNQTPAILDFYVHESRQRCGQGKLLFEWMLEKQGWSAHKCTVDRPSNKMLAFMAKHYGLVRTIPQGNNFVLYEGFFDDPITTCKSASGLQATGSGCRSRSQGHYVRQEQDQAQIKHGQANRNTVQNDANSGPFRQDQKIVVGTSIYRRRWKSPRTLARAGCREVSGGRRF

SEQ ID NO: 46 - protein, 297 aa, Danio rerio
MDFPYDLNALFPERISVLDSNLSAGRKAHGRPDPLPQVTTVIDELGKASSKAQQLPAPITSAAKLQANRHHLYLLKDGEQNGGRGVIVGFLKVGYKKLFLLDQRGAHLETEPLCVLDFYVTETLQRHGYGSELFDFMLKHKQVEPAQMAYDRPSPKFLSFLEKRYDLRNSVPQVNNFVVFAGFFQSRSAVQLRKVPPRKPEGEIKPYSLMEREVVREEQRVLPWPFVRPGGPPHSPPLLPSSPQSRSLSVGSSPSRAPLRPAAATVLQQGQTPSSPLNDSCRAKRTSSLNRSRLSFH

SEQ ID NO: 47 - protein, 362 aa, Giardia lamblia
MQFGCNVAEAFGLRRSGVVLLTDQSLRSMPLSQQKKVEIILDGMGRGSQAAQGLPSPITSLAFIRDSHHFLFLAVDEDQCLGILKGGIKHLFMLDSQNETHEMDAMCCLDFYTHETVQRRGIGTRLFRAMELHTHISAQGWAFDRPSPKLLAFLSKVYDMHDFKAQPNNFLMLDASIRLWGAEFKQYRRSKKHYIPDAYLLPETRESEYLGEAELTKRTLIRKSTAVIPQTKTTQSEDAPARALTADELLSKRSVVLPTASRTPSLPDQPQSVAAAYMNKRIEGAGPSFEQYMRDHYGAKSLIVPSEIQTSLNHSKDSVSQEDMIQRQRQLDRMAFTLAREANARGSIHNTVGRGVICGRRG

SEQ ID NO: 48 - protein, 323 aa, Homo sapiens
MEFPFDVDALFPERITVLDQHLRPPARRPGTTTPARVDLQQQIMTIIDELGKASAKAQNLSAPITSASRMQSNRHVVYILKDSSARPAGKGAIIGFIKVGYKKLFVLDDREAHNEVEPLCILDFYIHESVQRHGHGRELFQYMLQKERVEPHQLAIDRPSQKLLKFLNKHYNLETTVPQVNNFVIFEGFFAHQHRPPAPSLRATRHSRAAAVDPTPAAPARKLPPKRAEGDIKPYSSSDREFLKVAVEPPWPLNRAPRRATPPAHPPPRSSSLGNSPERGPLRPFVPEQELLRSLRLCPPHPTARLLLAADPGGSPAQRRRTR

SEQ ID NO: 49 - protein, 184 aa, Micromonas sp. RCC299
MQHPRGVAGLTPGGVSVWTRDAIVRLPPDEYRAISALIDEAGARSARAQGLPAPITSTDRLLEDQRLYLACSDATRPRGGPSVLVGILKVGPKRLFVAKPSGGMEEMEPCCVLDFYVHESSQRGGWGSLLFDAFLQREDRHPARLAYDRPSPKLVAFMAKHHNLRAFAKQNNNFVVFDEYWSEE

SEQ ID NO: 50 - protein, 564 aa, Physcomitrella patens
MVEFVCSIPSLKTFTEVPKITAWNSYQLMNLMRGKDGEEMQIIINYMGELSAIAQGLRGPITSVDRLLQSFQKIYLLTSSVPGNSGHILAQGILKVGQKHLFIRRTPNAPLVEISPLCVLDFYAGTGAYVIQVHARAGEEEAMSAWVRLSSFPSPVPVPVEITSNYDKPSNKLLSFMSKYYNLRSYNDQANQFVVYNDYFVDAKNEAQKEKENRDKELNGTPDIGTNPRKDEVLHDSQANLQVIDVDDQCSSNRSDISRLTTAEARPPNLHVKEPPKQPLFRGGRKKIWQSKQGYSEKPPNSADTLHTTDARSSLQSPTKATLSLKDAYRQGLNDQGISLHARKSSNSLSSEPKRCQENHGANNLHEPGVTLLYSVIGEEERLKIQSMCTPSLAQKMNVKHPTRANHKDPKPIKPRPNMGSPVTRPTRQLTQKDPPPSCPLWADAKELSESQHWQERNPVDYKRTGKTMPVRNTRAEKIRSSINRMGDARNLAKALVHLSEQSGWFHCQWINDICDTWCVPHYLIKMHSTVNWSSQSWKNRHAYTELAVTGLLANGHYSQMGSA

SEQ ID NO: 51 - protein, 249 aa, Paramecium tetraurelia
MQFQFPLQKALQTSQNGISVISASNSRRNCYLDEVIDRMGEASAIAQQLKQIITTASKFYGSDQRIYLKADGKNCLGLLKVGKKNLFYRDYSGSIKEMQPLCVLDFYVHESVQRMGVGKELFEEMLKSEQIKPEKLAYDRPSQKLIGFLNKHYNLNQYVPQNNNFVIFNQYFGQGTQPIAQGRSQKYSRNSQIDQLDIMVQQISQNKTNQQQLPQQKLGYQMSTPWAIDNHTNIYNNINSQRTNVYKIN

SEQ ID NO: 52 - protein, 267 aa, Schistosoma japonicum
MDFRAGLENVLQQEVTVIHGEETRKLCIANNKYLNGKDADTFRNLAVLLDHLGERSAKAQKLPKPVTSFIKFRNSDQSIFLLSDIPVKKFVILIYMFFRVLGFLKVGRKRLFVHDSKGVCVECIPLCILDFYIHESHQRKGYGKKLFDFMLKTENIQPSYLAIDLPSMKMIQFLHKHYHLINPIYSPNNFVVYSEFFNNLNNSNYSIVQSKLTTTNCFLKSNSSRIHQNKHNHSIVSNNNNNNNNNIIIIIIIQNITHHNNQLTIEQ

SEQ ID NO: 53 - protein, 334 aa, Trypanosoma brucei
MTHNVMCDDVLPQLNLPDGVTRWNANLLEEERRLRNSDGHADRIILTINTLGKRSKEAQSLNTILTSVPRLRENRDARLYLLCHGGRGVGILKIGVKRLFVVPPSHAGLMEIEPVCVLDFFVDTSNQRQGYGKILFEHMLAFERLSPGDVAIDRPSVKFLAFLRKHYGLVEYTPQSNNFVVFHKYFERHQQQRRGVGGSGRSGYQHCNETTTQQLGTQSGLLEDINQTHPAPSYALRGVVMGHTGPPLDLTNVTQQQKPYHQPFATGRKTSYELQYERYLQSQNCRPTGNAGYGGGNGPASSAEVRATNCQARRRTSPTRSGVPYNIINGSTGS

SEQ ID NO: 54 - protein, 328 aa, Trypanosoma cruzi
MSSTSQVALLPKLSLPDGVTVWDGTALEYERRCNNIDEHAVHLMQTINILGIRSKEAQCLNTVLTSVARLRENRQARVYLLCQDGYGVGILKMGVKKLFVTHPSYSSLVEIDPLCVLDFFVDTSFQRKGFGKTLFDAMLLNEGLNPGEVAIDRPSVKFLAFLRKYYGLVEYTPQSNNFVVFHRYFDKWQPQRGKGHRGGNAVPTRSIVRPQNCLRVYPEYQSTTGPNDNFEEDATHRTPPPPLPPPLVPQGSVNSPGPGKKTAYELQYEEYLREQAYRRRQGGDPRLQPVPNPVSSSEIAAASCGARRRMSPTRSGVQYNIISGTPEH

SEQ ID NO: 55 - protein, 359 aa, Tribolium castaneum
MEFKFAVNDVFKQPIVKIGNNLLPPGFTGDRRAFWDVVGKVSEVVNAMGEASAAAQGLTKPITTAERLRNSEHSVYLLIDQNAGNGRGAVTGMLKTGMKGLYVFDRNGQHYQVSPPCVLDFYVHESRQRTGLGKRLFEHMLQTESIEPVKMAIDRPSEKFLGFLNKHYSLNNPVKQMNNYVVFDGFFPGAQDKNGTVESHSSHSNTPVGKKQSANGLQTSTYASPLGRYGAPRPPCSMGQIIHNQTSTIAKTPEPTGETQVMDEKVNSSPKLERPRSLSIQPSDEPTEDNNQNENDVESEVLEEVEIIENLTIENGADVVDTKNEKIATPEAKTPGKRTPSHLTEQGFFDLKFYHNKLW

SEQ ID NO: 56 - protein, 488 aa, Tetrahymena thermophila
MFKNQLLSLSLRIQKLRLCRQGNQLKVLQKSINQLLFKTQSIDQILQYLQQKKKTHQSILENQNQHIHSIGLVSQLTNQKINKQKNQKAMEFNFIINKLVQLDQQGLGVYIPRASRSKVSSQQEQQLGQVLNTMGERSAIAQGLKQVITNYDKVQGTDQRVYIVAEGRTCQGFLKVGQKNLFYRDMMGNIKEIKPLCVLDFYVHESCQRQGYGKLLFEYMIQCEQTSPEKLAYDRPSPKLIAFLKKHYNLVKYIAQNNNFVVFDQYFRSDASSQNKQNQNTRSYSQPYSDYSSQIPTNYPQQQQQQSNSKSYPYKQENNIDLMQHSSSRNNKEFLNAGRAILSKEEIYKKDNLSQQNIENTLNNINNSQYSTKSQQQQQYQKDYQLDKYENNWGADKNIKKPPFPGQDRQLDKIQQKIQQTERELDVVNQQIIKQRQNLSQDPLTQNHRAQNVYNTNQFGTSPWAQTGFNYYSTSSSNYGNHYTYYKK

SEQ ID NO: 57 - protein, 418 aa, Xenopus laevis
MEFEFDVHKIFLEPITKLDNNLIPPRRPLISSSEAQKQIMTVIDEIGKASAKAQRLPASITSASRMQANKHHLYILKDCTPKTAGRGAVIGFLKVGYKKLFILDQKGSHIEAEPLCILDFYIHESLQRHGFGKELFSFMLRNEQVDVQHLAIDRPSEKFLSFLRKHFNLWSTIPQVNNFVVFEGFFRDRKASVKKTPAKRTEGEIKPYSLTDRDFLKQEEGLPWPLSQAQINLNRASSLGSSPTRACSRPPPGEEDFVKSLRNCRPHSLQRAASSEQEDHSQRRRTSEMNLSRGLLAQKNGYSRYLSPPPPLLTQGPYAAAQIKEQQSRTDSSAQEGRTQDRPNGSNSQHQNDLISSKQHVQDLHMELAAGRTMSDLKEGQNATKSPWCDHPSYTVLGTVLNAAWVKKKQELRSTRPW

SEQ ID NO: 58 - protein, 297 aa, Xenopus (Silurana) tropicalis
MEFDFDVHKIFLEPITKLDSSLIPSRRPLIASSEAQKQIMTVIDEIGKASAKAQRLPAPITSASRMQTNKHHLYILKDCTPKTAGRGAVIGFLKVGCKKLFVLDQKGSHIEAEPLCILDFYIHETLQRHGFGKELFTFMLKNEQVDVHHLAIDRPSEKFLSFLRKHFNLWSTIPQVNNFVVFDGFFRDWKASVKKTPAKRTEGEIKPYSLTDRDFLKQEEGLPWPFSQSQLNLNRASSLGSSPTRACSRHSPGEEDFVKSLRNCRPHSLHRTANSEQEDHSQRRRTSSLNRPQSIHH

SEQ ID NO: 59 - protein, 639 aa, Chlamydomonas reinhardtii
MEFDFASLAFGGEQHISFWDSKRIATLKPDDQELLTKLLDVFGKKSAVAQGLGAPVTDIYRLRSTDQRLYLYMYRESRKTVVLGGLKVGSKRLFVRTGTADLREIEPVCVLDFYVHESCQRQGVGKALFEHFLMAEGHDPATLAYDRPSPKLLAFLRKHYGLEQYVPQSNNYVVFDRYWELCPPGSRQPHRGPGSMPPGASGQLQGRNASRGSVASISTPSGHGGHGPRATPLMGMMPPGSGAGGYPPHWTPSALGSPSGGHPGGPPAPIGNSPSFGRRWASNNFGQGPPQAAPPYPAPGTAGGPAFSIPPAGGDAQGGIVDALDAFARQQQQQGSGASSPQRGGAGAPWEGGAAGPAGTGASGFGGGAGTWPPQGAPELPPAGAPSGMQPPGSRGAYSYRPPWATDEAAGPSGGSASGGVDAGPGAGPGAAGYAGFNSPPPPMRTPSKDRFGMGPGNGGPAPVQRSPMHSILGGGGGYDAVAGGRSGAAQTRALQQQLLSMQAGDGAVAAAAAGGPVLGGGYGGGGGHPGSYQQQQQHAPPSLYSGAPPAHGAGQGGDEDGGRLPGMMGHGSLALGMLGAAPSGRHGAGSGGHAGGPGGGPPGGTSAGTPPAAGGHSAAKLMAVKQRSGAGAADCLVW

(19) United States
(12) Patent Application Publication
(10) Pub. No.: US 2014/0248635 A1
(43) Pub. Date: Sep. 4, 2014

(54) ALPHA-TUBULIN ACETYLTRANSFERASE

(71) Applicant: University of Georgia Research Foundation, Inc., Athens, GA (US)

(72) Inventors: Jacek Gaertig, Athens, GA (US); Dorota Wloga, Legionowo (PL); Shilpa Akella, Athens, GA (US)

(73) Assignee: UNIVERSITY OF GEORGIA RESEARCH FOUNDATION, INC., Athens, GA (US)

(21) Appl. No.: 14/172,226

(22) Filed: Feb. 4, 2014

Related U.S. Application Data
(63) Continuation of application No. 13/653,905, filed on Oct. 17, 2012, now abandoned.
Application No. 13/653,905 is a continuation-in-part of application No. PCT/US2011/033063, filed on Apr. 19, 2011.
(60) Provisional application No. 61/327,462, filed on Apr. 23, 2010.
(60) Provisional application No. 61/325,680, filed on Apr. 19, 2010.

(51) Int. Cl. C12Q 1/48; G01N 33/50

(52) U.S. Cl. CPC C12Q 1/48; G01N 33/502
USPC 435/7.4; 435/15

(57) ABSTRACT

Polypeptides with tubulin acetyltransferase activity are described, as are nucleic acids encoding said polypeptides, and methods of use. The invention further provides enhancers and inhibitors of tubulin acetyltransferase activity, as well as cells having altered tubulin transferase activity.

### CONTINUING APPLICATION DATA

This application is a continuation of U.S. patent application Ser. No. 13/653,905, filed Oct. 17, 2012, which is a continuation-in-part of International Application Serial No. PCT/US2011/033063, with an International Filing Date of Apr. 19, 2011, published as WO2011/133559 on Oct. 27, 2011, which in turn claims the benefit of U.S. Provisional Application Ser. No. 61/325,680, filed Apr. 19, 2010, and U.S. Provisional Application Ser. No. 61/327,462, filed Apr. 23, 2010, each of which is incorporated by reference herein.

### GOVERNMENT FUNDING

This invention was made with government support under Grant No. MBC-033965 awarded by the National Science Foundation; and Grant Nos. R01GM089912, R01GM074212, and R01AI067981 awarded by the National Institutes of Health. The government has certain rights in the invention.

### SEQUENCE LISTING

This application contains a Sequence Listing electronically submitted to the United States Patent and Trademark Office via EFS-Web as an ASCII text file entitled “235-01330121_SequenceLising.txt” having a size of 87 bytes and created on Feb. 4, 2014. The information contained in the Sequence Listing is incorporated by reference herein.

### BACKGROUND

Microtubules are fibers made of α-tubulin and β-tubulin dimers. Microtubules form cytoplasmic networks and serve as frameworks of important organelles, including the mitotic spindle, centrioles, cilia and bundles inside neurites. The biogenesis of microtubules involves the synthesis of tubulin polypeptides, chaperonin-assisted folding and dimerization of α-tubulin and β-tubulin, transport to the sites of assembly, nucleation, polymerization, deposition of post-translational modifications (PTMs), and binding of diverse microtubule-associated proteins (MAPs).

During mitosis, the microtubule undergoes dramatic changes to transition from an interphase monopolar organization to the bipolar spindle. Thus, tubulins are a major target of anti-cancer drugs which act by disrupting the dynamic properties of MTs during mitosis and in some cases inducing apoptosis. However currently available widely used microtubule-targeting compounds (such as vinblastine or paclitaxel) suffer from a major limitation—they target microtubules indiscriminately. Paclitaxel, for example, a compound that hyperstabilizes microtubules and blocks cells in mitosis, is currently the most widely used drug to treat ovarian, breast, lung cancers and AIDS-related Kaposi's sarcoma (Ring et al. (2005) Cancer Treat Rev 31, 618-627; Cheung et al., (2005) Oncologist 10, 412-426). However, paclitaxel produces strong side effects by affecting non-mitotic microtubules, in particular in nerve cells (Hennenfent et al. (2006). Ann Oncol. 17, 735-749). Paclitaxel also affects the bone marrow leading to hematopoietic deficiencies in ˜90% of patients (Hagiwara et al. (2004) Breast Cancer 11, 82-85). Ideally, future anti-microtubule compounds should affect as few cell types as possible besides intended targets. Humans have several isotypes of α-tubulin and β-tubulin, some of which are expressed in a restricted fashion. However, tubulins are highly conserved and differ mainly in the small portion of their primary sequence near the C-terminal end (Luduena (1998) Int. Review Cytol. 178, 207-274). Thus, developing isotype-specific inhibitors for tubulin primary polypeptides is likely to be difficult.

Post-translational modifications of microtubules are ubiquitously present in eukaryotes and their physiological importance is increasingly well documented (Rosenbaum (2000) Current Biol. 10, R801-R803, Westermann et al. (2003) Nat Rev Mol Cell Biol 4, 938-947). The most studied post-translational modifications include acetylation of α-tubulin, detyrosination of α-tubulin, palmitoylation of α-tubulin, and phosphorylation, glutamylation, and glycylation of α-tubulin and β-tubulin.

Post-translational modifications are believed to function in regulating interactions of microtubules with MAPs (such as dynein and kinesin motors) (Rosenbaum (2000) Current Biol. 10, R801-R803), Westermann et al. (2003) Nat Rev Mol Cell Biol 4, 938-947). Most post-translational modifications are located on the C-terminal tails of tubulins, highly flexible acidic domains present on the surface of microtubules (Nogales et al. (1999) Cell 96, 79-88). The tails are also the major sites of interactions with kinesin and dynein motors, structural MAPs (MAP2, Tau), microtubule-severing protein katanin, and plus end-depolymerizer (MCAK) (Skiniotis et al. (2004) Embo J 23, 989-999, Ovechkina et al. (2002) J Cell Biol 159, 557-562, Lu et al. (2004) Mol Biol Cell 15, 142-150). By regulating the activity of tubulin modifying enzymes, cells can mark microtubules in specific subcellular areas to regulate binding and activity of MAPs in a localized fashion. Detyrosination of α-tubulin promotes transport of vimentin intermediate filaments mediated by kinesin-1 (Kreitzer et al. (1999) Mol. Biol. Cell 10, 1105-1118). Another post-translational modification, polyglycylation, appears to acts as a mark to regulate assembly of cilia and severing of stable cortical microtubules in Tetrahymena (Thazhath et al. (2002) Nature Cell Biol. 4, 256-259; Thazhath et al. (2004) Mol Biol Cell 15, 4136-4147). Acetylation of the ε-amino group of K40 on α-tubulin is a conserved PTM on the luminal side of microtubules (Nogales et al., 1999 Cell 96:79-88) that was discovered in the flagella of Chlamydomonas reinhardtii (L'Hernault and Rosenbaum, 1983 J. Cell Biol. 97:258-263; LeDizet and Piperno, 1987 Proc. Natl. Acad. Sci. USA 84:5720-5724). Studies on the significance of microtubule acetylation have been limited by the undefined status of the α-tubulin acetyltransferase. The basic principle of specific post-translational modifications acting alone or in combination to regulate binding of a variety of microtubule interactors is likely to be general. By analogy with the epigenetic “histone code,” eukaryotic cells appear to utilize a “microtubule code” to coordinate MAPs.

Inhibitors of a forward post-translational modification enzyme are not known in the art. Furthermore, inhibitors are known for only one of the reverse post-translational modification enzymes, tubulin deacetylase HDAC6 (related to histone deacetylases). Overexpression of HDAC6 decreased acetylation and increased chemotactic motility of mammalian cells (Hubbert et al. (2002) Nature 417, 455-458). HDAC6 can be inhibited with trichostatin A (a broad inhibitor of deacetylases) (Matsuyama et al. (2002) Embo J 21, 6820-6831), and tubacin (a specific inhibitor) (Haggarty et al. (2003) Proc. Natl. Acad. Sci. U.S.A. 100, 4389-4394). Chemically blocking HDAC6 increased the level of acetylation on microtubules and decreased cell motility as well as disrupted localization of the p58 MAP (a Golgi-microtubule linker) in vivo (Haggarty et al. (2003) Proc. Natl. Acad. Sci. U.S.A. 100, 4389-4394). Inhibitors of HDAC6 has helped to uncover potential new functions for a-tubulin acetylation, including its role in the immune synapse formation (Serrador et al. (2004) Immunity 20, 417-428), and during infection of cells by HIV (Valenzuela-Fernandez et al. (2005) Mol Biol Cell 16, 5445-5454). HDAC6 is upregulated in the acute myeloid leukemia cells (Bradbury et al. (2005) Leukemia 19, 1751-1759), and is one of the estrogen-responsive genes in breast carcinoma. Blocking HDAC6 with tubacin inhibited estradiol-induced cell migration of breast carcinoma cells (Saji et al. (2005) Oncogene 24, 4531-4539). Tubacin also increased anti-cancer effects of other compounds, including a proteasome inhibitor, bortezomid (Hideshima et al. (2005) Proc Natl Acad Sci USA 102, 8567-8572). Although HDAC6 effects could be mediated by at least two different substrates (α-tubulin and HSP90, (Kovacs et al. (2005) Mol Cell 18, 601-607), it is very likely that the effects on cell motility are microtubule-mediated. Importantly, HDAC6 is required for the synergistic inhibitory action of paclitaxel and lonafarmib (an inhibitor of farnesyltransferase) on cancer cells (Marcus et al. (2005) Cancer Res 65, 3883-3893). However, these targeting efforts are limited to one post-translational modification and specifically, to one enzyme, HDAC6 deacetylase. A general strategy for identifying post-translational modification drugs and in particular inhibitors of forward enzymes responsible for deposition of PTMs is needed.

### SUMMARY OF THE INVENTION

The present invention provides methods for affecting α-tubulin acetyl transferase (α-TAT) activity. For example, MEC-17 is a polypeptide having αTAT activity. The MEC-17 polypeptide of the present invention may be a C. elegans, a Tetrahymena, a zebrafish, or a mammalian MEC-17 polypeptide. Preferably, the mammalian MEC-17 polypeptide of the invention is encoded by the human c6orf134 locus.

In one aspect, the invention is directed to a method for affecting α-TAT activity in a cell that expresses a MEC-17 polypeptide. The method includes introducing into the cell a compound that affects the amount or activity of the MEC-17-encoding RNA transcript or the MEC-17 polypeptide. The cell may be present in an organism, in a tissue, in a fluid that has been removed from an organism, or in a cell culture. In one embodiment, the compound inhibits, reduces, or eliminates the amount of activity of the MEC-17-encoding transcript or the MEC-17 polypeptide to cause, directly or indirectly, a decrease in acetylation of an α-tubulin. Preferably, the compound causes a decrease in acetylation of a lysine at position 40 (K40) of an α-tubulin. In another embodiment of the invention, the compound increases or stimulates the amount of activity of the MEC-17-encoding transcript or the MEC-17 polypeptide. Preferably, the compound causes, directly or indirectly, n increase in acetylation of an α-tubulin. The compound may activate an MEC-17 gene, cause increased translation of an MEC-17-encoding RNA transcript, or agonize or stimulate the MEC-17 polypeptide.

In another aspect, the invention is directed to a method for diagnosing a disease, disorder, or condition or the nervous system or the immune system in a subject. The method includes detecting a mutation in a MEC-17 gene or an MEC-17 polypeptide. Preferably the mutation affects the amount of activity of an MEC-17 polypeptide. The method of the invention may be useful in diagnosing, for example, Alzheimer's disease, Parkonsinism, amyotrophic lateral sclerosis, and Huntington's disease.

Also included in the invention is a method for treating a subject having or suspected of having a disease, disorder, or condition characterized by reduced acetylation of a lysine at position 40 (K40) or αtubulin. The method includes administering to the subject a compound that increases or stimulates the amount or activity of an MEC-17-encoding RNA transcript or an MEC-17 polypeptide. Preferably the compound includes an agonist of an MEC-17 polypeptide, more preferably the compound includes an agonistic antibody.

In yet another aspect, the invention includes a method for treating a subject having or suspected of having a disease, disorder, or condition that depends upon or is characterized by acetylation of a lysine at position 40 (K40) of α-tubulin. The method includes administering to the subject a compound that reduces, inhibits or eliminates the amount of activity of an MEC-17-encoding RNA transcript or an MEC-17 polypeptide. Preferably the compound includes an antagonist of an MEC-17 polypeptide, more preferably the compound includes an antagonistic antibody. The disease is preferably an autoimmune disease wherein the administration of the compound is effective to treat or prevent a viral infection such as the human immunodeficiency virus (HIV).

Another aspect of the invention is a genetically modified eukaryotic cell having a mutation in a naturally occurring MEC-17 gene or a deletion of a naturally occurring MEC-17 gene, such that the MEC-17 polypeptide encoded by the MEC-17 gene is absent, present at lower levels, or has reduced activity compared to naturally occurring MEC-17 polypeptide. Preferably the cell exhibits a lower level or absence of αTAT activity compared to the αTAT activity levels in a wild-type cell. A eukaryotic cell of the invention may have an α-tubulin having undetectably or reduced acetylation of a lysine at position 40 (K40) compared to a-tubulin in a wild-type cell. Preferably the cell of the invention is a MEC-17 gene knockout Tetrahymena cell.

The invention further includes a method for producing non-acetylated microtubules. The method includes culturing a genetically modified eukaryotic cell having a mutation in a naturally occurring MEC-17 gene or a deletion of a naturally occurring MEC-17 gene, such that the MEC-17 polypeptide encoded by the MEC-17 gene is absent, present at lower levels, or has reduced activity compared to naturally occurring MEC-17 polypeptide under conditions and time sufficient to produce non-acetylated microtubules and isolating the non-acetylated microtubules.

Also included in the invention is a genetically modified cell that overexpresses MEC-17 polypeptide. The cell exhibits an increased level of αTAT activity compared to the αTAT activity levels in a wild-type cell. Preferably the wild-type cell does not express MEC-17 polypeptide. In one embodiment the cell is a Tetrahymena cell. In another embodiment the cell is a bacterial cell. In a preferred embodiment the cell is selected from a mammalian cell, a fish cell, a plant cell, an insect cell, and a protozoan cell.

The invention is also directed to a method for producing an MEC-17 polypeptide. The method includes culturing a genetically modified cell that overexpresses MEC-17 polypeptide under conditions and for a time sufficient to produce an MEC-17 polypeptide and isolating the MEC-17 polypeptide.

The invention is further directed to an in vitro method for assaying K40 α-tubulin acetylation activity. The method includes providing microtubules lacking detectable α-tubulin acetylation at position K40, contacting the microtubules with an MEC-17 enzyme fraction in the presence of acetyl coenzyme A, and detecting acetylation of the microtubules. Alternatively, the method is used for identifying an inhibitor of an MEC-17 polypeptide. The method includes providing microtubules lacking detectable α-tubulin acetylation at position K40, contacting the microtubules with an MEC-17 polypeptide in the presence of acetyl coenzyme A and a candidate compound, detecting acetylation of the microtubules, and comparing the level of K40 α-tubulin acetylation when microtubules are contacted with an MEC-17 polypeptide in the presence of acetyl coenzyme A and in the absence of the candidate compound. A reduced level of K40 α-tubulin acetylation in the presence of the candidate compound is indicative that the candidate compound is an inhibitor of an MEC-17 polypeptide. Preferably the microtubules are provided in the form of axonemes. The microtubules may be obtained from a eukaryotic cell that lacks αTAT activity. Preferably the cell lacking αTAT activity is a MEC-17 gene knockout Tetrahymena cell. Optionally, the microtubules have been enzymatically or chemically deacetylated in vitro. The method may further include detecting acetylation at position K40 of the α-tubulin using an anti-acetyl K antibody or detectably labeled acetyl-CoA.

Unless otherwise specified, “a,” “an,” “the,” and “at least one” are used interchangeably and mean one or more than one.

### BRIEF DESCRIPTION OF THE DRAWINGS

FIG. 1 shows multiple sequence alignment of the catalytic domain of MEC-17 homologs from diverse species. The following species abbreviations were used: Dm, Drosophila melanogaster (SEQ ID NOs:25 and 26); Tb, Trypanosome brucei (SEQ ID NO:27); Tc, Trypanosoma cruzi (SEQ ID NO:28); Xt, Xenopus tropicalis (SEQ ID NO:29); Xl, Xenopus laevis (SEQ ID NO:30); Hs, Homo sapiens (SEQ ID NO:31); Dr, Danio rerio (SEQ ID NO:32); Ci, Ciona intestinalis (SEQ ID NO:33); Tt, Tetrahymena thermophila (SEQ ID NO:34); Pt, Paramecium tetraurelia (SEQ ID NO:35); Ce, Caenorhabditis elegans Mec17 (SEQ ID NO:36); Gl, Giardia lamblia (SEQ ID NO:37); Ce, Caenorhabditis elegans W06B11.1 (SEQ ID NO:38).

FIG. 2 shows deletion of the MEC17 gene in Tetrahymena. Genomic DNA was purified from a wildtype, MEC17-KO and K40R ATU1 mutant strains and subjected to PCR with a pair of primers that amplify either the NRK18 sequence (positive control, left panel), or the MEC17 sequence with one primer located in the targeted region intended to be replaced by neo4 cassette (5′-ACGATAAGGTATAAGGAACAG-3′; SEQ ID NO:39) and another primer located outside of the targeted region of MEC17 (5′-AAGTTATCTATCTTATCCAGG-3′; SEQ ID NO:40) (middle panel), or a mixture of MEC-17 primers and NRK18 control primers that provide an internal control (right panel).

FIG. 3 shows MEC-17 is required for acetylation of K40 on α-tubulin in Tetrahymena. a-c, Wild-type (pre-fed with ink) and MEC17-KO (arrow) cells labeled with anti-acetyl-K40 mAb (6-11 B-1) and anti-tubulin antibodies. d-f, Wild-type (d), MEC17-KO (e) and K40R (f) Tetrahymena labeled with pan anti-acetyl-K antibodies. g-h, Western blots of cells (g) or cytoskeletons (h) probed with 6-11 B-1 mAb, pan anti-acetyl-K, anti-α-tubulin (12G10 mAb) and anti-histone hvl antibodies. Stars mark non-tubulin proteins. Arrows mark acetylated histones. i, Growth curves of Tetrahymena. j-1, Wild-type (left) and GFP-Mec17p overproducing (right) Tetrahymena cells analyzed for GFP (j) or 6-11 B-1 mAb immunofluorescence (k).

FIG. 4 shows silver-stained 2D SDS PAGE of ciliary proteins from either wildtype, MEC-17-KO and K40R strains of Tetrahymena (Maruta et al., 1986 J. Cell Biol. 103:571-579). Note a shift in the position of α-tubulin isoforms in the MEC-17-KO and K40R sample, consistent with loss of acetylation. The left side represents the more basic part of the gel.

FIG. 5 shows axonemes are more sensitive to oryzalin treatment in MEC-17-KO and K40R α-tubulin mutants of Tetrahymena. The graph on the left side illustrates the responses of wildtype, K40R and MEC-17-KO strains to oryzalin of varying concentration. Cells were suspended at 10⁵ cell/ml in SPP medium and incubated with the drug for 22 hr at 30° C. and the cell density was determined. On the right side there are images of cells that were either untreated (top row) or treated with 25 μM oryzalin for 12 hr, and subjected to immunofluorescence with an anti-α-tubulin (12G10) mAb.

FIG. 6 shows MEC-17 and WO6B11.1 are required for acetylation of K40 and contribute to touch sensation in C. elegans. a-f, Wild-type and mutant adult hermaphrodites were labeled using 6-11 B-1 mAb. Small and large arrows mark axons and cell bodies of TRNs, respectively. Scale bar 10 μm. g, Histogram quantifying touch responses. The error bars represent SEM. Asterisk marks significant difference when compared to K40 transgene mec-12(e1607) (p<0.0001). The following numbers of animals were tested: wild type, 69; mec-12(e1607), 49; mec-17(ok2109), 44; W06B11.1 (ok2415), 33; mec-17(ok2109) W06B11.1(ok2415), 140; K40 transgene mec-12(e1607), 84; Q40 transgene mec-12(e1607) 78; R40 transgene mec12(e1607) 75.

FIG. 7 shows depletion of mec17 mRNA in zebrafish by SP MOs. Total cDNA was made using randomly-primed mRNA isolated from either control (WT) or embryos injected with 5 bp mismatch MO (MIS), ATG-MO (ATG), or splice-site MO (SP), and amplified using either primers corresponding to the coding sequence of the β-actin gene (top panel) or primers that correspond to the sequence of mec17 between exon 1 and exon 5 (bottom panel). As controls, amplifications were performed on samples that lacked reverse transcriptase (−RT). Note that the β-actin gene primers gave a robust product of the expected size (100 bp) for all +RT samples (top). With the mec17 primers, a product of the predicted size (345 bp) is amplified in +RT reactions, except for embryos treated with MEC-17-SP MOs, which do not show a product, consistent with downregulation of mec17 mRNA, possibly by the nonsense-mediated mRNA decay pathway.

FIG. 8 shows MEC-17 is required for K40 acetylation in zebrafish and normal embryonic development. Control embryos (a, c, c′, c″, e) and embryos injected with MEC17-ATG morpholinos, 48 hr post fertilization (hpf). (b, d, d′, d″, f) were observed live (a,b) or subjected to immunofluorescence 48 hpf using either 6-11 B-1 mAb (c-d″) or Znp1 mAb (e,f), which recognizes synaptotagmin 1. c′ and d′ show higher magnifications of the areas boxed in c and d. c″ and d″ show higher magnifications of the areas of pronephrons that contain cilia (marked with arrows in c″ and d″). In e and f, arrows mark axons of peripheral neurons.

FIG. 9 shows MEC-17 controls the levels of microtubule acetylation in mammalian cells. a-h, Expression of Mm-MEC-17 in Ptk2 cells increases the levels of acetyl-K40 α-tubulin. Cells expressing either EGFP or EGFP and Mm-MEC17 were stained with 6-11 B-1 mAb and anti-α-tubulin antibodies. i, Depletion of Hs-MEC-17 in HeLa cells reduces the level of acetyl-K40 α-tubulin. Cells were transfected with either GFP or Hs-MEC17 siRNAs and after 50 hr, treated for 7 hr with either 300 nM trichostatin A (TSA, stock solution in DMSO) or DMSO alone. Cell lysates were analyzed by western blot probed with either 6-11 B-1 mAb (top, middle panels) or anti-α-tubulin mAb (bottom panel).

FIG. 10 shows MEC-17 has intrinsic, K40-specific α-TAT activity. a, Crude Tetrahymena and recombinant murine MEC-17 were used for in vitro acetylation reactions of MEC17-KO axonemes and analyzed by western using 6-11 B-1 and 12G10 mAb. b, In vitro acetylation assays were performed with GST-MmMEC-17 using axonemes isolated from either the MEC17-KO (K40) strain or a K40R α-tubulin mutant. The marker (M) is acetylated glutamate dehydrogenase (55.6 kD). c, Recombinant GST-MmMEC-17 directly acetylates purified tubulin from the MEC17-KO strain in vitro. d, Coomassie Blue-stained gel with either purified MEC17-KO tubulin (36 ng) or porcine brain tubulin (15 ng, 99% pure, Cytoskeleton Inc).

FIG. 11 shows a silver stained SDS-PAGE gel with proteins purified on a GST-bind column (Novagen) from bacteria expressing either GST or GST-Mm-MEC-17.

FIG. 12A shows MEC-17 activity tubulin acetylation activity on axonemes in vitro does not require a salt-labile axoneme-associated component. MEC 17-KO axonemes were used without any treatment or exposed to solutions containing NaCl (0-1 M) for 30 min at 4° C., washed and used for in vitro acetylation using recombinant GST-MmMEC-17. FIG. 12B shows MEC-17 prefers microtubules over unpolymerized tubulin as a substrate. In vitro acetylation assays were performed using either axonemes or tubulin purified from the MEC-17-KO strain. A comparison between lanes 3 and 4 (for reactions with purified tubulin) and lanes 7 and 8 and 9 and 10 (for reactions with axonemes) show that paclitaxel stimulates MEC-17 activity. Since paclitaxel promotes microtubule polymerization and stabilizes microtubules, these data suggest that MEC-17 prefers microtubules over dimeric tubulin as a substrate.

FIG. 13 shows MEC-17 acetylates by entering the microtubule lumen from the end. Axonemes purified from the MEC-17-KO strain of Tetrahymena were subjected to an in vitro acetylation assay with either with recombinant GST (A-C) or GST-Mm-MEC-17 (D-L) and subjected to double immunofluorescence with the anti-α-tubulin 12G10 mAb (darker gray) and pan acetyl-K antibodies (brighter gray). The different channel images were intentionally aligned with an offset to compare them side-by-side.

### DETAILED DESCRIPTION OF THE INVENTION

The present invention is directed to a group of enzymes which regulate the stability and dynamics of microtubules called α-tubulin acetyltransferase (αTAT). The invention is further directed to cells having modified levels of αTAT modification and methods of using said cells. For example, cells of the present invention may be used to screen for and identify compounds useful for inhibiting or enhancing αTAT activity. The present invention is also therefore directed to methods of using and identifying compounds that inhibit or enhance aTAT activity.

Tetrahymena is a free-living ciliate and a model eukaryote with a sequenced genome. Tetrahymena has been used in research that led to some key achievements, including the Nobel award winning discovery of self-splicing RNA, and identification of telomeres and telomerase, histone acetyltransferase, dynein, and siRNAs that guide DNA rearrangement (Turkewitz et al. (2002) Trends in Genetics 18, 35-40). The Tetrahymena system is well equipped for reverse genetics (by homologous DNA recombination), and biochemical studies (Collins et al. (2005) Curr Biol 15, R317-318).

Tetrahymena assembles a diverse set of microtubules including those forming the cell body networks, cilia, centriole-like basal bodies, and the mitotic spindle. Due to the importance of their diverse microtubules, ciliates are sensitive to perturbations in microtubule-dependent functions which result in characteristic (potentially screenable) phenotypes (Janke et al. (2005) Science 308, 1758-1762; Thazhath, et al. (2002) Nature Cell Biol. 4, 256-259, Fujiu et al. (2000) Cell Motil Cytoskeleton 46, 17-27).

Tetrahymena posttranslationally modifies its microtubules, using highly conserved post-translational modifications, including extensive acetylation on lysine 40 of α-tubulin (Akella et al., 2010 Nature 467:218-222; see Example II). Tetrahymena has been in use as a model to dissect the function of post-translational modifications. The use of Tetrahymena led to the first report a mutant phenotype caused by lack of specific sites of post-translational modifications on tubulins (Thazhath et al. (2002) Nature Cell Biol. 4, 256-259), and the discovery of the first post-translational modification forward enzyme (Janke et al. (2005) Science 308, 1758-1762).

Tetrahymena is attractive for high throughput manipulations due to its: 1) rapid growth (generation time of 3 hrs), 2) low cost of culture, 3) nearly transparent culture medium, 4) ability to grow on defined medium without animal products (lowers the cost of culture and reduced the strigency of required biosafety procedures, 5) lack of pathogenicity, 6) routine culture on 96-well plates, 7) growth to high density in microdrops (compatibility with 384-1536 well plates), 8) lack of autofluorescence (no cell wall or plastids), 9) rapid cell motility (promotes mixing of assay components), 10) sensitivity to established inhibitors (e.g. cycloheximide, paclitaxel) within the concentration range similar to animal cells, 11) methods for introduction of transgenes and protein tagging, 12) targeted mutation approaches that allow for exploration of loss-of-function phenotypes, 13) inducible-repressible promoters that allow for generation of gain-of-function phenotypes, 14) large cell size—highly amenable for HT cytological profiling at low microscopic magnification, 15) advanced cellular functions shared with animal cells including sophisticated microtubule-based organelles, regulated secretion, nuclear apoptosis, DNA rearrangements, phagocytosis, and chemotactic cell motility.

MEC-17 is a known protein that has been studied in one model, the worm C. elegans. Published studies showed that MEC-17 is required for maintenance of touch receptor cells (Chalfie and Au, 1989 Science 243:1027-1033; Zhang et al., 2002 Nature 418:331-335) and has sequence motifs that resemble the catalytic domain of the Gcn5 histone acetyltransferase (Steczkiewicz et al., 2006 Cell Cycle 5:2927-2930). However, these studies did not reveal the specific molecular function of MEC-17.

We now show that MEC-17 polypeptide, a highly conserved enzyme, acetylates α-tubulin, a subunit of microtubules, thereby identifying the substrate for MEC-17. The present invention thus establishes that MEC-17 polypeptide acts as an acetyltransferase for α-tubulin. We also show that MEC-17 is important in the vertebrate nervous system, leading to the expectation that it is also important in humans. We further show that MEC-17 protein is required for K40 α-tubulin acetylation in vivo in Tetrahymena, C. elegans and zebrafish. A recombinant murine MEC-17 protein was shown to modify microtubules on α-tubulin in vitro exclusively at K40 in the presence of acetyl-CoA. A depletion of MEC-17 protein in zebrafish led to loss of mobility and response of to touch, consistent with a neuromuscular defect.

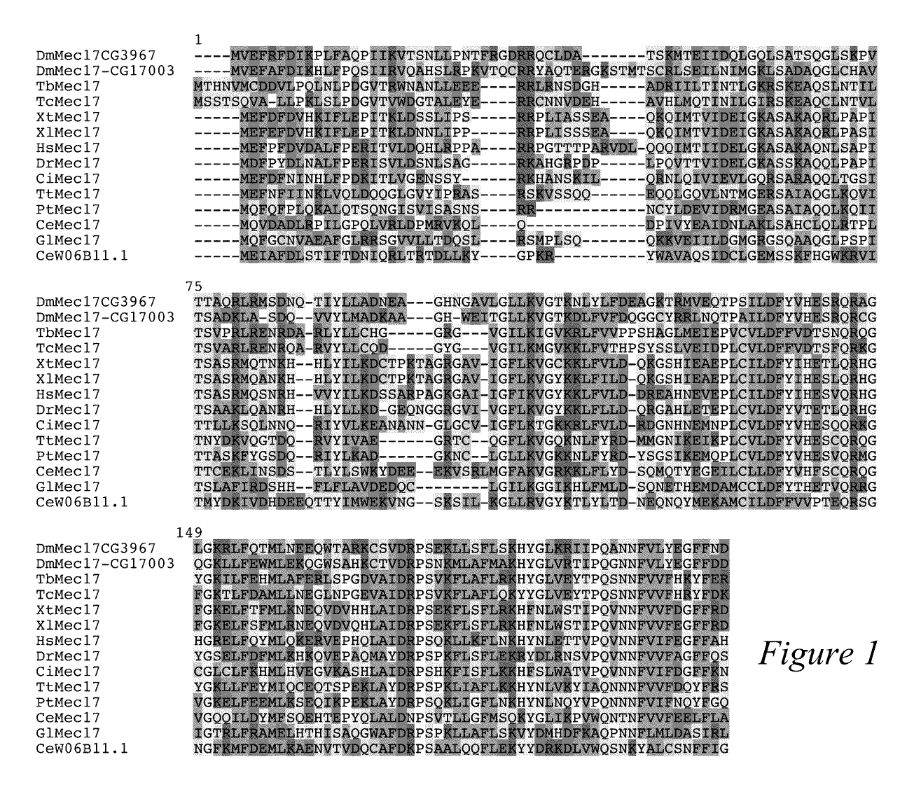

In this work we show that MEC-17 possesses acetyltransferase activity; more particularly, we show that MEC-17 functions as α-tubulin acetyltransferase (αTAT). Importantly, MEC-17 homologs are present in most eukaryotes with the exception of fungi and plants. See FIG. 1 for illustrative, non-limiting examples of MEC-17 homologs, such as a human MEC-17 (encoded by the c6orf134 locus), murine MEC-17 (Gene ID Q8K341) and zebrafish MEC-17 (zgc:65893 MEC-17). Thus, in this work we identify MEC-17 and MEC-17 homologs in eukaryotic organisms as α-tubulin acetyltransferases. It should be noted that the words “homolog” and “ortholog” are used interchangeably herein. It should be further noted that all descriptions of the invention herein that reference “MEC-17,” “MEC17,” “MEC-17,” etc., whether in the context of polypeptide, ribonucleic acid (RNA) or deoxyribonucleic acid (DNA) and uses thereof, or otherwise, are to be understood as including and being otherwise wholly applicable to MEC-17 orthologs and homologs in and from other eukaryotic systems, whether or not orthologs and homologs are specifically recited in the description of that aspect or embodiment of the invention. For example, in treatment or diagnostic methods involving a human, human ortholog(s) and/or homolog(s) of MEC-17 are intended to be included in, and are encompassed by, the use of the term “MEC-17.”

In animal cells, several acetyltransferases colocalize with acetylated microtubules and some regulate the level of acetyl-K40 α-tubulin, including N-acetyltransferase 1 (NAT) 1 (Ohkawa et al., 2008 Genes Cells 13:1171-1183), NAT10 (Shen et al., 2009 Exp. Cell Res. 315:1653-1667) and elongator protein 3 (ELP3) (Creppe et al., 2009 Cell 136:551-564; Solinger et al., 2010 PLoS Genet. 6:e1000820), but it is not known whether any of these enzymes have direct activity on α-tubulin. The present invention is also directed to additional enzymes that acetylate α-tubulin.

MEC-17 activity is especially important in the brain, more particularly in the cortex. Down regulation of MEC-17 was shown to lead to defects in neuronal morphology and delayed migration of cortical neurons during brain development in rats, which were partially correctable by a knockdown of HDCA6, an α-tubulin deacetylase (Li et al. J. Neurosci., 2012, 32:12673-12683). Experiments in C. elegans showed that touch receptor neurons with MEC-17 have fewer microtubules, with abnormal lattice organization and polarization (Topalidou et al., 2012, Curr. Biol. 22:1057-1065; Cueva et al., 2012, Curr Biol. 22:1066-1074). The abnormal organization of microtubules in touch receptor neurons could be caused by lack of K40 acetylation that may in turn have a role in increasing adhesion between the protofilaments of microtubules (Cueva et al., 2012, Curr Biol. 22:1066-1074).

Additionally, MEC-17 has bioactivity in addition to its enzymatic activity as an acetyltransferase. An enzymatically inactive form of MEC-17 can rescue touch sensation in MED-17 null mutants (Topalidou et al., 2012, Curr. Biol. 22:1057-1065). However, the inactive MEC-17 enzyme cannot rescue the defects in the organization of microtubules. Thus, this experiment shows that in the touch receptor neurons have some functionality even with severely compromised microtubules. The assays for MEC-17 activity described herein are based on acetylation of tubulin; however, they are expected to also identify inhibitors that bind to MEC-17 and inhibit both its enzymatic and non-enzymatic functions, for example by preventing MEC-17 from binding to tubulin or other substrates.

MEC-17 and MEC-17 homologs can be viewed at the National Center for Biotechnology Information website, available on the World Wide Web at www.ncbi.nlm.nih.gov/. MEC-17 and MEC-17 homologs can be identified by either an NCBI gi number or an NCBI accession number. Non-limiting examples of MEC-17 and MEC-17 homolog sequences include, for example, Caenorhabditis elegans mec-17 (gi|17540784|ref|NP_{—}501337.1|; SEQ ID NO:41); Caenorhabditis elegans W06B11.1 (gi|17570273|ref|NP_{—}508981.1|; SEQ ID NO:42); Ciona intestinalis (gi|198422961|ref|XP_{—}002123997.1|; SEQ ID NO:43); Drosophila melanogaster CG3967 (gi|21355363|ref|NP_{—}648310.1|; SEQ ID NO:424); Drosophila melanogaster CG17003 (gi|20129077|ref|NP_{—}608365.1| CG17003; SEQ ID NO:45); Danio rerio (gi|34784851|gb|AAH56749.1| Zgc:65893; SEQ ID NO:46); Giardia lamblia (gi|159112038|ref|XP_{—}001706249.1|; SEQ ID NO:47); Homo sapiens (gi|10435053|dbj|BAB14472.1|; SEQ ID NO:48); Micromonas sp. RCC299 (gi|255083454|ref|XP_{—}002504713.1|; SEQ ID NO:49); Physcomitrella patens subsp. patens (gi|168025715|ref|XP_{—}001765379.1|; SEQ ID NO:50); Paramecium tetraurelia (gi|145495487|ref|XP_{—}001433736.1; SEQ ID NO:51); Schistosoma japonicum (gi|56756064|gb|AAW26210.1| SJCHGC00609; SEQ ID NO:52); Trypanosoma brucei TREU927 (gi|72386619|ref|XP_{—}843734.1|; SEQ ID NO:53); Trypanosoma cruzi strain CL Brener (gi|71661627|ref|XP_{—}817832.1|; SEQ ID NO: 54); Tribolium castaneum (gi|189235028|ref|XP_{—}972158.2|; SEQ ID NO:55); Tetrahymena thermophila (gi|118354427|ref|XP_{—}001010476.1|; SEQ ID NO:56); Xenopus laevis (gi|148237085|ref|NP_{—}001089986.1|; SEQ ID NO:57); Xenopus (Silurana) tropicalis (gi|187937169|ref|NP_{—}001120781.1|; SEQ ID NO:58); Chlamydomonas reinhardtii (Cre07.g345150; SEQ ID NO:59). See Example IV.

Our invention provides a basis for assays for MEC-17 function both in vitro and in vivo, based on α-tubulin acetyltransferase activity. There is growing evidence that acetylation of K40 on α-tubulin is an important regulator of motor-driven trafficking inside cells, and in particular inside nerves. Our discovery is expected to lead to the development of compounds that modulate the nervous system, including approaches to treat neurodegenerative and mental disorders. Furthermore, a gene that encodes a mammalian ortholog of MEC-17 is located inside a major histocompatibility (MHC) region, in humans on chromosome 6 (locus C6orf134). This observation opens a possibility that MEC-17 and its activity in acetylation of microtubules is important during immune responses. There are reports in the literature that link acetylation of microtubules to immune synapse formation and viral infection (including HIV). Our invention thus leads to the expectation that drugs that affect MEC-17 protein could have use in treatment of immune disease, specifically autoimmune disorders and cancers. Furthermore, it is expected that a drug that blocks MEC-17 activity can also inhibit infection of immune cells by HIV.

The present invention thus includes both in vivo and in vitro methods for identifying compounds that enhance or inhibit an αTAT reaction. In vitro methods advantageously utilize cellular components such as axonemes, microtubules, or tubulins that are isolated from the genetically engineered Tetrahymena cells of the invention. The cellular components useful in the in vitro assays are components that are, in a wild-type Tetrahymena cell, acetylated. Also included in the present invention are the enhancers and inhibitors identified using the method of the invention.

A inhibitor or enhancer compound identified by the method of the present invention may be, without limitation, a protein, a peptide, a peptide fragment, an antibody, a small molecule, a nucleic acid, a chemical compound, or any other molecule capable of inhibiting or enhancing the αTAT reaction. A compound identified by the method of the present invention may be a natural or a synthetic compound. Also included in the present invention are compositions including an inhibitor or enhancer molecule identified by the method of the present invention. Such compositions typically include a pharmaceutically acceptable carrier. As used herein “pharmaceutically acceptable carrier” includes saline, solvents, dispersion media, coatings, antibacterial and antifungal agents, isotonic and absorption delaying agents, and the like, compatible with pharmaceutical administration.

Non-limiting examples of compounds of the invention that are useful for inhibiting a-tubulin acetylation include, for example, 3-carbethoxy-2-methylquinoline, 4-hydroxy-2-pentylquinoline-3-carboxylic acid (Mai et al., 2006 J. Med. Chem. 49:6897-6907), and the isothiazolone compound CCT077791 (Stimson et al., 2005 Mol Cancer Ther. 4:1521-1532).

MEC-17 is structurally related to histone acetyltransferases (HAT) and in particular to the MYST family of HATs. Currently, there are known inhibitors for p300 and Gcn5, two distinct classes of HATs (Vernarecci et al., 2009 Epigenetics 5:105-111), as well as known inhibitors for H3 acetylation (Mai et al., 2006 J. Med. Chem. 49:6897-6907). Thus, candidate inhibitor molecules include compounds already known to inhibit related molecules such as HATs.

In vivo assays may be performed using the cells of the present invention and may include biological assays or molecular assays. For example, a biological assay may involve phenotypic studies. Phenotypic studies typically used to examine microtubules include, but are not limited to, cell growth, cell motility, phagocytosis, and ciliary beat frequency. In one embodiment, an in vivo assay may be used as a primary screen and an in vitro assay may be used as a secondary screen, or vice versa.

In vitro assays may utilize isolated cellular components of the cells of the present invention. An “isolated” component, such as an axoneme, microtubule or tubulin, is one that has been removed from the cell. Preferably, the cellular component is purified, i.e., essentially free from any other cellular products or impurities. In vitro assays may include biological or molecular assays. For example, a molecular assay typically used to examine microtubules may include, without limitation, biochemical studies such as acetylation assays. For example, an in vitro assay with purified enzyme will determine whether the compound directly inhibits or enhances the αTAT activity. Further assays, both in vitro and/or in vivo, can be used to determine whether the enzyme is a broad or selective modulator and whether it can act on mammalian enzymes. These assays are commonly used in the field and would be known to a skilled artisan.

Assays for detecting acetylation are also well known in the art and are further described in the Examples, below.

Cellular components such as axonemes, microtubules or tubulin, which have been isolated from a Tetrahymena cell of the invention that exhibits reduced αTAT activity, are useful in in vitro assays for identifying inhibitors or enhancers of tubulin acetylation. For example, a substrate, such as an as axoneme, microtubule or tubulin, can be contacted with a candidate αTAT-inhibitor compound and a known αTAT. A change in the acetylation level of the substrate, dependent upon the presence or absence of the inhibitor compound, can be detected. The absence of additional acetylation, or a slower rate of acetylation in the presence of the candidate compound compared to the acetylation rate in the absence of the candidate compound, indicates that the compound functions as an αTAT inhibitor. Likewise, to identify an enhancer compound, an increase in acetylation amount or rate, compared to the acetylation rate in the absence of the candidate compound, is indicative of a compound that enhances or stimulates αTAT activity. Optionally, the inhibitory nature of an inhibitory compound can be confirmed or further characterized or studied using in vivo assays.

Alternatively, a primary in vivo assay can be used to identify candidate αTAT inhibitor compounds. In this embodiment, a Tetrahymena cell exhibiting reduced αTAT activity (and hence, hyperacetylation) as described herein can be contacted with a candidate aTAT-inhibitor compound, and a change in at least one of cell growth or motility can be detected. A decrease in cell growth or motility is indicative of αTAT inhibition. The method may further include detecting the amount of acetylation. A decrease in acetylation relative to an untreated cell is indicative of αTAT inhibition.

In one aspect, the invention provides a method for decreasing α-tubulin acetyltransferase activity in a cell. Preferably, the activity that is reduced or eliminated is K40 α-tubulin acetyltransferase activity. More generally, the invention contemplates reducing or eliminating the production or activity of one or more gene products of MEC-17 (or homologs/orthologs thereof), the gene that encodes the polypeptide MEC-17. A compound that inhibits, reduces, or eliminates the production or activity of a MEC-17 RNA transcript or a MEC-17 polypeptide (referred to herein as an inhibitor or an inhibitory compound) is introduced into the cell. An inhibitory compound, as discussed herein, can be any compound that reduces or eliminates the production or activity of MEC-17 (or homologs/orthologs thereof) including, without limitation, a polypeptide such as a protein (e.g., an antibody), peptide, glycoprotein and the like, including variants or derivatives thereof such as a peptidomimetic; a nucleic acid such as a DNA or RNA thereof such as antisense DNA or RNA, a ribozyme, an RNA or DNA aptamer, siRNA and the like, including variants or derivatives thereof such as a peptide nucleic acid (PNA); a carbohydrate such as a polysaccharide or oligosaccharide and the like, including variants or derivatives thereof; a lipid such as a fatty acid and the like, including variants or derivatives thereof; or a small organic molecules including but not limited to small molecule ligands, small cell-permeable molecules, and peptidomimetic compounds. Introduction of the inhibitor preferably results in a decrease in the acetylation of an α-tubulin, preferably a decrease in the acetylation of a lysine at position 40 (K40) of the α-tubulin. In some embodiments, decreasing the α-tubulin acetyltransferase activity in a cell is expected to have a therapeutic effect, as described herein.

Methods for reducing or eliminating the production or activity of one or more gene products of MEC-17 include, without limitation, those that act directly or indirectly on a MEC-17 gene; those that act directly or indirectly on the mRNA transcript(s) produced by this gene; those that affect the translation of the mRNA transcript(s) into a polypeptide; and those that inhibit the activity of the transcribed RNA or translated polypeptide.

Transcription of a gene can be impeded, or a gene can be silenced, by delivering to the cell an inhibitory compound such as antisense DNA or RNA molecule or a double stranded RNA molecule, such as small interfering RNA (siRNA), for example using RNA interference (RNAi); or by introducing into the cell DNAs operatively encoding such bioactive RNAs. The introduction of the inhibitory compound acts to directly or indirectly reduce or eliminate the production or activity of one or more gene products of MEC-17.

Another way the production of a polypeptide can be reduced is by interfering with the mRNA transcription product of the gene. For example, a compound such as a ribozyme (or a DNA vector operably encoding a ribozyme) can be delivered to the cell to cleave the target mRNA, thereby reducing or eliminating production of the polypeptide. Antisense nucleic acids and double stranded RNAs may also be used to interfere with translation.

A polypeptide product of a MEC-17 gene encoding an α-tubulin acetyl transferase e.g., MEC-17, can also be targeted. Antibodies or antibody-like molecules such as peptide aptamers can be introduced into the organism to reduce or abolish the activity of the translated polypeptide, as can protein or peptide antagonists or inhibitors such as linear or cyclic peptides, peptidomimetics, or small organic molecules. In general, this embodiment of the invention contemplates the use of any method that interferes with the production or activity of a MEC-17 gene, mRNA or encoded polypeptide.

In another aspect, the invention provides a method for increasing α-tubulin acetyl transferase acetyltransferase activity in a cell. Preferably, the activity that is increased or stimulated is K40 α-tubulin acetyltransferase activity. More generally, the invention contemplates increasing or stimulating the production or activity of one or more gene products of MEC-17 (or homologs/orthologs thereof), the gene that encodes the polypeptide MEC-17. A compound that increases or stimulates the production or activity of a MEC-17 RNA or a MEC-17 polypeptide (referred to herein as a stimulatory compound) is introduced into the cell. Introduction of the stimulatory compound preferably results in an increase in the acetylation of an α-tubulin, preferably an increase in the acetylation of a lysine at position 40 (K40) of the α-tubulin. In some instances, increasing MEC-17 acetyltransferase activity in a cell may have a therapeutic effect, as described herein.

Methods for increasing or stimulating the production or activity of one or more gene products of MEC-17 include, without limitation, those that act directly or indirectly on a MEC-17 gene; those that act directly or indirectly on the mRNA transcript(s) produced by this gene; those that affect the translation of the mRNA transcript(s) into a polypeptide; and those that increase the activity of the transcribed RNA or translated polypeptide.

Transcription of a gene can be enhanced by delivering to the cell a stimulatory compound such as an activator; or by introducing into the cell DNAs operatively encoding bioactive RNAs that can bind to DNA and stimulate transcription. The introduction of the stimulatory compound acts to directly or indirectly increase or stimulate the production or activity of one or more gene products of MEC-17.

The activity of a polypeptide product of a MEC-17 gene, e.g., MEC-17, can be increased by contacting the cell with agonistic (stimulating) antibodies or antibody-like molecules such as peptide aptamers, protein or peptide agonists or stimulants such as linear or cyclic peptides, peptidomimetics, or small organic molecules. In general, this embodiment of the invention contemplates the use of any method that enhances the production or activity of a MEC-17 gene, mRNA or encoded polypeptide.

The cell into which the inhibitor or stimulatory compound is introduced can be present, for example, in an organism, in a tissue or fluid that that has been removed from an organism, or in cell culture. The cell can be a prokaryotic or eukaryotic cell; it can be a plant or animal cell; it can be a bacterial cell, a protozoan cell, or an invertebrate or vertebrate cell. The cell is preferably a protozoan cell, a fish cell, an insect cell, a plant cell, such as an algae, or a mammalian cell; more preferably, it is a human cell.

It is expected that inhibition or stimulation of α-tubulin acetyltransferase activity (i.e., the activity of MEC-17 and/or its orthologs/homologs) will be useful to treat diseases, disorders and conditions of the nervous system and immune system including Alzheimer's disease, Huntington's disease, Parkinsonism, autoimmune disorders, and viral infections such as HIV infections and cancers that are characterized by or otherwise involve or are dependent upon acetylation levels of α-tubulin, preferably K40 of α-tubulin. Progressive motor neuron diseases such as amyotrophic lateral sclerosis (ALS) are also expected to be treatable by inhibition or stimulation of MEC-17. Recent evidence shows that ELP3, a regulator of K40 acetylation, is mutated in patients with ALS (Simpson et al., Hum. Mol. Gen. 2009 Feb. 1; 18(3):472-81). Additionally, detection of mutations, including insertions, deletions, or substitutions, in MEC-17 (or the human ortholog, encoded by the MEC-17/C6orf134 locus) is expected to be useful to diagnose said diseases, disorders and conditions. Compounds that affect, either directly or indirectly, the production or activity of MEC-17 polypeptide (e.g., inhibitory or stimulatory compounds as described herein) are useful to treat the aforementioned diseases, disorders and conditions, among others. Thus, in another aspect, the invention provides method for treating a subject having or suspected of having a disease, disorder or condition that can be treated by altering acetylation of an α-tubulin.

In one embodiment of the treatment method, the invention provides a method for treating a subject having or suspected of having a disease, disorder or condition that is characterized by or otherwise involves or is dependent upon reduced acetylation of α-tubulin polypeptide, preferably on K40 residue. In these subjects, reduced or low levels of acetylation of K40 on α-tubulin may be necessary for, associated with, or contribute to the pathology of the disease, disorder or condition. This embodiment of the treatment method involves administering to the subject, preferably a human subject, a compound that increases or stimulates the production or activity of MEC-17 RNA or a MEC-17 polypeptide (or homolog or ortholog thereof). An example of a stimulatory compound is an agonist of MEC-17 polypeptide, for example an agonistic anti-MEC-17 antibody.

In another embodiment of the treatment method, the invention provides a method for treating a subject having or suspected of having a disease, disorder or condition that depends upon or is characterized by or otherwise involves acetylation of K40 of α-tubulin polypeptide. Such diseases include some autoimmune disorders as well as some viral infections. In these subjects, the presence of acetylated K40 may be necessary for, associated with, or contribute to the pathology of the disease, disorder or condition. For example, acetylated microtubules may be required for communication between T-cells and antigen-presenting cells in the context of an autoimmune disease or for entry of HIV into the cell. This embodiment of the treatment method of the invention involves administering to the subject, preferably a human subject, a compound that reduces, inhibits or eliminates the production or activity of a MEC-17 RNA or a MEC-17 polypeptide (or homolog or ortholog thereof). An example of an inhibitor compound is an antagonist of MEC-17 polypeptide, for example an antagonistic anti-MEC-17 antibody. The inhibitor compound can be administered either therapeutically or prophylactically, for example prior to exposure to a pathogenic virus.

In another aspect, the invention provides a method for diagnosing a disease, disorder or condition of the nervous system or the immune system in a human subject. Exemplary diseases, disorders or conditions that can be detected include neurological, neurodegenerative, motor neuron and including Alzheimer's disease, Parkinsonism, amyotrophic lateral sclerosis, and Huntington's disease. The diagnostic method involves detecting a mutation in the human MEC-17/C6orf134 locus, or other genetic location encoding a homolog or ortholog of MEC-17. The mutation, which can take any form including an insertion, deletion, or substitution, affects the production or activity of MEC-17 polypeptide (or homolog or ortholog thereof).

In yet another aspect, the invention provides a genetically modified cell. The cell can be a eukaryotic cell or a prokaryotic cell. It can be a plant or animal cell, vertebrate or invertebrate. Preferably the genetically modified cell is a mammalian cell, a fish cell, an insect cell, a plant cell, such as an algae, a yeast cell, a protozoan cell or a bacterial cell. Unicellular eukaryotic cells, such as protozoan cells, are preferred. Even more preferably, the cell of the present invention is a ciliated cell. Examples of ciliated cells include, without limitation, Tetrahymena, Paramecium, Plasmodium, and Chlamydomonas. An example of a particularly preferred cell for use in the method of the invention is Tetrahymena. Also included in the present invention are cellular components, such as axonemes, microtubules or tublin, derived from the cells. The cells provided by the invention have been genetically engineered to either fail to express, to express reduced amounts of, or to overexpress, a MEC-17 polypeptide (or homolog or ortholog thereof).

In one embodiment, the genetically modified cell includes a mutation in or deletion of a naturally occurring MEC-17 gene, such that an enzymatically active MEC-17 polypeptide (or homolog or ortholog thereof) is not produced. An exemplary cell is a MEC-17 knockout Tetrahymena cell. Such cells assemble microtubules that fail to undergo acetylation by incorporating α-tubulin that is not acetylated at position K40. This cell is useful in a method for producing non-acetylated microtubules that is also provided by the invention. The cell is cultured under conditions and for a time sufficient to produce non-acetylated microtubules, and the non-acetylated microtubules are optionally isolated or purified. In Tetrahymena cells that lack the K40-specific αTAT, MEC-17, most if not all microtubules appear normal but cells are hyperresistant to paclitaxel and hypersensitive to oryzalin, indicating that MEC-17 has microtubule-stabilizing activity (Akella et al., 2010; Example II).

In another embodiment, a cell of the present invention has been engineered such that α-tubulin cannot be acetylated. For example, Tetrahymena mutants assemble diverse microtubules from α-tubulin and microtubules containing a K40R mutation cannot be acetylated (Gaertig et al., 1995 J. Cell Biol. 129:1301-1310)

Advantageously, due to its high growth rate and inexpensive methods of culture, Tetrahymena with deletions of MEC-17 is an excellent source of axonemes and other tubulin-containing structures for in vitro assay for tubulin acetylation at an industrial scale for the purpose of screening for compounds that either enhance or inhibit the reaction.

Antibodies that bind to axonemes, microtubules and tubulin isolated from cells of the invention, which have utility as diagnostic reagents and have potential therapeutic uses as well, are also included in the invention.

In another embodiment, the genetically modified cell overexpresses a MEC-17 polypeptide (or homolog or ortholog thereof). An enzyme is “overexpressed” in a genetically engineered cell when the active enzyme is expressed in the genetically engineered cell at a level higher than the level at which it is expressed in a comparable wild-type cell. In cells that do not endogenously express a particular enzyme, any level of expression of that enzyme in the cell is termed herein an “overexpression” of that enzyme for purposes of the present invention. For example, a bacterial cell such as an E. coli cell that is engineered to express a MEC-17 polypeptide is referred to herein as “overexpressing” MEC-17, since a bacterial cell does not naturally express MEC-17. Advantageously, MEC-17 can be expressed in a bacterial cell as a fusion protein, for example as a glutathione-sulfotransferase (GST) fusion protein. Another exemplary overexpressing cell is a Tetrahymena cell that overexpresses MEC-17 polypeptide. Overexpressing cells of the invention are useful in a method for producing MEC-17, an α-tubulin acetyltransferase enzyme. The cell is cultured under conditions and for a time sufficient to produce an α-tubulin acetyltransferase enzyme, and is α-tubulin acetyltransferase enzyme is optionally isolated and purified.

In another aspect, the invention provides an in vitro method for assaying MEC-17-mediated K40 α-tubulin acetylation activity. The method involves contacting non-acetylated microtubules with a sample, such as an enzyme fraction, suspected of or known to contain an MEC-17 polypeptide, in the presence of acetyl coenzyme A; and detecting acetylation of the microtubules, preferably at position K40 of the α-tubulin. Optionally, the non-acetylated microtubules are provided in the form of axonemes. The non-acetylated microtubules preferably lack detectable α-tubulin acetylation at position K40. They can be obtained from a eukaryotic cell that lacks MEC-17 activity, such as a MEC-17 knockout Tetrahymena cell. Alternatively, they can be obtained from a cell that contains α-tubulin that is naturally non-acetylated at position K40 or has low levels of acetylation at position K40 (such as PtK2 cells), or they can be enzymatically or chemically deacetylated in vitro. Detection of acetylation of the microtubules can be accomplished, for example, using an anti-acetyl-lysine (anti-acetyl-K) antibody or using detectably labeled, preferably radiolabeled, acetyl-CoA.

In yet another aspect, the invention provides a method for identifying an inhibitor of MEC-17 (or ortholog or homolog thereof). The method involves contacting microtubules lacking detectable α-tubulin acetylation at position K40 with an enzymatically active MEC-17 polypeptide in the presence of acetyl coenzyme A and a candidate compound; and comparing the level of K40 α-tubulin acetylation in the presence of the candidate compound with the level of K40 α-tubulin acetylation when microtubules are contacted with MEC-17 and acetyl coenzyme A in the absence of the candidate compound. A reduced level of K40 α-tubulin acetylation in the presence of the candidate compound is indicative that the candidate compound is an inhibitor of MEC-17.

Additionally, candidate compounds that inhibit or stimulate MEC-17 activity can be identified or designed in silico. Recently, two research groups have reported three-dimensional structures for α-tubulin acetyltransferase, paving the way for rational drug design and screening of molecules that inhibit or stimulate α-tubulin acetyltransferase activity. Modulators of acetyltransferase activity are important not only therapeutically, for both medical and veterinary applications, but also represent important research tools.

Taschner et al. reported an atomic resolution structure of human α-tubulin acetyltransferase catalytic domain bound to its co-substrate acetyl-CoA (Tascher et al., “Atomic resolution structure of human α-tubulin acetyltransferase bound to acetyl-CoA,” PNAS 2012; published ahead of print Oct. 15, 2012, doi:10.1073/pnas.1209343109), with supporting data available online at the address www.pnas.org/content/suppl/2012/10/11/1209343109.DC Supplemental. The authors report that the enzyme displays a unique active site and putative α-tubulin binding site. Mapping of the residues important for acetyl-CoA binding, substrate binding, and catalysis revealed a basic patch implicated in substrate binding and a conserved glutamine residue required for catalyst.

Friedmann et al. reported the X-ray crystal structure of human αTAT1/actetyl-CoA complex. Using the crystal structure in conjunction with structure-based mutagenesis, enzymatic analysis, and functional studies in cells, they elucidated the catalytic mechanism and mode of tubulin-specific acetylation. They identified conserved aspartic acid and cysteine residues that play important analytic roles through a ternary complex mechanism. These advances will facilitate the development of small molecule modulators of microtubule acetylation for therapy. Friedmann et al. remark on the ability of cancer cells to develop resistance to therapies through multidrug resistance cellular efflux, and notes that the structure of αTAT1 provides a unique enzyme scaffold for designing small molecule modulators of microtubule acetylation for therapeutic use. Their successful identification of αTAT1 mutations that modulate αTAT1 activity shows that it is possible to identify small molecule compounds that either increase or decrease αTAT1 activity to stabilize microtubules or to promote their depolymerization, respectively, for therapeutic purposes.

Thus, with three dimensional molecular structures available, computational techniques can be used to screen, identify, select and design chemical entities capable of associating with MEC-17 or structurally homologous molecules with αTAT activity. Knowledge of atomic coordinates permits the design and/or identification of synthetic compounds and/or other molecules capable of binding to the active site or in allosteric positions. Computational techniques can be used to identify or design compounds that are potential modulators of αTAT activity including inhibitors, enhancers, agonists, antagonists and the like. Potential modulators may bind to or interfere with all or a portion of the active site of MEC-17 or homologous molecules, and can be competitive, non-competitive, or uncompetitive inhibitors. Once identified and screened for biological activity, these modulators may be used therapeutically or prophylactically to inhibit or stimulate αTAT activity.

Data stored in a machine-readable storage medium that displays a graphical three-dimensional representation of the structure of MEC-17 or a structurally homologous αTAT molecule or domain, or portions thereof may thus be advantageously used for drug discovery. In rational drug design, the structure coordinates of a candidate drug are used to generate a three-dimensional image that can be computationally fit to the three-dimensional image of MEC-17 or a structurally homologous αTAT molecule or domain. The three-dimensional molecular structure encoded by the data in the data storage medium can then be computationally evaluated for its ability to associate with chemical entities. When the molecular structures encoded by the data are displayed in a graphical three-dimensional representation on a computer screen, the protein structure can also be visually inspected for potential association with chemical entities.

Candidate drugs screened in silico can be known compounds, or they can be newly designed compounds. If newly designed, they can be designed in a step-wise fashion, one fragment at a time, or may be designed as a whole or “de novo.”

If computer modeling indicates a strong interaction, the molecule may be synthesized and tested for its ability to bind to MEC-17 or interfere with αTAT activity. In general, binding assays to determine if a compound actually binds to a protein can also be performed, and are often well known in the art. Binding assays may employ kinetic or thermodynamic methodology using a wide variety of techniques including, but not limited to, microcalorimetry, isothermal denaturation, circular dichroism, capillary zone electrophoresis, nuclear magnetic resonance spectroscopy, fluorescence spectroscopy, and combinations thereof.

One skilled in the art may use one or more of several methods to screen compounds for their ability to associate with MEC-17 and affect αTAT activity. This process may begin by computer-assisted visual inspection during with the compound can be positioned in a variety of orientations, or docked, within the substrate binding surface or binding site. Docking may be accomplished using software such as QUANTA and SYBYL, followed by energy minimization and molecular dynamics with standard molecular mechanics forcefields, such as CHARMM and AMBER.

Candidate compounds may be designed “de novo” using either an empty binding site or optionally including some portion(s) of a known modulator(s). There are many de novo ligand design methods including, without limitation, LUDI (H.-J. Bohm, J. Comp. Aid. Molec. Design., 6:61-78 (1992); available from Molecular Simulations Inc., San Diego, Calif.); LEGEND (Y. Nishibata et al., Tetrahedron, 47:8985 (1991); available from Molecular Simulations Inc., San Diego, Calif.); LeapFrog (available from Tripos Associates, St. Louis, Mo.); and SPROUT (V. Gillet et al., J. Comput. Aided Mol. Design, 7:127-53 (1993); available from the University of Leeds, UK).

Once a compound has been designed or selected by the above methods, the efficiency with which that entity may bind to or interfere with a MEC-17/aTAT substrate binding surface or binding site may be tested and optimized by computational evaluation.

The present invention is illustrated by the following examples. It is to be understood that the particular examples, materials, amounts, and procedures are to be interpreted broadly in accordance with the scope and spirit of the invention as set forth herein.

### EXAMPLES

The present invention is illustrated by the following examples. It is to be understood that the particular examples, materials, amounts, and procedures are to be interpreted broadly in accordance with the scope and spirit of the invention as set forth herein.

### Example I

MEC17 protein was originally identified by Chalfie laboratory as a gene whose product is needed for maintenance of touch receptors in C. elegans (Way and Chalfie, 1989 Genes Dev 3:1823-1833; Zhang et al., 2002 Nature 418:331-335). Larve homozygous for a loss of function mutation in MEC 17 gene are touch sensitive but adult forms are not (Way and Chalfie, 1989 Genes Dev 3:1823-1833). Thus, the function of MEC17 is not needed for development of the touch receptor neurons but it is needed for maintenance of their functionality. MEC 17 is expressed primarily in the touch receptor neurons in C. elegans and it is one of the 50 or so genes whose expression is activated by MEC-3, a transcription factor that controls the touch receptor neuronal differentiation (Zhang et al., 2002 Nature 418:331-335).

Additional studies showed that the touch receptor neurons have unique microtubules that are made of 15 protofilaments (usually microtubules have 13 protofilaments) (Fukushige et al., 1999 J Cell Sci 112:395-403; Savage et al., 1989 Genes Dev 3:870-881). Also, touch receptors are one of the few if not the only place in the worm where α-tubulin (encoded by the MEC-12 gene) undergoes acetylation at K40 (Fukushige et al., 1999 J Cell Sci 112:395-403). This modification was originally discovered in flagella of the green alga Chlamydomonas reinhardtii (L'Hernault and Rosenbaum, 1983 J Cell Biol 97:258-263; L'Hernault and Rosenbaum, 1985 Biochem 24:473-478) and was later found to be highly conserved (LeDizet and Piperno, 1991 Meth Enzymol 196:264-274). Worms have several isotypes of α- and β-tubulin. MEC-12 encodes the only α-tubulin that has K40 and MEC12 protein is restricted to touch receptor neurons and a few other types of neurons (Fukushige et al., 1999 J Cell Sci 112:395-403). Mutations in MEC-12 α-tubulin lead to loss of 15 protofilament microtubules and loss of touch sensitivity (Driscoll and Tavernarakis, 1997 Gravit Space Biol Bull 10:33-42; Fukushige et al., 1999 J Cell Sci 112:395-403). Thus, the early literature was suggestive of a function of K40 acetylation in the sensory neurons based on the fact that the only α-tubulin that can be acetylated at K40 is expressed in the touch receptors in the worm. Fukushige and colleagues have attempted to address the function of K40 acetylation genetically. They found that a MEC-12 loss-of-function mutant can be rescued (to recover touch sensation) by introduction of a mutated MEC-12 α-tubulin gene having a K40Q substitution (Fukushige et al., 1999 J Cell Sci 112:395-403). However, a K40Q substitution is lysine acetylation-mimicking mutation (glutamine has a shape that is similar to acetylated lysine). Thus, this experiment indicates that removal of acetylation from α-tubulin (deacetylation) is not important in the worm but it does not address the significance of deposition of acetylation at K40. A conceptually similar experiment has recently been done in the mouse. HDAC6 is a major deacetylase enzyme that removes acetylation from acetylated K40 (Hubbert et al., 2002 Nature 417:455-458). Mice entirely lacking the HDAC6 gene are normal except for minor defects in the bone development and weaker immune responses. Moreover the levels of K40 acetylation greatly increase in the sperm and brain of the HDAC6-null mice which is expected because acetylation is not being balanced by deacetylation (Zhang et al., 2008 Mol Cell Biol 28:1688-1701). These data argue that removal of acetylation is not essential in worms and mammals but they do not address the importance of the forward step in the reaction—the attachment of the acetate group to the epsilon amino group of K40.

There are also surprising data on the role of K40 α-tubulin acetylation in protists. A K40R substitution (that makes the site non-acetylatable but preserves the basic charge similar to original lysine) in Tetrahymena failed to produce a major mutant phenotype (Gaertig et al., 1995 J Cell Biol 129:1301-1310). A similar conclusion was made based on lack of detectable consequences of ectopic expression of K40R and K40A α-tubulin in Chlamydomonas (Kozminski et al., 1993 Cell Motil Cytoskel 25:158-170). Based on these studies, one can conclude that the function of K40 acetylation is subtle in protists. However, our own unpublished data indicate that K40 acetylation regulates the dynamics of microtubules in Tetrahymena and possibly in other organisms (see below).

Our goal was to identify the acetyltransferase enzyme for K40 on α-tubulin. Steczkiewicz and other have noticed that MEC 17 homologs of diverse organisms have a conserved DUF738 domain (domain of unknown function 738) that shows weak homology to Gcn5-related acetyltransferases (Steczkiewicz et al., 2006 Cell Cycle 5:2927-2930). Gcn5 acetylates core histone proteins and regulates activation of transcription (Brownell et al., 1996 Cell 84:843-851). Based on the homology, Steczkiewicz and colleagues have proposed that MEC17 is another acetyltransferase that modifies histone proteins perhaps in cooperation with MEC-3 protein, to activate transcription of touch receptor genes (Steczkiewicz et al., 2006 Cell Cycle 5:2927-2930).

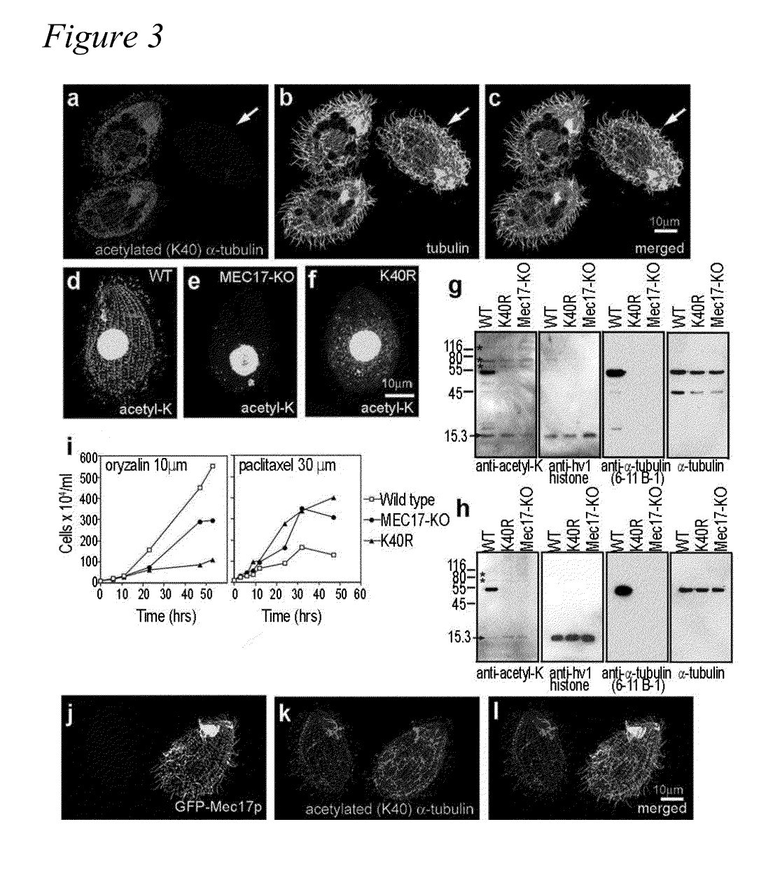

However, we have noticed, based on the literature, that there is a correlation between the expression of MEC 17 and the presence of acetylation of K40 α-tubulin in the same cell types in C. elegans. We hypothesized that MEC17 is an acetyltransferase for K40 on α-tubulin. To test this hypothesis, we deleted the single MEC17 gene in Tetrahymena thermophila. This led to a complete loss of α-tubulin K40 acetylation from cilia and cortical microtubules, based on the use of 6-11 B-1 antibody that is specific to acetyl-K40 (see Example II and FIG. 3a). Interestingly, MEC17 knockout cells have a weak signal of acetyl-K40 on macronuclear and micronuclear microtubules during cell division (results not shown), suggesting that another acetyltransferase capable of modifying K40 exists, and that in Tetrahymena this enzyme acts inside nuclei. Importantly, plants do not have a MEC 17 gene but do have acetylated K40 on α-tubulin. Thus, we suspect that plants and other organisms including Tetrahymena have another tubulin acetyltransferase that is related structurally to MEC 17 protein.

An antibody specific to acetylated lysine (acetyl-K) showed that MEC 17 knockout cells loose signal of lysine acetylation in cilia and cell cortex (that are enriched in microtubules), but not in nuclei, indicating that MEC 17 is required for acetylation of a microtubule-associated protein (see Example II and FIG. 3d-f).

A western blot showed that deletion of MEC 17 gene leads to complete loss of detectable K40 acetylation on α-tubulin in total cells and in the cytoskeletal fraction (detergent resistant fraction that contains most microtubules) of Tetrahymena. Antibodies that are specific to acetylated lysine (anti-acetyl-K), revealed several protein bands in wildtype Tetrahymena, but only the band that corresponds in size to α-tubulin was lost in the MEC17 knockout. These data taken together with those obtained using the 6-11 B-1 antibody that specifically recognized K40 on α-tubulin indicate that in Tetrahymena, MEC17 protein is specifically required for K40 acetylation on α-tubulin and not for acetylation of other proteins.

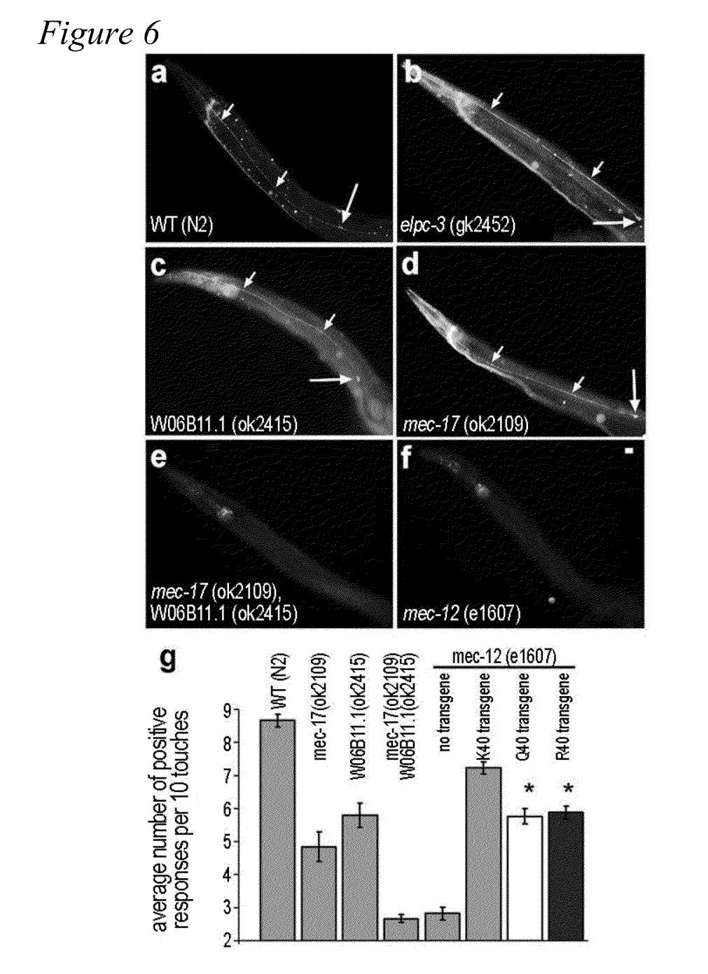

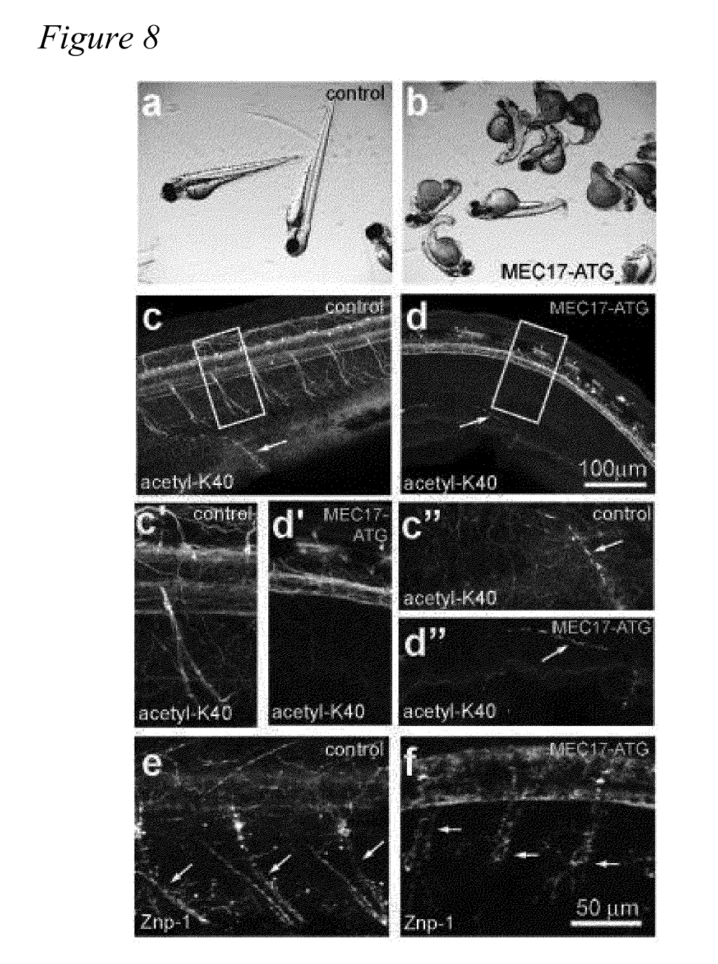

Next, we depleted the levels of MEC17 mRNA by injection of specific morpholinos into developing zebrafish embryos. Injection of MEC17 MOs slowed the rate of embryonic development caused swelling of the brain, and reduced the size of eyes in developing embryos at 48 hpf (see Example II and FIG. 6). The morphant embryos were insensitive to touch when probed with a needle (results not shown). At a later time, the embryos injected with MEC 17 morpholinos were severely deformed and died a few days later (see Example II and FIG. 8a-b). Axons of peripheral neurons in the trunk and tail had greatly reduced levels of K40 acetylation based on 6-11 B-1 antibody labeling (see Example II and FIG. 3g-h) but these neurons developed normal size axonal projections based on the staining with antibodies that recognize Znp-1 protein (see Example II and FIG. 3g-h). These data suggest that most if not all neurons in zebrafish deficient in MEC17 develop but lack acetylation of K40 on a-tubulin and that this modification is required for touch sensation and brain development.

These data so far indicate that MEC17 protein either acetylates α-tubulin on K40 directly or activates (acetylates?) another protein that in turn activates the unknown acetyltransferase for K40 on α-tubulin. To address the above question, we tested whether MEC17 has an intrinsic activity as an acetyl-transferase for K40 on α-tubulin. First we established an in vitro K40 α-tubulin acetylation activity assay composed of:

1) purified axonemes of the MEC17 Tetrahymena knockout strain (these axonemes entirely lack detectable K40 acetylation);

2) MEC 17 enzyme fraction; and

3) acetyl-coenzyme-A as a donor of activated acetate.

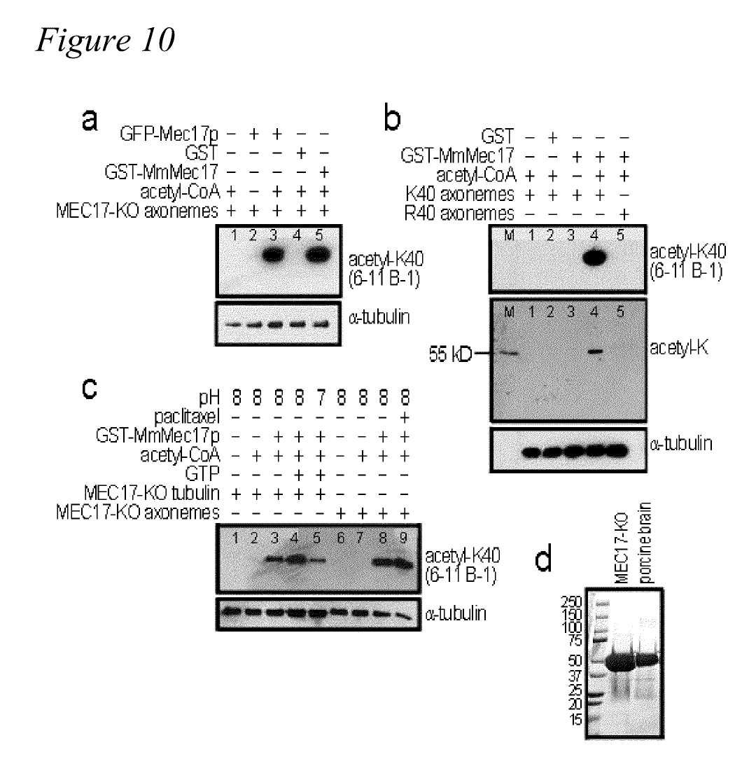

We showed that a soluble fraction of Tetrahymena cells overexpressing Tetrahymena own MEC17 protein contains increased K40 α-tubulin acetylation activity in vitro that was dependent on acetyl-coenzymeA (see Example II and FIG. 10b). Next, we expressed a murine MEC17 protein as a GST fusion in E. coli. The protein was purified as a GST fusion and GST protein alone was also purified as a negative control. GST-murine MEC17 and not GST alone strongly modified MEC 17 Tetrahymena knockout axonemes in vitro (see Example II and FIG. 10b). Thus, we establish that MEC17 protein has an intrinsic activity as a K40 a-tubulin acetyltransferase. Furthermore, we establish an in vitro assay for MEC17-mediated tubulin acetyltransferase activity.

### Implications of the Discovery

There is increasing evidence that K40 acetylation of α-tubulin of microtubules is important in regulation of organelle trafficking inside cells, and specifically inside nerves. Reed et al showed that kinesin-1, a microtubule-dependent motor protein that is targeted specifically to the axons (and not to dendrites) of neurons, binds to microtubules that have K40 acetylation with higher affinity as compared to non-acetylated microtubules (Reed et al., 2006 Curr Biol 16:2166-2172). Moreover, inhibitors of HDAC6 (a tubulin deacetylase) increased K40 acetylation on microtubules in vivo and stimulated the transport of kinesin-1 cargo, JIP1, into neurite extensions in neural cells (Reed et al., 2006 Curr Biol 16:2166-2172). K40 acetylation may exist at higher levels on axonal microtubules as opposed to dendritic or cell body microtubules, in particular in the most proximal part of the axon called the initial segment (Shea, 1999 Brain Res Bull 48:255-261). Thus, acetylation could be a determinant that directs motor proteins into the axon extension and thus promotes synaptic transport. In agreement with this model, a motor protein, kinesin-1 added to neurons devoided of the plasma membrane, bound selectively to the initial segment microtubules of the axon (Nakata and Hirokawa, 2003 J Cell Biol 162:1045-1055).

Tau is a MAP protein that is highly enriched in axons of nerves (Binder et al., 1985 J Cell Biol 101:1371-1378). Hyperphosphorylated Tau forms aggregates that accumulate in the brains of Alzheimer disease patients (reviewed in Gong and Iqbal, 2008 Current Med Chem 15:2321-2328). Mutations in Tau also contribute to dementia in Parkinsonism and other neurodegenerative disorders (reviewed in Sahara et al., 2008 Current Alzheimer Res 5:591-598). Importantly, there is a functional link between K40 acetylation on α-tubulin and Tau. K40 acetylation is the only post-translational modification of tubulin that occurs inside the microtubule lumen (Downing and Nogales, 1998 Curr Opin Cell Biol 10:16-22; Nogales et al., 1999 Cell 96:79-88). Tau binds to the surface microtubules (Al-Bassam et al., 2002 J Cell Biol 157:1187-1196). However, a microtubule stabilizing drug paclitaxel binds inside the lumen (Downing and Nogales, 1998 Curr Opin Cell Biol 10:16-22) and competes with Tau binding, and additional data suggest that Tau can also interact with the luminal side of microtubules (Kar et al., 2003 EMBO J 22:70-77). Thus, K40 acetylation could regulate Tau binding inside the microtubule lumen. Moreover, there is a piece of genetic evidence in support of this view. A Tau-like protein of C. elegans, PTL-1, is expressed only in small number of cell types, notably including touch receptor neurons. A mutation of PTL-1 resulted in reduced touch sensitivity and interacted genetically with MEC-12, the only K40 α-tubulin of C. elegans (Gordon et al., 2008 Devel Genes Evol 218:541-551). Thus, we can hypothesize that one of the functions of K40 α-tubulin acetylation is to regulate interactions of microtubules with proteins that bind to the lumen of microtubules, including Tau and other protein complexes that occupy the lumen (Garvalov et al., 2006 J Cell Biol 174:759-765; Nicastro et al., 2006 Science 313:944-948). One can therefore predict that mutations in MEC17 could contribute to pathology of Alzheimer's disease, Parkinsonism and other neurological disorders

K40 acetylation could also play a role in the Huntington disease (HD). The levels of K40 acetylation are reduced in the HD brains. Moreover, inhibitors of HDAC6, an α-tubulin K40 deacetylase, increase the levels of K40 acetylation and compensate for some transport deficiences that are found in neurons affected by HD (Dompierre et al., 2007 J Neurosci 27:3571-3583).

In humans, there is only a single gene that encodes MEC17 ortholog, encoded by the C6orf134 gene (unpublished data based on Pubmed databases). This gene is located on chromosome 6 inside the region occupied by genes that form the Major Histocompatibility Complex, MHC (Horton et al., 2008 Immunogenetics 60:1-18). It is well established that most if not all genes located in MHC are involved with the immune system. The location of MEC17/C6orf134 inside MHC is highly conserved, as MEC17 is also located in proximity of established MHC genes in non-mammalian vertebrates including zebrafish (Sambrook et al., 2005 BMC Genomics 6:152). The presence of MEC17 gene inside MHC suggests that its expression needs to be co-regulated with other MHC genes in the context of immune responses. Moreover, there is evidence that K40 acetylation on α-tubulin is involved in the immune responses. Acetylated microtubules accumulate in T cells when they establish cell-cell contacts with antigen-presenting cells (APC) (Serrador et al., 2004 Immunity 20:417-428). This process is known as establishment of the “immunological synapse” and involves transfer of information between APC and T cell. Overexpression of HDAC6 deacetylase led to reduction in the levels of K40 acetylation on microtubules near the immunological synapse and at the same time inhibited its function (Serrador et al., 2004 Immunity 20:417-428). Mice lacking HDAC6 gene have weakened immune responses (Zhang et al., 2008 Mol Cell Biol 28:1688-1701). All these data suggest that acetylation of K40 on α-tubulin in immune cells is important for their function. Thus, an inhibitor of MEC 17 protein could affect that levels of K40 acetylation and therefore could modulate immune responses. However, it remains to be determined whether MEC17 protein acetylates microtubules in immune cells.

Moreover, K40 acetylation could be an important regulator of viral infection, specifically in the context of HIV. There is an increase in K40 α-tubulin acetylation on microtubules in association with entry of HIV-1 into lymphocytes. Overexpression of HDAC6 decreases the levels of K40 acetylation on microtubules and at the same time inhibits HIV-1 viral infection (Malinowsky et al., 2008 Virology 376:69-78; Valenzuela-Fernandez et al., 2005 Mol Biol Cell 16:5445-5454). Increased acetylation of microtubules is also associated with infection by human herpex virus 8 (HHV-8) and herpes simplex virus-1 (HSV-1) (reviewed in Serrador et al., 2004 Immunity 20:417-428). One can therefore make a prediction that blocking α-tubulin acetylation via blocking MEC 17 activity in cells, should inhibit viral infections. Thus, an inhibitor or MEC17 could be useful in preventing viral re-infections and could help to drive the viral load down in infected patients.

It is important to add that there are established fluorescence based assays for protein acetyltransferase activity that are based on measuring accumulation of an acetyl-coenzyme A product (Wu and Zheng, 2008 Analytical Biochem 380:106-110). These assays have already been used in screens for inhibitors of histone acetyltransferases (HATs). Thus, the assay that we have developed for MEC 17 α-tubulin K40 acetylation in vitro can now be applied for high-throughput screens of inhibitors of MEC17 by a simple modification.

### Conclusion

The invention establishes that MEC 17 acts as an α-tubulin acetyltransferase. The invention provides an in vitro assay for detecting activity of MEC17 α-tubulin acetyltransferase activity and opens way for large scale screens for inhibitors of MEC17 activity. The invention calls attention to MEC 17 (C6orf134) locus in humans as a potential site of mutations that could play a role in neurodegenerative diseases and other disorders of the nervous system as well as in immune diseases. The invention predicts that a chemical stimulator of MEC 17 activity could be useful in treatment of the Huntington disease. On the other site, an inhibitor of MEC 17 protein activity could be useful in reducing the immune responses and therefore has a potential in treatment of autoimmune diseases. Finally, an inhibitor of MEC17 protein could prevents infection of cells by viruses including HIV.

### Example II

### MEC-17 is an α-Tubulin Acetyltransferase

In most eukaryotic cells, subsets of microtubules are adapted for specific functions by post-translational modifications (PTMs) of tubulin subunits. Acetylation of the ε-amino group of K40 on α-tubulin is a conserved PTM on the luminal side of microtubules (Nogales et al., 1999 Cell 96:79-88) that was discovered in the flagella of Chlamydomonas reinhardtii (L'Hernault and Rosenbaum, 1983 J. Cell Biol. 97:258-263; LeDizet and Piperno, 1987 Proc. Natl. Acad. Sci. USA 84:5720-5724). Studies on the significance of microtubule acetylation have been limited by the undefined status of the α-tubulin acetyltransferase. Here, we show that MEC-17, a protein related to the Gcn5 histone acetyltransferases (Steczkiewicz et al., 2006 Cell Cycle 5:2927-2930) and required for the function of touch receptor neurons in C. elegans (Chalfie and Au, 1989 Science 243:1027-1033; Zhang et al., 2002 Nature 418:331-335), acts as a K40-specific acetyltransferase for α-tubulin (see, e.g., Akella et al., “MEC-17 is an alpha-tubulin Acetyltransferase,” 2010 Nature 467:218-222). In vitro, MEC-17 exclusively acetylates K40 of α-tubulin. Disruption of the Tetrahymena MEC-17 gene phenocopies the K40R α-tubulin mutation and makes microtubules more labile. Depletion of MEC-17 in zebrafish produces phenotypes consistent with neuromuscular defects. In C. elegans, MEC-17 and its paralog W06B11.1 are redundantly required for acetylation of MEC-12 α-tubulin, and contribute to the function of touch receptor neurons partly via MEC-12 acetylation and partly via another function, possibly by acetylating another protein. In summary, we identify MEC-17 as an enzyme that acetylates the K40 residue of α-tubulin, the only PTM known to occur on the luminal surface of microtubules.

### Results and Discussion

Acetyl-K40 marks are enriched on a subset of microtubules that turnover slowly (reviewed in Verhey and Gaertig, 2007 Cell Cycle 6:2152-2160). The K40 residue of a-tubulin is not required for survival in protists, such as Tetrahymena (Gaertig et al., 1995 J. Cell Biol. 129:1301-1310), or Chlamydomonas (Kozminski et al., 1993 Cell Motil. Cytoskel. 25:158-170) but appears to be important in vertebrates. In neurons, axonal microtubules have higher levels of K40 acetylation than dendritic microtubules (Witte et al., 2008 J Cell Biol 180:619-632). Neurons that overexpress a K40A mutant α-tubulin show altered motor-based trafficking and cell differentiation (Dompierre et al., 2007 J Neurosci 27; Creppe et al., 2009 Cell 136:551-564). Kinesin-1, a motor that is preferentially targeted to the axon (Nakata and Hirokawa, 2003 J Cell Biol 162:1045-1055), has higher affinity for acetylated as compared to non-acetylated microtubules (Dompierre et al., 2007 J Neurosci 27:3571-3583; Reed et al., 2006 Curr Biol 16:2166-2172; Konishi and Setou, 2009 Nature Neuroscience 12:559-567). Two histone deacetylase-related enzymes, HDAC6 and SIRT2, deacetylate α-tubulin (Hubbert et al., 2002 Nature 417:455-458; North et al., 2003 Mol Cell 11:437-444). The α-tubulin acetyltransferase (αTAT) has been partially purified (Maruta et al., 1986 J. Cell Biol. 103:571-579) but the identity of the catalytic subunit remains unknown. Recently Steczkiewicz and colleagues reported that the conserved protein domain DUF738 has weak amino acid sequence homology to the catalytic domain of the Gcn5 histone acetyltransferases (Steczkiewicz et al., 2006 Cell Cycle 5:2927-2930). Among the DUF738 proteins is MEC-17, whose activity is required for the maintenance of touch receptor neurons (TRNs) in C. elegans (Chalfie and Au, 1989 Science 243:1027-1033; Zhang et al., 2002 Nature 418:331-335). Intriguingly, in C. elegans, acetylated α-tubulin (MEC-12) is enriched in the TRNs (Fukushige et al., 1999 J. Cell Sci. 112:395-403). These observations opened the possibility that MEC-17 is involved in K40 acetylation on α-tubulin.

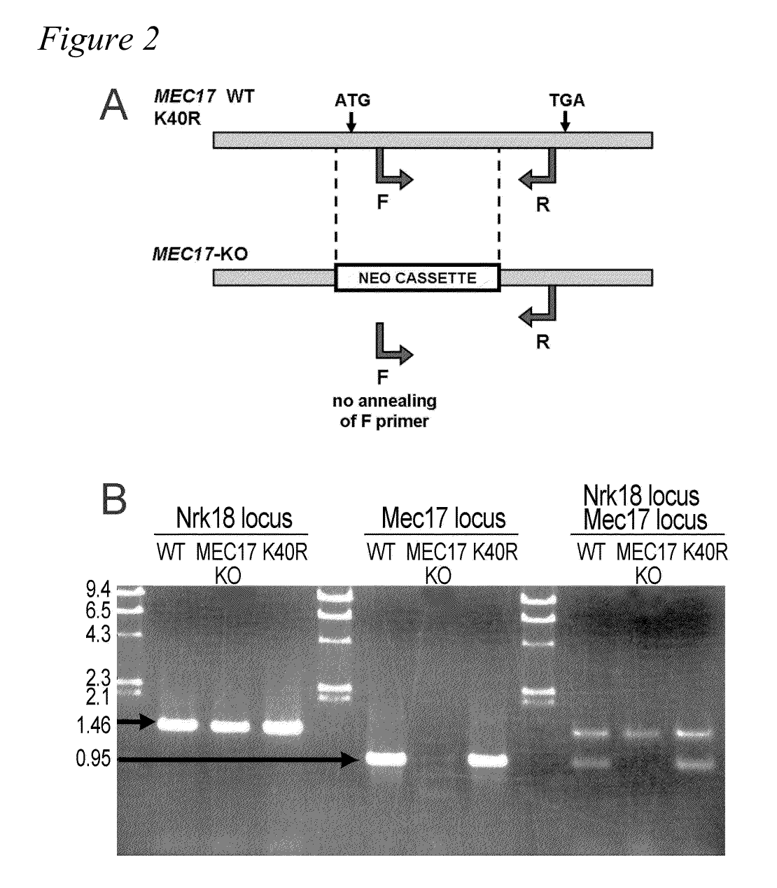

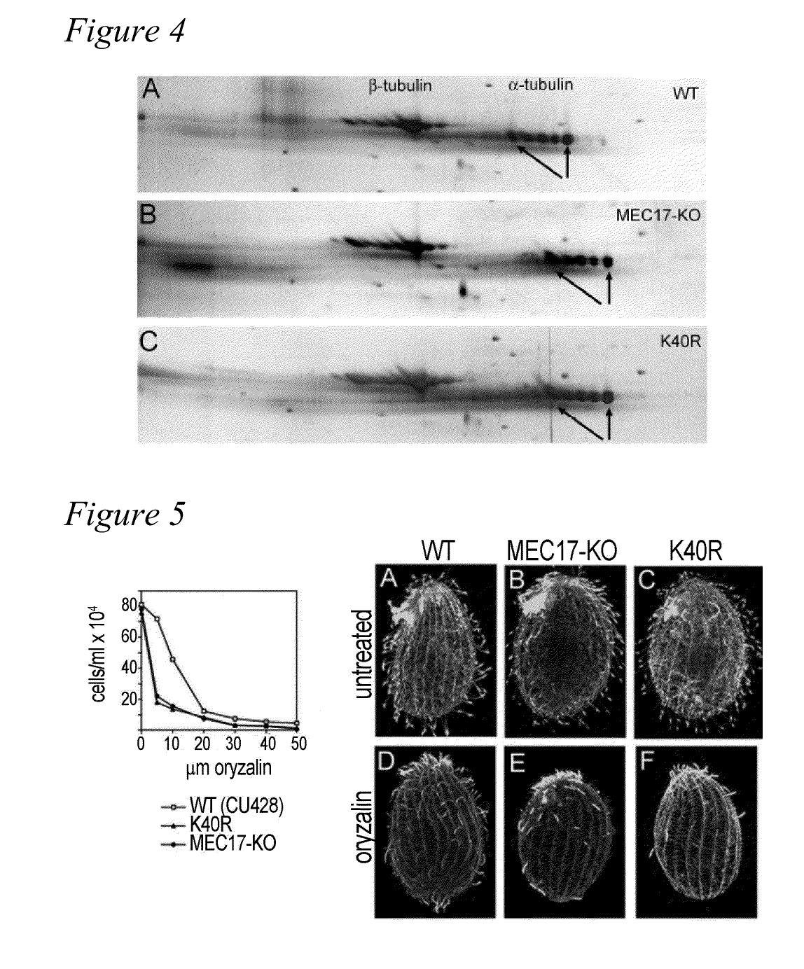

MEC-17 homologs are present in most eukaryotes with exception of fungi and plants (FIG. 1). We used DNA homologous recombination to disrupt the gene encoding MEC-17, MEC17, in the ciliate Tetrahymena thermophila (FIG. 2). Immunofluorescence with 6-11 B-1, a monoclonal antibody (mAb) that is specific for acetyl-K40 on α-tubulin (LeDizet and Piperno, 1991 Meth. Enzymol. 196:264-274) showed a marked loss of acetyl-K40 in Tetrahymena cells lacking MEC17 (MEC17-KO) (FIG. 3a-c). Western blots with 6-11 B-1 mAb showed a nearly complete loss of acetyl-K40 α-tubulin in MEC17-KO cells, comparable to cells carrying a K40R substitution in α-tubulin (FIG. 3g,h). Consistently, 2D SDS-PAGE showed that MEC-17-KO α-tubulin isoforms are more basic than wild-type isoforms (FIG. 4). On a western blot with pan-acetyl-K antibodies bands corresponding to a-tubulin and its proteolytic fragments were missing in the MEC-17-KO and K40R cell extracts, while a few non-tubulin bands (including histones) were present (FIG. 3g,h). In wild-type cells analyzed by immunofluorescence, the pan acetyl-K antibodies strongly labeled microtubules and nuclei (FIG. 3d). In the MEC-17-KO and K40R cells, acetyl-K was not detected on microtubules, but nuclei remained labeled (FIG. 3e,f). We conclude that in Tetrahymena, α-tubulin is the major if not the only substrate of MEC-17-dependent K acetylation.

The MEC-17-KO Tetrahymena cells had a normal growth rate. However, the MEC-17-KO cells grew more slowly than wild type on medium with the microtubule depolymerizing compound oryzalin. In MEC-17-KO cells treated with oryzalin, most axonemes depolymerized or were shorter than similarly-treated wild-type cells (FIG. 5). Conversely, the MEC-17 KO cells grew faster than wild-type cells in medium with paclitaxel, a microtubule-stabilizing drug (FIG. 3i). This drug phenotype is consistent with an increase in dynamics of microtubules in MEC17-KO cells (Barlow et al., 2002 J Cell Sci 115:3469-3478). Tetrahymena cells with K40R α-tubulin had a similar drug phenotype (FIG. 3i, FIG. 5). These observations indicate that in Tetrahymena, MEC-17 regulates the dynamics of microtubules by acetylation of K40 on α-tubulin.

MEC-17 is required for the maintenance of TRNs in C. elegans (Chalfie and Au, 1989 Science 243:1027-1033; Zhang et al., 2002 Nature 418:331-335). The W06B11.1 gene encodes a protein closely related to MEC-17 (Chalfie and Au, 1989 Science 243:1027-1033; Zhang et al., 2002 Nature 418:331-335). Using 6-11 B-1 mAb, we confirmed that C. elegans wild-type adults have a strong signal for acetylated α-tubulin in the six TRNs (Fukushige et al., 1999 J. Cell Sci. 112:395-403) (FIG. 6a). Single mec-17 or W06B11.1 mutants retained normal levels of acetylated microtubules in the TRNs (FIG. 6c,d). However, double mec-17 and W06B11.1 mutants lacked an acetyl-K40 signal in the TRNs similar to mec-12 α-tubulin mutants (FIG. 6e,f). Thus, MEC-17 and W06B11.1 are redundantly required for acetylation of K40 on MEC-12 α-tubulin. W06B11.1 or mec-17 single deletion mutants had reduced touch responsiveness, and a loss of both genes reduced the touch responsiveness further (FIG. 6g). Next, we investigated the role of MEC-17-dependent acetylatable K40 of α-tubulin. MEC-12 is the only α-tubulin with K40, and mec-12(e1607) (probable null allele; Bounoutas et al., 2009 Curr Biol 19:1362-1367) worms have greatly reduced touch responses. Using Mos1 transposon excision repair (Frokjaer-Jensen et al., 2008 Nat Genet 40:1375-1383), we integrated single transgenes encoding MEC-12 with either wild-type K40 or K40R or K40Q substitutions into the mec-12(e1607) mutant. The MEC-12-K40 transgene restored the levels of touch response to ˜80% that of wild type (FIG. 6g), while animals with either MEC-12-K40R or MEC-12-K40Q showed reduced touch response. With the limitation that the wildtype MEC-12 transgene does not fully restore touch sensation, and taking into account that mec12(e1607) mutants have a basal level of touch response, we calculate that a non-acetylatable MEC-12 is 30-33% less efficient than wild-type MEC-12. Nevertheless, animals with K40 substitutions on MEC-12, do respond to touch more frequently than animals lacking MEC-17 and W06B11.1. Thus we surmise that MEC-17 and W06B11.1 contribute to touch sensation partly by acetylating α-tubulin on K40, and through a second mechanism, likely by acetylation of a non-tubulin substrate(s).

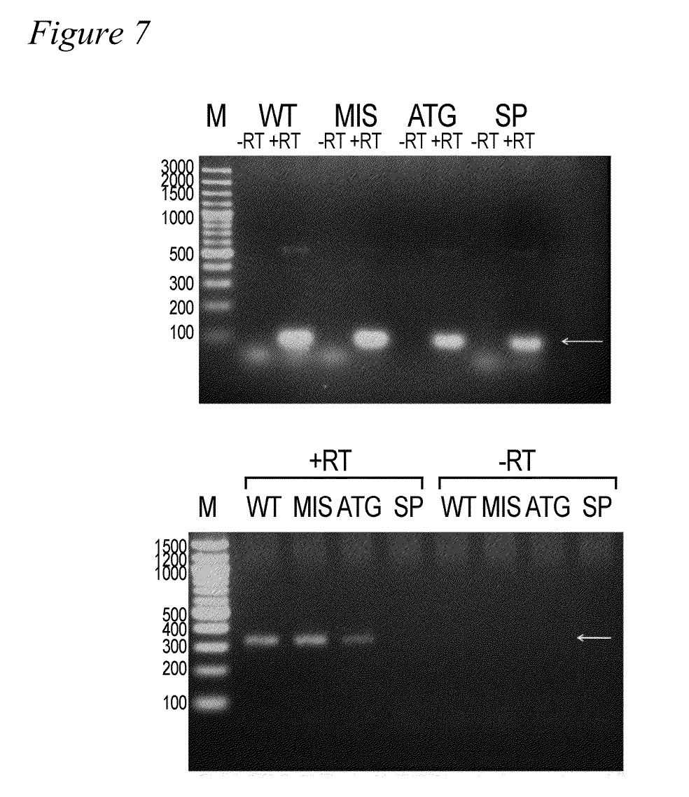

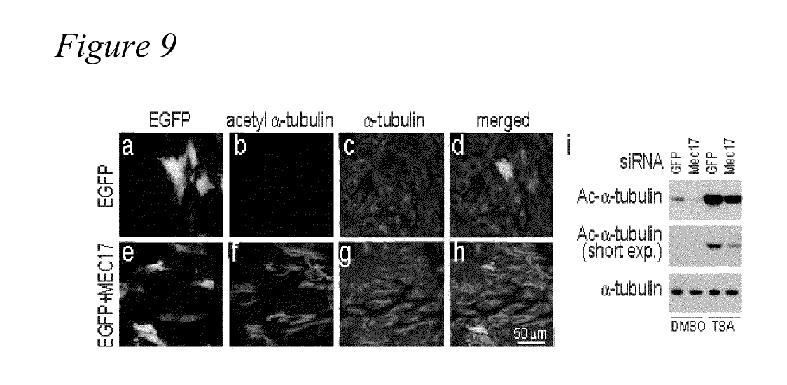

We used zebrafish to test whether MEC-17 is required for α-tubulin acetylation in vertebrates. Acetyl-K40 α-tubulin is enriched in cilia (Sun et al., 2004 Development 131:4085-4093) and axons of neurons in zebrafish (Wilson and Easter, 1991 Development 112:723-746). Zebrafish has a single MEC-17 ortholog, zgc:65893 (mec17). We injected wild-type zebrafish embryos with morpholinos (MOs) that target either the translation initiation region or a predicted splice junction of mec17. The splice junction MO caused a severe reduction in the levels of mec17 mRNA, possibly by nonsense-mediated mRNA decay (FIG. 7). Both MOs produced similar developmental defects, including curved body shape, short body axis, hydrocephalus, small head and small eyes (FIG. 8a,b). The vast majority of control embryos injected with random sequence MOs or 5 bp mismatched MOs appeared normal (Table 1 and Table 2). The mec17 morphants often did not respond or had slow startle response when probed with a needle, consistent with neuromuscular defects (Table 1 and Table 2). Immunofluorescence of wild-type embryos with 6-11 B-1 showed that acetyl-K40 carrying microtubules are abundant in the nervous system, including the brain, optical nerves, spinal cord, and axons of peripheral nerves (FIG. 8c-c″) and in cilia (FIG. 8c″). Strikingly, mec17 morphants showed a nearly complete loss of 6-11 B-1 signal in neurons (FIG. 8d-d′), but not in cilia (FIG. 8d″). The axons of primary motor neurons in the trunk were strongly labeled by the 6-11 B-1 mAb in controls but not in morphants (FIG. 8c′, d′); synaptotagmin 1 localization at synaptic termini (Fox and Sanes, 2007 J Comparative Neurology 503:280-296) indicates that the morphants do contain axons (FIG. 8e,f). Depletion of human MEC-17 (C6orf134) in HeLa cells using siRNAs reduced the levels of acetyl-K40 α-tubulin (FIG. 9i), indicating that MEC-17 is also required for α-tubulin acetylation in mammals.

TABLE 1
Frequencies of phenotypes observed at 48 hpf in fish injected with
either random sequence, or MEC17-ATG or MEC17-SP MOs.

| | 1 ng | 1 ng MEC-17 | 1 ng MEC-17 |
|---|---|---|---|
| | control MP | ATG MO | SP MP |
| Phenotype | (n = 87) | (n = 84) | (n = 106) |
| Lethality | 11% | 7% | 28% |
| Hydrocephaly | 0% | 15% | 33% |
| Short body axis | 4.5% | 35.7% | 41.5% |
| Edema of the heart | 2% | 15.4% | 13.2% |
| Small head/eyes | 3.4% | 28.5% | 47.1% |
| Reduced mobility | 5.7% | 41.6% | 23.5% |
| Paralyzed | 4.5% | 15.4% | 15% |

TABLE 2
Frequencies of phenotypes observed at 48 hpf in fish injected with
either MEC17-ATG or 5 bp mismatch control MOs.

| | 1 ng ATG MIS MO | 1 ng Mec-17 MO |
|---|---|---|
| Phenotype | (n = 77) | (n = 188) |
| Lethality | 2.5% | 3.7% |
| Hydrocephaly | 0% | 13.8% |
| Edema of the heart | 3.8% | 7.9% |
| Short body axis | 5.1% | 27.1% |
| Small head/eyes | 6.4% small heads | 79.2% small heads |
| | 1.2% small eyes | 74.4% small eyes |
| Mobility | 2.5% reduced mobility | 17.5% reduced mobility |
| | | 10.1% paralyzed |

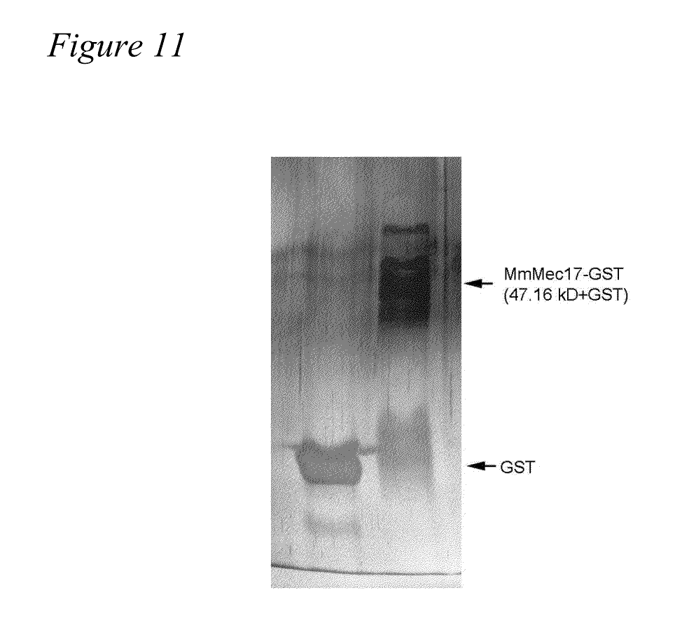

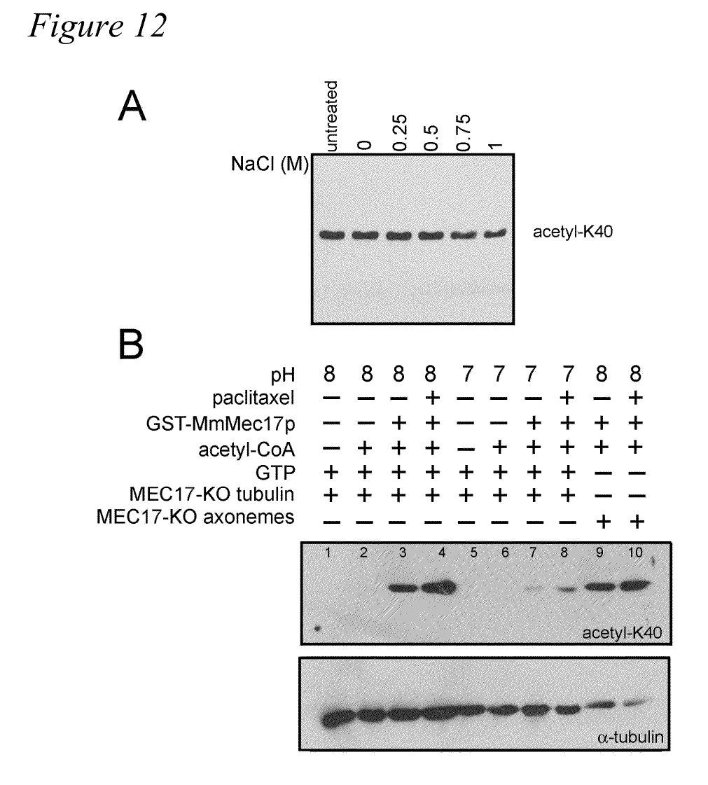

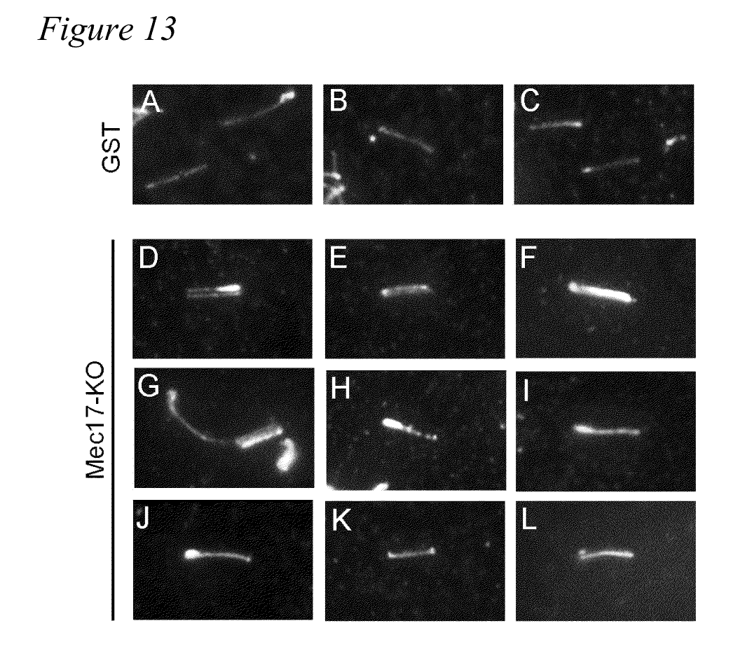

Overexpression of GFP-Mec17p in Tetrahymena greatly increased acetylation of microtubules (FIG. 3j-1). Expression of a murine homolog, MmMEC-17 (Q8K341), in PtK2 cells (which have naturally low acetyl-K40 α-tubulin), induced massive acetylation of cytoplasmic microtubules (FIG. 9a-h). The above observations indicate that either MEC-17 has intrinsic αTAT activity or is an activator of αTAT. To test whether MEC-17 alone can mediate K40 acetyltransferase activity, we established a tubulin acetylation assay using axonemes purified from Tetrahymena MEC17-KO, with acetyl-CoA. A crude GFP-Mec17p-enriched fraction (obtained from transgenic Tetrahymena) had K40 αTAT activity in vitro that was dependent on the presence of acetyl-CoA (FIG. 10a, lanes 1-3). Next, we assayed a recombinant MmMEC-17 (expressed in E. coli as a GST fusion, FIG. 11) on MEC17-KO axonemes. GST-MmMEC-17, but not GST, mediated a robust αTAT activity in vitro (FIG. 10a, lanes 4,5). To test whether the MEC-17 activity is specific to the K40 residue, we assayed GST-MmMEC-17 with axonemes from either a MEC17-KO Tetrahymena (K40 α-tubulin) or from a K40R Tetrahymena mutant (R40 α-tubulin) and used pan acetyl-K antibodies to detect acetyl modification of any K residue. GST-MmMEC-17 modified K40 axonemes (FIG. 10b, lanes 3,4) but failed to acetylate R40 axonemes (FIG. 10b, lane 5). Thus, the activity is specific to K40. Since axonemes are composed of tubulin and MAPs, there is a possibility that MEC-17 activates another protein that is an axoneme-bound αTAT. When MEC-17-KO axonemes were pretreated with 1M salt to remove MAPs, no loss of activity was detected, suggesting that MEC-17 does not require an axoneme-associated cofactor (FIG. 12a). To test whether MEC-17 has intrinsic activity, we performed an in vitro acetylation assay with highly purified tubulin obtained from Tetrahymena MEC17-KO cells (FIG. 10d). GST-MmMEC-17 mediated a robust K40 acetylation activity on purified tubulin that was comparable to the level of activity seen with axonemes (FIG. 10c, lanes 4,8). The activity of GST-MmMEC-17 was stimulated when purified tubulin was exposed to GTP to promote tubulin polymerization (FIG. 10c, lanes 3,4). Paclitaxel also stimulated MEC-17 activity, likely by promoting microtubule polymerization (FIG. 10c, lanes 8,9 and FIG. 12b). These data indicate that MEC-17 has an intrinsic α-tubulin acetyltransferase activity. The K40 residue of α-tubulin is located on the luminal surface of the microtubule (Nogales et al., 1999 Cell 96:79-88). When the MEC-17-KO Tetrahymena axonemes were subjected to in vitro acetylation by GST-MmMEC-17, the acetyl-K40 signal was observed near one or both axoneme ends, often as a decreasing gradient from the microtubule end (FIG. 13). This supports the model that MEC-17 enters the microtubule lumen from the microtubule end.

To conclude, we identified MEC-17 as an α-tubulin K40 acetyltransferase. We show that MEC-17 is important in the nervous system in both vertebrates and invertebrates. Importantly, another αTAT enzyme likely exists. MEC-17 sequences, are absent from Chlamydomonas reinhardtii, an organism that has αTAT activity (LeDizet and Piperno, 1987 Proc. Natl. Acad. Sci. USA 84:5720-5724; Maruta et al., 1986 J. Cell Biol. 103:571-579) and zebrafish embryos depleted in MEC-17 showed a dramatic loss of acetyl-K40 in neurons but not in cilia. A recent study revealed that ELP3, a conserved histone acetyltransferase, is required for normal levels of K40 acetylation and the differentiation of cortical neurons in the mouse (Creppe et al., 2009 Cell 136:551-564). However, an ELP3 expressed in insect cells and partially purified was associated with only weak αTAT activity in vitro (Creppe et al., 2009 Cell 136:551-564). Moreover, TRN microtubules remain highly acetylated in C. elegans elpc-3 mutants, which lack the sole ELP3 homolog (FIG. 6b; Solinger et al., 2010 PLoS Genet 6:e1000820; Chen et al., 2009 PLoS Genet 5:e1000561). NAT1-ARD (Ohkawa et al., 2008 Genes Cells 13:1171-1183) and NAT10 (Shen et al., 2009 Exp Cell Res 315:1653-1667) are also associated with acetylated microtubules, but it is not known whether these proteins have intrinsic αTAT activity. Thus, the identity of the second αTAT remains uncertain.

Tetrahymena cells lacking α-tubulin acetylation are resistant to paclitaxel and sensitive to oryzalin, consistent with an increase in microtubule dynamics (Barlow et al., 2002 J Cell Sci 115:3469-3478). Based on these studies, MEC-17-mediated K40 acetylation could mildly stabilize microtubules. It remains to be determined whether changes in microtubule dynamics are a direct effect of acetyl-K40 or are mediated by microtubule effector proteins. We show that in C. elegans, MEC-17 contributes to TRN function partly by acetylating K40 on MEC-12 α-tubulin and partly by other means. For example, MEC-17 could acetylate another protein, or act as a MAP, possibly inside the microtubule lumen.

### Methods Summary

To disrupt the MEC-17 gene in Tetrahymena, we used homologous DNA recombination with a fragment carrying the neo4 marker that replaced the coding region. MEC-17 was overexpressed in Tetrahymena using the MTT1 cadmium-dependent promoter. In C. elegans, MEC-12-K40, MEC-12-Q40, and MEC-12-R40 transgenes were introduced into a single site on chromosome II in the EG4322 strain. Animals homozygous for a MEC-12 transgene and homozygous for the mec-12(e1607) allele were obtained by standard crosses. All touch sensation assays in C. elegans were done using blind scoring. To deplete human MEC-17 (C6Orf134) mRNA in Hela cells, we introduced MEC-17-specific siRNAs (ON-TARGETplus pool, Dharmacon) using Oligofectamine (Invitrogen). To knockdown mec17 expression in zebrafish, MOs designed to target the MEC-17 mRNA (Open Biosystems) were injected into early embryos. ATG-MEC17 MO targets the translation initiation site of mec17 mRNA. SP-MEC17 MO targets the exon3/intron3-4 splice junction, and is expected to result in an aberrant splicing isoform of exon2 to exon 4, producing a frameshift mutation and associated protein truncation. As a negative control, we injected MO with a random sequence (oligo-25N, Gene Tools) or a 5 bp mismatch to the ATG-MEC17 MO. Live embryos were scored for phenotypes at 48 hpf. To produce a recombinant MEC-17 protein, the cDNA sequence of the murine MEC-17 (BF135007, Open Biosystems) was subcloned into pGEX-3× plasmid (GE Healthcare), expressed in BL21 E. coli cells as a GST fusion and purified using GST-Bind kit (Novagen). The in vitro acetylation assays were performed in 50 mM Tris-HCl pH 8.0, 10 mM glycerol, 0.1 mM EDTA, with purified Tetrahymena MEC-17-KO axonemes or tubulin (purified using DEAE chromatography), recombinant GST-MmMEC-17 enzyme and 10 μM acetyl-CoA. The reaction was detected by western blotting using anti-acetyl-K antibodies.

### Full Methods

### Tetrahymena

For disruption of the Tetrahymena MEC17 gene the two targeting fragments (1.4 kb of 5′ UTR and 2.0 kb of the coding region with 3′ UTR) of the MEC17 locus were designed and subcloned on the sides of the neo4 selectable cassette (Mochizuki, 2008 Gene 425:79-83). The fragments were amplified with the addition of restriction sites with the following pairs of primers: 5′-ATTGTGGGCCCTAGCATTTCTGGAAGATTCATTC-3′ (ApaI) (SEQ ID NO:1), 5′-AATACCCGGGCAATTGAATGTATGTGCTGAT-3′ (SmaI) (SEQ ID NO:2) and 5′-AAATTCTGCAGTTAGTACTTTAGAAGTGATGCT-3′ (PstI) (SEQ ID NO:3), 5′-AAATTGAGCTCTCTAGTTGACTATATTATGCATTC-3′ (SacI) (SEQ ID NO:4). The fragments were designed to remove a small part of the 5′UTR and most of the coding region and insert the neo4 resistance cassette in reverse orientation. CU428 and B2086 mating cells were biolistically transformed as described (Cassidy-Hanley et al., 1997 Genetics 146:135-147). Heterokaryons with a germline disruption of MEC17 and progeny cells homozygous for the disruption in the micronucleus and macronucleus were obtain by a heterokaryon×heterokaryon mating (Hai and Gorovsky, 1997 Proc. Natl. Acad. Sci. U.S.A. 94:1310-1315).

For overexpression of GFP-Mec17p, the coding region of MEC17 was amplified with primers 5′-ATATTACGCGTCATGGAGTTTAACTTCATCATTAATAG-3′ (SEQ ID NO:5) and 5′-ATATTGGATCCTCATTTTTTGTAGTATGTGTAGTGAT-3′ (SEQ ID NO:6) and subcloned between the MluI and BamHI sites of pMTT1-GFP plasmid (Wloga et al., 2006 Mol. Biol. Cell 17:2799-2810) and the MTT1-GFP-MEC17-BTU1 fragment was integrated into the BTU1 locus by biolistic bombardment and paclitaxel selection (Gaertig et al., 1999 Nat. Biotech. 17:462-465). The expression of GFP-Mec17p under the MTT1 promoter was induced with 2 μg/ml CdCl₂ for 2 hr.

For immunofluorescence, cells were prepared as described (Wloga et al., 2006 Mol. Biol. Cell 17:2799-2810) and stained overnight with the following antibodies: anti-acetylated K40 α-tubulin 6-11 B-1 mAb 1:200 dilution (Piperno and Fuller, 1985 J. Cell Biol. 101:2085-2094); pan anti-acetyl-K antibodies (ImmuneChem, ICP0380) at 1:150 dilution; anti-a-tubulin 12G10 mAb (Jerka-Dziadosz et al., 2001 Protist 152:53-67; Developmental Studies Hybridoma Bank at 1:25; and polyclonal anti-tubulin antibodies (SG, 1:600). To compare the levels of tubulin acetylation side-by side, wild type cells were marked by feeding for 10 min with India Ink and mixed with MEC 17-KO cells.

For western blotting studies, wild type (CU428), MEC17-KO and K40R mutant cells (Gaertig et al., 1995 J. Cell Biol. 129:1301-1310) were grown to the mid-log phase. The cytoskeletal fractions were prepared as described (Janke et al., 2005 Science 308:1758-1762) except that trichostatin A at 1 μg/ml was added to concentrated cells prior lysis. Total extracts of 2×10⁴ cells, or 5 μg of cytoskeletons per lane were used for western blotting with the following antibodies: 12G10 mAb (1:5000); 6-11 B-1 mAb (1:5000); pan anti-acetyl-K antibodies (1:300); hvl anti-histone (1:2000).

### Zebrafish

To knockdown MEC-17 expression in zebrafish, two morpholinos designed to target the MEC-17 mRNA (Open Biosystems) were injected into embryos (at 3 ng/embryo): ATG-MEC17 (5′-CATTCAGGTCGTAAGGGAAATCCAT-3′; SEQ ID NO:7) and SP-MEC17 (5′-AGAGAAAGCTATTTTACCCGTTCTG-3′; SEQ ID NO:8). ATG-MEC17 targets the translation initiation site of MEC-17 mRNA. SP-MEC17 MO targets the exon3/intron3-4 splice junction, and is expected to result in an aberrantly spliced isoform in which exon2 is joined to exon 4. The predicted transcript contains a frameshift mutation and encodes a nonsense protein. As a negative control we injected MO with a random sequence (oligo-25N, Gene Tools) or a 5 bp mismatch to the ATG-MEC17 MO (5′-CATTgAcGTCcTAAGGcAAATgCAT-3′; SEQ ID NO:9). Live embryos were scored for phenotypes at 48 hpf, or fixed processed for immunofluorescence as described (Wloga et al., 2009 Dev Cell 16:867-876). The antibodies were used in the following concentrations: 6-11 B-1 mAb (1:1000), Znp-1 anti-synaptotagmin 1 mAb (1:100). After incubation with secondary antibodies (Zymed) overnight at 4° C. (1:500) embryos were mounted in 100 mg/ml of DABCO (Sigma-Aldrich) in PBS and viewed in a Leica TCS SP confocal microscope. Live zebrafish morphants shown in videos S1-S3 were recorded using on the Zeiss Stemi SV11 Apo microscope and a SPOT FLEX camera (Diagnostic Instruments Inc.) at 12 frames per second.

For RT-PCR, mRNA was isolated from 70 embryos 24 hpf using TRIzol reagent (Invitrogen, Carslbad, Calif.), and total cDNA was synthesized using iScript cDNA synthesis kit (Bio-Rad Laboratories, Hercules, Calif.). The sequences of primers used to amplify the mec17 cDNA were: MEC17EX1F 5′-GGTCGGAAAGCGCATGGGAG-3′ (SEQ ID NO:10) and MEC17EX5R2 5′-GAAGTCGAAGAGCTCTGAGCC-3′ (SEQ ID NO:11). The forward primer binds to exon 1 and the reverse primer binds to exon 5 (the splice blocking morpholino binds to the junction between exon 3 and the intron 3-4). For the control amplification of β-actin cDNA, we used the following primers: 5′-GATTCGCTGGAGATGATG-3′ (SEQ ID NO:12) and 5′-GTCTTTCTGTCCCATACCAA-3′ (SEQ ID NO:13).

C. elegans.

C. elegans strains were cultured as described (Brenner, 1974 Genetics 77:71-94). The following strains were used: N2, wild type; CB1607, mec-12(e1607) III; RB1696, mec-17(ok2109) IV; RB1869, W06B11.1(ok2415) X; ET389, mec-17(ok2109) IV, W06B11.1(ok2415) X; VC1937, elpc-3(gk2452) X; ET431, mec-12(e1607) III, ekSi1 [Pmec-12::MEC-12::3′UTR mec-12] II; ET432, mec-12(e1607) III, ekSi2[Pmec-12::MEC-12(K40Q)::3′UTR mec-12] II; ET433, mec-12(e1607) III, ekSi3[Pmec-12::MEC-12(K40R)::3′UTR mec-12] II; EG4322, ttTi5605; unc-119(ed3).

A MEC-12 plasmid, pMEC-12, was constructed using a MEC-12 cDNA and genomic sequence as described (Fukushige et al., 1999 J. Cell Sci. 112:395-403). Using overlapping PCR, we mutated the K40 codon to either a Q or R codon on pMEC-12 along with silent substitutions creating restriction sites (PvuI site for R40 plasmid and HindIII site for Q40 plasmid) and confirmed the mutagenesis by sequencing the entire plasmids. The K40, R40 or Q40 derivatives of pMEC-12 were used to prepare targeting plasmids for Mos-SCI (Frokjaer-Jensen et al., 2008 Nat Genet 40:1375-1383) as follows. pCFJ151 is a plasmid vector designed to target fragments into the ttTi5605 locus on chromosome II (Frokjaer-Jensen et al., 2008 Nat Genet 40:1375-1383). A 1.7Kb fragment pMEC-12 comprising a part of the MEC-12 cDNA sequence and 3′UTR was amplified using the primers 5′-ATTATGTTTAAACCAAGCTCGAGTTCTCCATC-3′ (SEQ ID NO:14; PmeI site is underlined and XhoI site shown in bold) and 5′-AATTATGATCACAGCAAAGGATTCAAGGCTC-3′ (SEQ ID NO:15; BclI site is underlined), digested with BclI and BglII and inserted into a modified pCFJ151 lacking its original XhoI site (as a result of earlier XhoI digestion, blunting and religation). The resulting plasmid was digested with PmeI and XhoI and used for insertion of a 5.7 Kb of 5′UTR and a part of cDNA amplified from either pMEC-12-K40, or p-MEC-12Q40 or pMEC12-R40 using primers 5′-ATAATGTTTAAACCGGCGAGAAGAGCTATCAA-3′ (SEQ ID NO: 16; with PmeI site underlined) and 5′-AATTTGGAGAACTCGAGCTTGGCC-3′ (SEQ ID NO: 17; with XhoI underlined). The resulting plasmids: pCFJ151-MEC-12-K40, pCFJ151-MEC-12-Q40 and pCFJ151-MEC-12-R40 were used for introduction of single copy MEC-12 transgenes into a site on chromosome II of the EG4322 strain and integrant animals were identified as described (Frokjaer-Jensen et al., 2008 Nat Genet 40:1375-1383). Strains homozygous one of the three MEC-12 transgene types and homozygous for the mec-12(e1607) probable null allele (Bounoutas et al., 2009 Curr Biol 19:1362-1367) were obtained by standard crosses. All touch assays were done using blind scoring. To determine the touch response level, 30 L4 larvae were isolated on a 5 cm 1×NGM OP50 seeded plate and adult animals were scored for touch responses after 24 hr. Each animal was touched 10 times by moving an eyebrow hair across the body below the anterior and posterior ends. The level of touch response was calculated as an average number of responses per 10 touches.

For immunofluorescence, animals were made permeable by the ‘freeze-crack’ method, followed by methanol and acetone fixation (10 min at −20° C. for each) (Miller and Shakes, 1995 Method Cell Biol 48:365-394), and probed with the primary antibody (6-11 B-1 mAb, 1:500 dilution) and the secondary antibody (anti-mouse rhodamine (Cappel, 1:50). Animals were observed with a Zeiss Axioskop microscope equipped for differential interference contrast (DIC) and fluorescence microscopy. Images were captured with a Hamamatsu ORCA-ER digital camera with Openlab 5.0.2 software (Improvision). Images were processed using Adobe Photoshop CS2. Matched images were taken with the same exposure and processed identically. There was no statistical significance between the acetyl-K40 signal intensities over TRNs (after adjacent background subtraction) in wild type, mec-17, and W06B11.1 single mutant strains (241±117; 294±111; and 252±162 arbitrary pixel intensity units, respectively) (FIG. 6a,c,d). The signal intensity in mec-12 mutants or the double mutant mec-17; W06B11.1 could not be determined because there was no detectable TRN signal to measure (FIG. 6e,f).

### Mammalian Cells

PtK2 rat kangaroo kidney epithelial cells were grown in DMEM medium supplemented with 10% fetal calf serum, 2 mM L-glutamine and an antibiotics mix at 37° C. with 5% CO₂. For transfection, cells were grown in 24-well plates to 80-90% of confluency, and transfected with Lipofectamine 2000 (Invitrogen) according to manufacturer instructions, using either 20 ng of pEGFP-N1 plasmid (Clontech) plasmid alone or 20 ng of pEGFP-N1 and 800 ng of pCMV-SPORT6-mmMEC17 (Open Biosystems, MMM1013-7510854) for 16-20 h. Following transfection, cells were grown for 48 hr, split onto coverslips and grown for another 24 hrs and subjected to immunofluorescence. Coverslips with cells were rinsed with PBS, fixed in 4% paraformaldehyde in PBS for 12 min and permeabilized in 0.5% Triton-X-100 in PBS for 15 min. After permeabilization, coverslips were incubated in 3% BSA in PBS for 10 min and incubated in primary antibodies diluted in 3% BSA in PBS (6-11 B-1 anti-acetyl-K40 at 1:300 1:10 and polyclonal anti-α-tubulin (Sigma-Aldrich) 1:10 for 2 hr. After 3×5 min washes with PBS cells were incubated in secondary antibodies: anti-mouse IgG-Cy3 in 3% BSA in PBS at 1:100 for 1 hr, washed 3 times and mounted with 100 mg/ml DABCO (Sigma) in PBS and viewed in a Leica TCS SP confocal microscope.

To deplete human MEC-17 (C6Orf134) mRNA in Hela cells, we used ON-TARGETplus siRNAs from Dharmacon as a pool of four siRNAs; the sequences are as follows:

### 5′-GUAGCUAGGUCCCGAUAUA-3′ (#1; SEQ ID NO:18);

### 5′-GAGUAUAGCUAGAUCCCUU-3′ (#2; SEQ ID NO:19);

### 5′-GGGAAACUCACCAGAACGA-3′ (#3; SEQ ID NO:20);

5′-CUUGUGAGAUUGUCGAGAU-3′ (#4; SEQ ID NO:21). GFP siRNA, 5′-GCUGACCCUGAAGUUCAUCUGdTdT-3′ (SEQ ID NO:22; Invitrogen) was used as a negative control. HeLa cells were grown as above and transfected with 100 nM of siRNAs (in the pool, each siRNA was at 25 nM) using Oligofectamine (Invitrogen) transfection reagent in accordance with the manufacturers' instructions. Transfections were performed three times sequentially, followed by subculturing into the new wells. Fifty hr after the first transfection, 300 nM of Trichostatin A in DMSO or the same volume of DMSO were added to the cell cultures, and cells were grown for another 7 hr. Cells were collected and lysed with boiling Laemmli loading buffer containing 2.5% SDS. Lysates of equal number of cells were analyzed using SDS-PAGE/western blot with mouse antibody against acetylated a-tubulin (6-11B-1, Sigma), 1:1000, and mouse anti-α-tubulin antibody (DM1A, Sigma), 1:10,000.

### Substrates for In Vitro Tubulin Acetylation.

To prepare MEC 17-KO axonemes, Tetrahymena cells were grown to the mid-log phase and deciliated by pH shock (Wloga et al., 2008 Eukaryot Cell 7:1362-1372). Cilia were suspended in 1 ml of 1% NP-40 in the axoneme buffer (30 mM HEPES, 20 mM potassium acetate, 5 mM MgSO₄, 0.5 mM EDTA, pH 7.6) with Complete protease inhibitors (Roche). After 1-2 min on ice, axonemes were collected by centrifugation (20,000×g, 15 min, 4° C.), suspended in the in vitro acetylation reaction buffer (50 mM Tris HCl, pH 8.0, 10% glycerol, 0.1 mM EDTA, 1 mM DTT) with protease inhibitors and stored at −80° C.

Total tubulin was purified from the MEC17-KO strain of Tetrahymena using a protocol modified after Yakovich and colleagues (Yakovich et al., 2006 Exp Parasitol 114:289-296). Tetrahymena cells (2×10⁹) were suspended in 40 ml of PME+P buffer (0.1 M Pipes pH 6.9, 1 mM MgCl₂, 1 mM EGTA, 1 mM benzamidine, 0.5 mM phenylmethylsulfonyl fluoride, and 25 μg/ml leupeptin) on ice. Cells were sonicated on ice using a Sonic Dismembrator Model 100 (Fischer Scientific) with ten 30 sec bursts at 25 W with a 2 min cooling interval between each burst. The lysate was incubated on ice for 30 min and centrifuged at 40,000×g for 30 min at 4° C. The supernatant was filtered through glass wool and loaded into a 10 ml column DEAE-Sepharose Fast Flow Matrix (GE Healthcare, earlier equilibrated with two volumes of PME+P) at a rate of 2.5 ml/min using a peristaltic pump. The column was washed with two column volumes of PME+P and followed by four column volumes PME+P with 0.1 M KCl, 0.25 M glutamate pH 6.9. Tubulin was eluted with two column volumes PME+P and 0.3 M KCl, 0.75 M glutamate pH 6.9. Two and half ml fractions were collected. Fractions 6 through 8 were pooled and supplemented with 10 mM MgCl₂, 8% DMSO (v/v), and 2 mM GTP. The tubulin-rich pooled fraction was incubated at 37° C. for 60 min to induce microtubule assembly and centrifuged at 50,000×g at 30° C. for 30 min. The pellet consisting of microtubules was rinsed once with warm PME (˜37° C.), and suspended in ˜1.5 ml of ice-cold PME. The pellet was solubilized by sonication (thirty ˜5 s bursts at 10 W). The tubulin solution was incubated on ice for 30 min, and centrifuged 50,000×g at 4° C. for 30 min. The supernatant containing highly purified dimeric tubulin was stored at −80° C. in 50 μl aliquots.

To polymerize tubulin, 100 μl of purified Tetrahymena MEC-17-KO tubulin (5.5 mg/ml) in PME buffer (100 mM Pipes, 1 mM MgCl₂, 1 mM EGTA, pH=6.9) was combined with 80 μl of BRB80 buffer (80 mM PIPES, 1 mM MgCl₂, 1 mM EGTA, pH 6.8), 2 μl of 100 mM GTP and 1 μl of 0.2 M DTT and incubated for 1 hr at 37° C. Microtubules were collected by centrifugation (13,000 rpm, 10 min at room temperature) and the pellet was suspended in the acetylation reaction buffer (see above).

### Expression and Purification of MEC-17 Enzymes.

Tetrahymena cells with a GFP-Mec17p encoding transgene under MTT1 promoter were grown without paclitaxel to the density 2×10⁵ cells/ml (25 ml) and overexpression was induced by incubation with 2.5 μg/ml of CdCl₂ for 3 hr. The cells were collected by centrifugation, washed with Tris-HCl buffer, pH7.5, suspended in the cold in vitro acetylation buffer with protease inhibitors and gently homogenized on ice using a Dounce tissue grounder. The homogenate was centrifuged for 20 min at 20,000×g at 4° C. and the supernatant stored in aliquots at −80° C.

To express recombinant MEC-17 proteins, the pCMV-SPORT6 plasmid containing a full cDNA sequence of the murine MEC-17 ortholog (BF135007, Open Biosystems) was used as template for amplification of the coding regions with primers: 5′-AAATTGAGCTCTGGAGTTCCCGTTCGATGTGGAT-3′ (SEQ ID NO:23) and 5′-AATAGAATTCCCGCGGACTAAGCTTTGGCCATGGTTACC-3′ (SEQ ID NO:24). The fragment was subcloned into pGEX-3× expression vector (GE Healthcare). The E. coli BL21 cells carrying either pGEX-3× (GST) or pGEX-3X-MmMEC-17 (GST-MmMEC-17) plasmids were grown in 3 ml cultures of LB medium with ampicilin (50 μg/ml concentration) overnight at 37° C. with shaking A 1.5 ml of culture was transferred into 25 ml of LB medium, IPTG was added to 1 mM final concentration and bacteria were grown for 2.5-3 hrs at 37° C. with shaking Bacteria were collected by centrifugation (6000×g, 10 min), washed with 25 ml of cold washing buffer (20 mM Tris-HCl, pH 8.0, 0.2M NaCl, 10% glycerol, 2 mM EDTA) and centrifuged as above. Bacteria were suspended in 1 ml of washing buffer supplied with 5004 β-mercaptoethanol, 0.5 mM PMSF, 10 μg/ml leupeptin, 5 μg/ml DNAse I, 10 μg/ml RNAse A, 1 mg/ml lysozyme, subjected to 2-3 rounds of freezing at −80° C. (20-30 min each) followed by thawing on ice, followed by 10 passages through a syringe with an 18 gauge needle. The homogenate was centrifuge at 16,000×g for 20 min at 4° C. and GST-tagged recombinant proteins were purified with GST-Bind Kit (Novagen) according to manufacturer instructions. The recombinant proteins were stored aliquoted at −80° C.

### In Vitro Tubulin Acetyltransferase Assay

The assays were performed in a buffer that was used earlier for histone acetyltransferases containing 50 mM Tris-HCl pH 8.0, 10 mM glycerol, 0.1 mM EDTA, 1 mM DTT (Kuninger et al., 2007 J Biotech 131:253-260) in 50 μl volumes that included 5 μl of purified Tetrahymena MEC-17-KO axonemes or tubulin, 10 μl of GFP-Mec-17p supernatant or purified GST-MmMEC-17 enzyme and 0.5 μl of 1 mM acetyl-CoA). Samples were incubated for 60-90 min at 28° C. The reaction was stopped by addition of 5×SDS sample buffer and heating for 5 min at 96° C. Proteins from 10 μl of samples were separated on 10% SDS-PAGE gel and transferred onto nitrocellulose and processed with 6-11 B-1 anti-acetylated K40 α-tubulin mAb (1:15,000) or pan anti-acetyl-K antibodies (1:500) or anti-a-tubulin antibodies (12G10, 1:10,000), as described above.

### Example III

Microtubules are fibers made of polymerized dimers of α- and β-tubulin. Microtubules are essential for many cellular functions, including the long range intracellular transport that is mediated by motor proteins. Commonly, specific cellular cargoes (protein complexes or organelles) are transported by motor proteins into specific locations along subsets of microtubules. One striking example is the neuron, where cargoes are moved from the cell body either into the dendrite or axon projections. The principles that govern the selective transport and other localized phenomena on the surface of microtubules are not well understood. We have been testing a model hypothesizing that subsets of microtubules are functionally adapted by post-translational modifications (PTMs) of tubulin subunits. Acetylation of lysine 40 (K40) on α-tubulin is a conserved PTM (L'Hernault and Rosenbaum, 1983 J Cell Biol 97:258-263) that in neurons is more abundant on microtubules in the axon as compared to dendrites (Witte et al., 2008 J Cell Biol 180:619-32; Shea, 1999 Brain Res Bull 4: 255-61). We have shown that kinesin-1, an axon-targeted motor, preferentially binds to microtubules marked by acetyl-K40 (Reed et al., 2006 Curr Biol 16:2166-2172). Thus, acetyl-K40 on α-tubulin could be a key determinant in cell polarization. The enzyme responsible for K40 acetylation is unknown, limiting study of the function of this tubulin PTM. We report here an identification of a protein that is required for K40 α-tubulin acetylation in Tetrahymena and zebrafish. This protein is an ortholog of MEC-17, a protein required for the function of touch receptor neurons in C. elegans (Zhang et al., 2002 Nature 418:331-5). MEC-17 mediates acetylation of α-tubulin in vitro and is the long-sought a-tubulin K40 acetyltransferase (see Examples I and II). We expect that MEC-17 affects neuronal differentiation and neuronal microtubules in vivo.

Microtubule PTMs.

Tubulin is modified by several conserved PTMs. The acetylation of K40 on α-tubulin is unusual as it is the only PTM that is located inside the microtubule lumen. Due to its localization, K40 acetylation could regulate the luminal activities whose functions remain obscure (Garvalov et al., 2006 J Cell Biol 174:759-65; Nicastro et al., 2006 Science 313:944-8), but this PTM can also affect the surface of microtubules (Reed et al., 2006 Curr Biol 16:2166-2172).

K40 α-Tubulin Acetylation is a Marker of “Old” Microtubules.

Acetylation of the a-amino group of K40 on α-tubulin was discovered on the flagellar axoneme microtubules in Chlamydomonas (L'Hernault and Rosenbaum, 1985 Biochem 24:473-478; Greer et al., 1985 J Cell Biol 10:2081-2084). A monoclonal antibody (mAb), 6-11 B-1, which recognizes specifically acetyl-K40 of α-tubulin (LeDizet and Piperno, 1991 Meth Enzymol 196:264-274), revealed that this PTM is conserved from protists to mammals, and in addition to axonemes, also marks the spindle and some cytoplasmic microtubules (Piperno and Fuller, 1985 J Cell Biol 101:2085-2094). K40 is located within a loop connecting the H1 and S2 domains of α-tubulin, which is exposed to the lumen (Nogales et al., 1999 Cell 96:79-88). Acetyl-K40 has been characterized as a marker of the age of microtubules. Typically, animal cells contain two populations of microtubules: short-lived microtubules that turnover rapidly by cycles of polymerization-depolymerization (dynamic instability), and long-lived or “stable” microtubules, that play a role in cell polarization and serve as tracks for long-range transport (Schulze and Kirschner, 1987 J Cell Biol 104:277-88; Saxton et al., 1984 J Cell Biol 99:2175-2186; Wen et al., 2004 Nat Cell Biol 6:820-30; Cai et al., 2007 Biophys J 92:4137-44). The stable microtubules are enriched in acetyl-K40 (Piperno et al., 1987 J Cell Biol 104:289-302; Webster and Borisy, 1989 J Cell Sci 92:57-65). A partially purified activity of α-tubulin K40 acetyltransferase (αTAT) prefers microtubules over unpolymerized tubulin as an acetylation substrate (Maruta et al., 1986 J Cell Biol 103:571-579). It appears that αTAT acts relatively slowly after microtubule assembly, and as a consequence, acetylation accumulates on the long-lived microtubules (Wen et al., 2004 Nat Cell Biol 6:820-30). Acetylation of microtubules using crude αTAT did not detectably change the bulk rates of polymerization and depolymerization of microtubules (Maruta et al., 1986 J Cell Biol 103:571-579). However, recent studies suggest that acetyl-K40 not only marks, but also stabilizes microtubules (see below).

K40 Acetylation has its Own Microtubule-Stabilizing Activity.

Acetyl-K40 is abundant on highly stable microtubules that form the axonemes of cilia in protists (Greer et al., 1985 J Cell Biol 10:2081-2084; Maruta et al., 1986 J Cell Biol 103:571-579). However, the PTM is not essential in protists. Ectopic expression of a nonacetylatable K40R α-tubulin in Chlamydomonas did not noticeably change the phenotype (Kozminski et al., 1993 Cell Motil Cytoskel 25:158-170). Originally we reported that in Tetrahymena, a replacement of the a-tubulin gene with a K40R variant did not affect the gross phenotype (Gaertig et al., 1995 J Cell Biol 129:1301-1310). However, we recently re-examined Tetrahymena expressing the K40R α-tubulin, and found that these cells display increased resistance to a microtubule stabilizing drug, paclitaxel, and increased sensitivity to a microtubule depolymerizer, oryzalin (see Example II and FIG. 3i). Similar drug phenotypes (for other tubulin mutations) are associated with reduction in ratio of polymerized/unpolymerized tubulin, consistent with a shift to a more dynamic population of microtubules (Barlow et al., 2002 J Cell Sci 115:3469-78; Kamath et al., 2005 J Biol Chem 280:12902-7). Thus, in vivo, acetyl-K40 could make microtubules more stable.

Two conserved enzymes, HDAC6 and SIRT2, function as K40 α-tubulin deacetylases in mammals (Hubbert et al., 2002 Nature 417:455-458; Matsuyama et al., 2002 EMBO J 21:6820-31; North et al., 2003 Mol Cell 11:437-44; Zhang et al., 2003 EMBO J 22:1168-79). HDAC6, a member of the family of histone deacetylases, is a cytoplasmic enzyme that deacetylates α-tubulin (Hubbert et al., 2002 Nature 417:455-458), HSP90 (Kovacs et al., 2005 Mol Cell 18:601-7) and cortactin (Zhang et al., 2007 Mol Cell 27:197-213). SIRT2, a protein related to SIR2, an NAD-dependent histone deacetylase of S. cerevisiae, also deacetylates α-tubulin (North et al., 2003 Mol Cell 11:437-44) and p300 (Han et al., 2008 Biochem Biophys Res Comm 375:576-80). The consequences of loss of HDAC6, a major K40 deacetylase (Zhang et al., 2008 Mol Cell Bio 28:1688-701), support a role of K40 acetylation in the dynamics of microtubules. Chemical inhibition of HDAC6 leads to an accumulation of acetylated microtubules (as expected), but also increases the ratio of polymerized/soluble tubulin and makes microtubules more resistant to a depolymerizing drug (Matsuyama et al., 2002 EMBO J 21:6820-31). Consistent with this, over-expression of HDAC6, decreases the levels of acetyl-K40 and makes microtubules more sensitive to depolymerizing treatments (Matsuyama et al., 2002 EMBO J 21:6820-31). K40 acetylation could play a role in lamellipodia-based cell motility. Depletion of HDAC6 in fibroblasts increases the area of cell adhesion and reduced cell motility (Tran et al., 2007 J Cell Sci 120:1469-79), and overexpression of HDAC6 increased the rate of cell motility (Hubbert et al., 2002 Nature 417:455-458). On the other hand, HDAC6 also deacetylates cortactin, an actin regulator (Zhang et al., 2007 Mol Cell 27:197-213) and can sometimes act independently of its deacetylase activity (Kawaguchi et al., 2003 Cell 115:727-38). However, in HDAC6-deficient fibroblasts, the hyper-acetylated microtubules are less dynamic (Tran et al., 2007 J Cell Sci 120:1469-79). In wild type motile mammalian cells, the ends of dynamic microtubules repeatedly target the focal adhesion sites and regulate the adhesion/de-adhesion cycle (Kaverina et al., 1998 J Cell Biol 142:181-90). Thus, K40 acetylation/deacetylation may be required for proper dynamics of microtubules, which in turn could regulate the adhesion site turnover. All these studies indicate that acetyl-K40 increases the stability of microtubules. However, one study does not agree with this conclusion: inhibition of HDAC6 led to an accumulation of acetyl-K40, but did not affect the levels of detyrosinated α-tubulin, another marker of stable microtubules (Palazzo et al., 2003 Nature 417:455-458). Thus, the stabilizing effect of acetyl-K40 can be uncoupled from accumulation of certain markers of stable microtubules.

Stabilization of Microtubules is Important During Neuronal Differentiation.

Even a subtle change in the dynamics of microtubules could have consequences during organismal development. Several studies indicate that a strict regulation of the dynamics of microtubules is a key requirement for differentiation of neurons, and that acetyl-K40 could play a role in this process. Undifferentiated neural cells have low levels of acetyl-K40 on microtubules. When neural cells are induced to differentiate in vitro, and over the course of several days extend projections (neurites), the stability of neurite microtubules increases, along with an increase in the levels of acetyl-K40 (Shea, 1999 Brain Res Bull 4: 255-61; Lim et al., 1989 J Cell Biol 109:253-63; Black and Keyser, 1987 J Neurosci 7:1833-42; Black et al., 1989 J Neurosci 9:358-68; Watson et al., 1990 J Neurosci 10:3344-52). Moreover, the differential stability of microtubules could contribute to the functional distinction between the two types of neurites: axons and dendrites. Typically, in an early stage of differentiation, a mammalian neuron has several short neurites of about equal length, but eventually, one of these neurites elongates and differentiates into an axon, while the remaining neurites become dendrites (Witte and Bradke, 2008 Curr Opin Neurobiol 18:479-87). In cultured hippocampal neurons, the axon has more stable microtubules and higher levels of acetyl-K40, as compared to dendrites (Witte et al., 2008 J Cell Biol 180:619-32). Within the growing or fully elongated axon, the acetyl-K40 tubulin is enriched within the distal segment. Moreover, the distal axonal segment appears to carry a “molecular memory” of its axonal identity. When an axon is experimentally severed within the distal segment, it can regenerate and maintain its axonal identity (based on the presence of Tau and absence of MAP2, the axonal and dendritic MAP markers, respectively). However, when the axon is severed within the proximal segment, one of the dendrites undergoes re-differentiation and elongates into an axon, while accumulating acetyl-K40 (Gomis-Ruth et al., 2008 Curr Biol 18:992-1000). Remarkably, multiple axons differentiate when neurons are treated with the microtubule-stabilizing drug, paclitaxel (Witte et al., 2008 J Cell Biol 180:619-32). Thus, it appears the stable (K40-acetylated) microtubules could be the carrier of axonal identity.

How the K40 acetylation of α-tubulin influences microtubules at the molecular level is an important question. One model is that acetyl-K40 regulates interactions of microtubules with motors. For an axon to grow and keep its identity there is a need for selective transport of components from the cell body. Due to its enrichment in the axon, tubulin acetylation could stimulate the motor-based transport either by promoting the entry of motors from the cell body into the axon, or by helping to sustain the transport within the axon. Kinesin-1 is a motor that preferentially accumulates in the axon (Nakata and Hirokawa, 2003 J Cell Biol 162:1045-55). In an unpolarized neuron, ectopic kinesin-1 marks a single neurite before it elongates into an axon (Witte et al., 2008 J Cell Biol 180:619-32; Jacobson et al., 2006 Neuron 49:797-804). Also, an unpolarized neuron usually has a single neurite that has elevated levels of acetyl-K40, presumably the same one that later differentiates into an axon (Witte et al., 2008 J Cell Biol 180:619-32). Remarkably, Reed and colleagues (with a contribution from our lab) showed that in vitro, kinesin-1 binds with higher affinity to microtubules that have acetyl-K40 (as compared to R40 microtubules) (Reed et al., 2006 Curr Biol 16:2166-2172). Acetylated microtubules also support faster kinesin-1-based motility on microtubules in vitro, and inhibitors of HDAC6 stimulate the transport of kinesin-1 cargo, JIP1, into neurite extensions in vivo (Reed et al., 2006 Curr Biol 16:2166-2172). Dompierre and colleagues extended this study by showing that chemical acetylation of microtubules enhances the binding of both recombinant kinesin-1 and cytoplasmic dynein (the anterograde and retrograde axonal motors, respectively) (Dompierre et al., 2007 J Neurosci 27:3571-83). Thus, acetyl-K40 could be a determinant that promotes the movement of motor proteins into the axon, or could help in sustaining transport within the axon.

At first glance, it appears not to make sense that a luminal PTM affects a motor on the outside surface of microtubules. However, a change inside the lumen can affect the microtubule surface by long-range conformational rearrangements. For example, a mutation of luminal G56 on α-tubulin inhibits the assembly of outer dynein arms on the surface of sperm axonemes (Raff et al., 2008 Curr Biol 18:911-4). Thus, this work could provide critical reagents (stoichiometrically acetylated microtubules) that in the future could be used to assess the structural consequences of the acetyl-K40 modification.

In light of the potential effects of acetyl-K40 marks on motor proteins, it is possible that the apparent polymer-stabilizing effects of this PTM in vivo, which we discussed earlier, are indirect. For example, K40 acetylation of the microtubule track could recruit motor proteins, which then bring increased quantities of microtubule-stabilizing factors into specific cellular locations. Alternatively, the PTM could have a microtubule-stabilizing effect that is independent of its effect on motors, for example by regulating the binding of structural MAPs (Tau, MAP2) that decorate the surface of microtubules.

Stabilization of Neural Microtubules and K40 Acetylation has Medical Relevance.

Neurons of Alzheimer disease (AD) patients have disorganized microtubules and lower levels of acetyl-K40 (Butler et al., 2007 Eur J Pharm 562:20-7). In a brain slice model of AD, paclitaxel partially rescues the AD phenotype (including accumulation of protein aggregates), suggesting that insufficient stability of microtubules contributes to AD (Butler et al., 2007 Eur J Pharm 562:20-7). Thus, acetyl-K40 is at the least a useful marker of AD, and could contribute to AD pathology. Recently, Dompierre and colleagues uncovered a link between acetyl-K40 and another neurodegenerative disorder, Huntington's disease (HD) (Dompierre et al., 2007 J Neurosci 27:3571-83). In HD, mutations in huntingtin (htt) affect the microtubule-based transport of vesicles containing the brain-derived neurotrophic factor, BDNF (Gauthier et al., 2004 Cell 118:127-38). Remarkably, in cell lines derived from a mouse HD model, the deficiency in the BDNF vesicle transport was corrected by HDAC6 inhibitors (Dompierre et al., 2007 J Neurosci 27:3571-83). Remarkably, the rate of transport of BDNF vesicles was reduced by overexpression of a non-acetylatable K40A (but not by wild type) α-tubulin (Dompierre et al., 2007 J Neurosci 27:3571-83). Thus, it appears that the effects of HDAC6 on vesicle transport are mediated by K40 deacetylation.

αTAT.

While two enzymes that deacetylate α-tubulin are known (HDAC6 and SIRT2), the identity of αTAT was for a long time unknown, despite the fact that the enzyme was first described 24 years ago (Greer et al., 1985 J Cell Biol 10:2081-2084). Partial purifications were reported, but without identification of the catalytic subunit (Maruta et al., 1986 J Cell Biol 103:571-579; Lloyd et al., 1994 Anal Biochem 216:42-46). Ohkawa and colleagues showed that ARD1-NAT1, a conserved N-acetyltransferase, affects neural differentiation, but it is not known whether this enzyme acts on α-tubulin and whether it can acetylate an internal K residue (Ohkawa et al., 2008 Genes Cells 13:1171-83). There was a suggestion that the conserved acetyltransferase, ELP3, acetylates K40 on α-tubulin, based on its ability to bind to microtubules, but functional data are not available (Gardiner et al., 2007 Traffic 8:1145-9). Recently, we used bioinformatics to identify a conserved protein, MEC-17, as a candidate for αTAT (see Examples I and II). Remarkably, MEC-17 mutations affect neuronal differentiation in C. elegans (Zhang et al., 2002 Nature 418:331-5; Way and Chalfie, 1989 Genes Dev 3:1823-33), and thus K40 acetylation could be important in neuronal development. We show that in Tetrahymena and zebrafish, MEC-17 is required for acetylation of K40 and that MEC-17 has a K40 αTAT activity in vitro (Examples I and II).

K40 α-Tubulin Acetylation in Touch Receptor Neurons in C. elegans.

In C. elegans, only one of 9 α-tubulin isotypes, MEC-12, has the K40 residue (Fukushige et al., 1999 J Cell Sci 112:395-403). MEC-12 mutations affect mechanosensation (mechanosensation defective-12), leading to a loss of touch sensation (Huang et al., 1995 Nature 378:292-295). Another mechanosensation gene, MEC-7, encodes aβ-tubulin (Savage et al., 1989 Genes Dev 3:870-881). The MEC-12 α-tubulin and the MEC-7 β-tubulin are expressed strongly only in the six touch receptor neurons (ALML, ALMR, AVM, PLML, PLMR, PVM) (Fukushige et al., 1999 J Cell Sci 112:395-403; Hamelin et al., 1992 EMBO J 11:2885-2893). Consistently, the anti-acetyl-K40 mAb, 6-11 B-1, revealed high levels of this PTM in the six touch receptor neurons (TRNs), and low levels in a few other neurons (Fukushige et al., 1999 J Cell Sci 112:395-403). The TRNs have microtubules composed of 15 protofilaments (pf), while other cell types in the worm have 11 pf microtubules (Chalfie and Thomson, 1982 J Cell Biol 93:15-23). Mutations of either MEC-7 or MEC-12 lead to loss of touch sensation and often result in the loss of 15 pf microtubules (Fukushige et al., 1999 J Cell Sci 112:395-403; Savage et al., 1989 Genes Dev 3:870-881). C. elegans provides an opportunity to address the function of acetyl-K40, because MEC-12 α-tubulin has restricted localization and its function is required for mechanosensation. The function of acetyl-K40 can be addressed directly by testing whether MEC-12 lacking an acetylatable K40 can supports touch sensation when introduced as a transgene into a mec-12 mutant. Fukushige and colleagues proposed to address this question by testing whether a MEC-12 α-tubulin with a K40Q mutation could rescue the touch sensation defect in mec-12 mutants; the MEC-12 K40Q transgene rescued the touch sensation defect (Fukushige et al., 1999 J Cell Sci 112:395-403). However, it is not possible to conclude from this result that K40 acetylation is not important because the K40Q mutation introduces an acetylation mimic: Q is an amino acid that structurally resembles an acetyl-K. Thus, the K40Q mutation does not test the requirement for K-acetylation. We propose a C. elegans MEC-12 model using a K40R MEC-12 mutation, as R is structurally similar to K but cannot be acetylated.

We found that in Tetrahymena and zebrafish a MEC-17 ortholog is required for the acetyl-K40 modification. MEC-17 was identified by Martin Chalfie's laboratory in C. elegans as a protein whose full activity is needed for the maintenance of TRN function (Zhang et al., 2002 Nature 418:331-5; Way and Chalfie, 1989 Genes Dev 3:1823-33). MEC-17 is restricted to TRNs and mec-17 is among ˜50 genes whose expression is dependent on MEC-3, a transcription factor that controls the TRN lineage (Zhang et al., 2002 Nature 418:331-5). Larvae homozygous for mec-17 mutation are touch-sensitive after hatching, but the sensation is partly lost in the L4 stage and absent in the adults (Way and Chalfie, 1989 Genes Dev 3:1823-33). While L1 larvae have functional TRNs, the TRNs experience a dramatic elongation of axons as the animal grows (Way and Chalfie, 1989 Genes Dev 3:1823-33). Thus, MEC-17 may be needed to support the axon elongation or maintenance of an already differentiated axon (possibly via K40 α-tubulin acetylation). Intriguingly, MEC-17 is needed to sustain the activity of the MEC-3 transcription factor (Way and Chalfie, 1989 Genes Dev 3:1823-33). An exciting possibility is that MEC-17, via K40 α-tubulin acetylation, participates in a feedback mechanism that links microtubules with gene expression and could play a role in maintaining the molecular identity of neurite projections. However, MEC-17 could have additional roles in early differentiation, because the only studied allele of MEC-17 may not be a null, and C. elegans has another MEC-17 paralog (see below).

ELP3 as a Regulator of α-Tubulin Acetylation.

Creppe et al. (2009 Cell 136:1-14) reported that E1p3 acetyltransferase is associated with an α-tubulin acetyltransferase activity in the mouse. A fraction enriched in E1p3 mediates weak acetylation of α-tubulin in vitro but prefers histone H3 as a substrate. E1p3 is also required for normal levels of K40 acetylation in vivo. Loss of E1p3 affects the differentiation of cortical neurons in the mouse and similar defects are caused by expression of a non-acetylatable K40A α-tubulin. Thus, the Creppe et al., provide another piece of evidence that α-tubulin acetylation is important in the nervous system, and identify E1p3 as a regulator of α-tubulin acetylation. It remains to be tested whether a recombinant E1p3 (expressed in bacteria) can act as a K40-specific αTAT. Note that we have predicted that another αTAT exists, distinct from MEC-17 (see Example II). Thus, E1p3 could be an αTAT, or an upstream regulator of MEC-17 or could activate another yet to be identified αTAT.

### K-40 Acetylation May Affect the Intrinsic Dynamics of Microtubules

Acetylation Assays with Soluble Tubulin and Microtubules.

MEC-17 is a marker of long-lived microtubules, and the levels of acetylation are higher on microtubules that are exposed to the enzyme for a longer time (Piperno et al., 1987 J Cell Biol 104:289-302; Webster and Borisy, 1989 J Cell Sci 92:57-65). Consistent with this, a partially purified aTAT activity preferred microtubules over unpolymerized tubulin as a substrate (Maruta et al., 1986 J Cell Biol 103:571-579). Thus, whether recombinant MEC-17 prefers microtubules over unpolymerized tubulin dimers in a reaction that includes the same concentration of tubulin that is either soluble or polymerized with taxotere (Wloga et al., 2008 Eukaryot Cell 7:1362-1372) can be tested. If this is the case, the enzyme may enter the microtubule lumen using polymer ends, and this can be tested by comparing the reactions rates on microtubules with different average length. Axonemes can be prepared and subjected to sharing by mild sonication. If shared microtubules support a higher rate of acetylation as compared to non-shared microtubules, this will indicate that the enzyme diffuses into the lumen using the ends of microtubules. Whether the enzyme preferentially acetylates sites that are closer to ends of axonemes can be tested using quantitative immunofluorescence with 6-11 B-1 anti-acetyl-K40 mAb.

Measuring the Effect of K40 Acetylation on the Dynamics of Microtubules In Vitro.

The relationship between acetyl-K40 and the dynamics of microtubules has been under scrutiny since the discovery of this PTM. Originally, the modification was found on axonemes of Chlamydomonas (L'Hernault and Rosenbaum, 1983 J Cell Biol 97:258-263; Greer et al., 1985 J Cell Biol 10:2081-2084). Axonemes have very stable microtubules that do not depolymerize under conditions that disrupt cytoplasmic microtubules (e.g., cold). Maruta and colleagues used a partially purified αTAT to acetylate microtubules in vitro and found that (in bulk) such microtubules polymerize and depolymerize at rates similar to microtubules not exposed to αTAT (Maruta et al., 1986 J Cell Biol 103:571-579). However, it appears that in vivo, acetyl-K40 has a stabilizing effect on microtubules (see Example II). These data do not necessarily contradict the in vitro observations made by Maruta and colleagues (Maruta et al., 1986 J Cell Biol 103:571-579), because the increased stability of acetylated microtubules could result from increased binding of stabilizing MAPs in vivo. However, it will be important to re-examine the potential effect of K40 acetylation on the intrinsic dynamics of microtubules in vitro. Soluble cytoplasmic tubulin from the MEC17-KO strain of Tetrahymena (Suprenant et al., 1985 Proc Natl Acad Sci USA 82:6908-6912) can be purified. The cytoplasmic tubulin of Tetrahymena has a low level of acetyl-K40 (Sharma et al., 2007 J Cell Biol 178:1065-79), and purifying it from a MEC 17-KO strain will ensure that the background of acetyl-K40 is extremely low.

MEC 17-KO tubulin can be polymerized with paclitaxel (Suprenant et al., 1985 Proc Natl Acad Sci USA 82:6908-6912), and the resulting microtubules can be used for in vitro acetylation with GST-MmMEC-17. Optimal assay conditions can be used to obtain the maximal level of K40 acetylation. The levels of acetyl group incorporation per tubulin dimer using a ³H-acetylCoA (Maruta et al., 1986 J Cell Biol 103:571-579) can be monitored. Control microtubules can be created that are non-acetylated but otherwise are treated in the same way and will be subjected to mock acetylation using a mutated acetyltransferase-inactive GST-MmMEC-17. The enzyme will be removed from microtubules using glutathione beads (after salt extraction if it binds to microtubules). The acetylated and non-acetylated microtubules will be subjected to depolymerization by cold and used for in vitro polymerization. Bulk rates of polymerization and depolymerization (in response to depolymerizing drugs, cold and tubulin dilution) will be measured. The levels of polymerized/soluble tubulin will be quantified using a sedimentation assay and western blotting with an anti-tubulin mAb. If an effect of acetyl-K40 in the above bulk assays is not detected, the approaches developed here will pave the way for more sensitive studies in the future, based on observations of single microtubules at the nanoscale (Gardner et al., 2008 Curr Opin Cell Biol 20:64-70). If this work indicates that acetyl-K40 acts in vivo by modulating MAPs or motors, the populations of uniformly acetylated or non-acetylated microtubules could be used for measurements of microtubule binding affinities of MAPs, and for single molecule assays on motors, to uncover the structural consequences of K40 acetylation.

### Expression of a Nonacetylatable (K40R) α-Tubulin May Phenocopy Mec-17 Loss of Function.

Express K40R MEC-12 α-Tubulin in a Mec-12 Mutant.

Whether the function of MEC-17 is primarily mediated by K40 α-tubulin acetylation can be tested. In such a situation, a mutation of K40 on α-tubulin that prevents its acetylation (K40R) should phenocopy the MEC-17 loss of function. In C. elegans, MEC-12 is the only K40 α-tubulin. MEC-12 transgenes (K40 or K40R) can be introduced into mec12(e1607) touch-defective animals (a likely null allele; Fukushige et al., 1999 J Cell Sci 112:395-403). To create transgenic strains, all transgenes can be introduced into a single site using a Mos1 transposon-based approach (Frokjaer-Jensen et al., 2008 Nat Genet 40:1375-83). The advantage of this approach is that the transgenes are expressed at levels similar to the endogenous gene (Frokjaer-Jensen et al., 2008 Nat Genet 40:1375-83). The touch responsiveness in rescued worms at the larval and adult stages can be quantified (Gu et al., 1996 Proc Natl Acad Sci USA 93:6577-82). If MEC-12 K40R animals have a mutant phenotype that resembles that of mec-17 mutants this will support our hypothesis that MEC-17 acts in vivo mainly by acetylating K40 on MEC-12.

Test Whether TBA-8 α-Tubulin is K-Acetylated.

Whether another K on α-tubulin is acetylated in the absence of K40 can be tested in C. elegans by immunoprecipitation of an in vivo tagged K40R MEC-12 α-tubulin and western blotting with a pan acetyl-K mAb. A more likely explanation of a lack of a touch sensation defect in worms expressing K40R MEC-12 is that MEC-17 acetylates both MEC-12 and another α-tubulin. MEC-12 is the only a-tubulin that has K40, and among the remaining 8 isotypes, all but one lack K40 or a K in the proximity of residue 40. The exception is the α-tubulin TBA-8, which has K41. The sequence of the H1-S2 loop of TBA-8 is divergent, but it is certainly possible that TBA-8 is also a substrate of K-acetylation by MEC-17, and TBA-8 alone could be sufficient to fulfill the function of α-tubulin acetylation. While the mec-12(e1607) worms lack a signal of acetyl-K40 using 6-11 B-1 mAb (Fukushige et al., 1999 J Cell Sci 112:395-403), this mAb likely would not recognize an acetyl-K41 on TBA-8 due to the divergence of the primary sequence. TBA-8 is expressed at the highest levels in a subset of neurons that also express MEC-7, the α-tubulin present in TRNs (Wormbase annotation). Commonly, tubulin isotypes that are co-expressed in the same cell type, copolymerize (Lopata et al., 1987 J Cell Biol 105:1707-1720). Thus, MEC-12 and TBA-8 could each form dimers with MEC-7 that then copolymerize to form microtubules inside TRNs and TBA-8 alone could provide a sufficient level of K-acetylation (but MEC-12 would still be required to fulfill other functions such as competence for assembly into 15 pf microtubules). If needed, whether TBA-8 is acetylated can be tested by expressing a transgene of TBA-8 with a FLAG tag, affinity purification and testing with a pan acetyl-K antibody on a western blot (as a positive control, MEC-12 can be tagged and purified). If a signal of acetyl-K is observed on TBA-8, the functional contribution of potential K41 acetylation of TBA-8, including its interactions with K40 acetylation of MEC-12 can be pursued. A study of the contribution of acetylation of TBA-8 will require a mutant allele, ideally a deletion so that the allele can be replaced by introducing a transgene that can not be acetylated. A TBA-8 deletion allele can be generated using a PCR-based screen of a deletion strain library.

Express K40R α-Tubulin in Zebrafish.

Whether the phenotype of MEC-17 morphants (hydrocephalus, small eyes, small body size, lack of startle response) can be phenocopied by injection of an mRNA encoding K40R α-tubulin (K40 mRNAs will be used as a control) can be tested. Zebrafish has several α-tubulin isotypes. The sequence of al tubulin, the major K40 α-tubulin in the nervous system, can be used (Goldman et al., 2001 Transgenic Res 10:21-33). One concern is that injecting α-tubulin mRNA could lead to side effects by causing a stoichiometric imbalance with its binding partner, α-tubulin. On the other hand, animal cells have an autoregulatory negative feedback mechanism that couples the rate of synthesis of α- and β-tubulin polypeptides to the levels of these proteins, by regulating the stability of their respective mRNAs (Ben-Ze'ev et al., 1979 Cell 17:319-325; Cleveland, 1989 Curr Opin Cell Biol 1:10-14). It appears that the two tubulin subunits are autoregulated independently of each other via specific mRNA degradations (Theodorakis and Cleveland, 1992 Mol Cell Biol 12:791-799). There is also evidence that excessive α-tubulin inhibits translation of α-tubulin mRNA (Gonzalez Garay and Cabral, 1995 Cell Motil Cytoskel 31:259-272). Thus, injection of α-tubulin mRNA into the zebrafish embryo could destabilize the levels of α-tubulin mRNA and the total levels of mRNA could be adjusted. The destabilizaton response could lead to a significant replacement of total α-tubulin mRNA with an ectopic variant. At any rate, control injections with a wild type α1 mRNA will determine whether overexpression phenotypes occur and the concentration of the injected mRNA will be adjusted accordingly. If overexpresion is a problem, mRNAs of other K40 α-tubulins (α6, and α8), can be used as these mRNAs could have a distinct stability or translation pattern. Alternatively, equal molar quantities of mRNAs encoding α- and β-tubulin, can be co-injected to balance the concentrations of tubulin subunits. If embryos injected with K40R α-tubulin mRNA (and not K40) show a phenotype that resembles the MEC-17 morphant phenotype, this will be consistent with a model that the major function of MEC-17 is to acetylate α-tubulin at K40. If the expression of a K40R α-tubulin leads to a loss of acetyl-K40, and yet the gross phenotype is normal, or distinct from the one observed in MEC-17 morphants, the role of MEC-17 may distinct from acetylation of K40 on α-tubulin and possibly based on acetylation of another protein.

Whether α-Tubulin with an Acetyl-Lysine Mimicking Glutamine (K40Q) Rescues the MEC-17 Loss of Function can be Tested.

C. elegans.

Fukushige and colleagues have determined that touch-insensitive mec-12(e1607) worms can be functionally rescued by a K40Q MEC-12 transgene, which suggests the acetylation mimic Q40 MEC-12 is functional. Whether a K40Q MEC-12 transgene rescues the touch sensation defect in mec-17 mutant worms can be tested. Wild type K40 and K40R non-acetylatable MEC-12 transgenes can be used as controls. If K40Q MEC-12 restores the touch sensation in the mec-17 mutant, and the control transgenes do not, this would indicate that the K40Q acetyl-lysine mimicking mutation bypasses the need for MEC-17, which would imply that the main function of MEC-17 is to acetylate K40 on α-tubulin.

If K40Q α-tubulin does not rescue the touch sensation defect of a mec-17 mutant, the K40Q MEC-12 transgenic protein may not be sufficiently represented in the microtubule copolymer with the endogenous MEC-12 and possibly with TBA-8. To address this possibility, rescue experiments can be performed using worms that have a mec-17 mutation and a likely null allele mec-12(e1607) (Fukushige et al., 1999 J Cell Sci 112:395-403). If a rescue of mec12(e1607); mec-17 double animals by K40Q MEC-12 is not observed, it may be possible that such a rescue requires replacement of both MEC-12 and TBA-8 with an a-tubulin that encodes an acetyl-K mimicking Q. Whether TBA-8 is K-acetylated can also be tested. The function of MEC-17 can be bypassed by introducing multiple copies of K40Q MEC-12, or a combination of K40Q MEC-12 and K41Q TBA-8 transgenes, to increase the contribution of acetyl-K mimicking α-tubulin relative to the endogenous TBA-8 in microtubules in TRNs.

Zebrafish.

In an analogous fashion, a rescue of the MEC-17 morphant zebrafish by introducing an mRNA for K40Q α-tubulin can be attempted. The technical aspects have been discussed above. The extent of rescue will be assessed in living embryos by a restoration of normal embryo morphology and a normal startle response. A rescue of the morphant phenotype would indicate that the in vivo role of MEC-17 is in acetylating α-tubulin at K40. A lack of rescue would indicate that either MEC-17 functions in a distinct pathway (e.g. by acetylating another protein), or that the transgene protein has not reached the proper level of incorporation into microtubules. mRNAs that encode K40, and K40Q α-tubulins with an epitope tag can be injected and the levels of transgenic proteins and acetyl-K40 can be evaluated. If needed, attempts can be made to increase the content of transgenic K40Q a-tubulin by increasing the amounts of injected mRNA or by using K40Q variant mRNAs of other α-tubulin isotypes.

### Whether Inhibition of HDAC6 α-Tubulin Deacetylase Rescues a MEC-17 Deficiency can be Tested.

HDAC6 is a conserved deacetylase for K40 on α-tubulin. An inhibitor, tubacin, is available that is specific to HDAC6 (and not to other HDAC enzymes that modify histones) (Haggarty et al., 2003 Proc Natl Acad Sci USA 100:4389-4394). Tubacin inhibits HDAC6 in species ranging from Volvox to mammals (Haggarty et al., 2003 Proc Natl Acad Sci USA 100:4389-4394; Cheng et al., 2006 J Phycol 42:417-422), and thus it is expected to be effective against the C. elegans (HDA-6) and zebrafish (ZDB-GENE-030131-3232) orthologs.

C. elegans.

The mec-17(u236) allele may not be null (Chalfie and Driscoll, “The saga of cloning MEC-17continues . . . ” Meeting Abstract. East Coast Worm Meeting New Brunswick, N.J.; June, 1996), and thus removing an opposing activity of HAD-6 in this background could rescue the deficiency of MEC-17. Even if all available MEC-17 alleles are null, it is possible that the second DUF738 protein, W06B11.1, contributes to K40 a-tubulin acetylation, and thus the net acetyl-K40 levels (and its function) could be enhanced by inhibition of HDAC6 in mec-17 mutant worms. Thus, whether tubacin can partially or completely rescue the phenotype of the mec-17 mutants of C. elegans can be determined.

Zebrafish.

An analogous experiment will be performed in zebrafish, by incubation of MEC-17 morphants with tubacin (or niltubacin). To ensure a partial reduction of MEC-17 function, the amount of MOs injected can be reduced. If tubacin has no detectable effect, whether the treatment increases the levels of acetyl-K40 can be tested using western blots and quantitative immunofluorescence. If there is no increase in acetyl-K40, this could be caused by insufficient drug uptake. As an alternative strategy, HDAC6 MOs (and scrambled MOs as a control) can be co-injected with the MEC-17 MOs at the 1-4 cell stage. At least 200 embryos will be examined in each class (Hagos and Dougan, 2007 BMC Dev Bio 7:22; Fan et al., 2007 Dev Biol 310:363-78). If we find that embryos co-injected with MEC-17 MO and HDAC6 MO have a less severe phenotype than those co-injected with the MEC-17-MO and control MOs it is likely that HDAC6 acts antagonistically to MEC-17.

MEC-17 may acetylate another substrate, which is important during neuronal differentiation. Zebrafish can be used in an attempt to identify additional substrates of acetylation by MEC-17. Zebrafish can be used rather then C. elegans, because of the availability of a large quantity of tissue for biochemical studies and high concentration of neural cell types within the developing embryo. Immunoprecipitation with pan-acetyl-K antibodies can be used to identify acetylated proteins that are less abundant in MEC-17 morphant embryos as compared to wild type, and such proteins can be identified by standard approaches of mass spectrometry. Alternatively, embryos can be labeled with radioactive acetate (in the presence of cycloheximide) and use 1D SDS-PAGE and fluorography to identify proteins whose levels of acetylation decrease in morphants as compared to controls. Candidate novel substrate proteins can be studied functionally in both C. elegans and zebrafish. In vitro assays can be used to determine whether such proteins can be acetylated by MEC-17.

### Whether MEC-17 Affects Neuronal Differentiation and Neuronal Microtubules In Vivo can be Tested.

In C. elegans, a subset of neurons fails to function properly without MEC-17. The phenotype of MEC-17 morphant fish is also consistent with defects in the nervous system. The emphasis on microtubules is justified in the light of growing evidence for role for microtubules in the differentiation of neurons (Witte and Bradke, 2008 Curr Opin Neurobiol 18:479-87). In C. elegans a focus will be on the organization and dynamics of microtubules. C. elegans offers a specific advantage in studies at the ultrastructural level. The presence of large diameter microtubules will facilitate the identification of specific TRNs and will help in 3D reconstructions of microtubule bundles. Zebrafish will be used primarily for observations on live neurons during differentiation. The zebrafish model offers advantages for time lapse imaging studies that will be focused on the well characterized primary motor neurons.

MEC-17 May Affect the Morphological Differentiation of TRNs in C. elegans.

It is not known to what extent TRNs can differentiate without MEC-17 function. To characterize the MEC-17 deficiency phenotypes, animals expressing a tagged protein that marks TRNs in either wild type or mec-17 mutant background can be created. A preference is to express a tagged version of MEC-12, to simultaneously follow the TRNs and the gross organization of microtubules. To this end, a rescue of mec-12(e1607) mutant with a transgene that encodes MEC-12::GFP under control of its own promoter can be attempted. Based on the work in C. elegans, it is likely that addition of GFP to α-tubulin will be tolerated (Pelletier et al., 2004 Curr Biol 14:863-73). Alternatively, MEC-7, a TRN-specific α-tubulin with GFP, can be tagged and used to rescue a mec-7 mutant (Savage et al., 1989 Genes Dev 3:870-881; Hamelin et al., 1992 EMBO J 11:2885-2893). Alternatively, the TRN cytoplasm can be marked with a GFP expressed under a TRN-specific promoter (Pmec-4::gfp) (Royal et al., 2005 J Biol Chem 280:41976-86; Bianchi et al., 2004 Nature Neurosci 7:1337-44). By live observations based on GFP, how the mec-17 alleles affect the morphological differentiation of TRNs can be determined. In wild-type animals, the TRNs are functional in the L1 stage, but the axon processes undergo extensive growth during subsequent larval stages, which correlates with the growth of the animal. For example, the axon of ALM grows in size by a factor of 4 between the L1 and L4 stages (Wu et al., 2007 Proc Natl Acad Sci USA 104:15132-7). Thus, a defect in the differentiation or growth of TRNs may be detected (e.g. incorrect number, orientation, or elongation of axons).

An important question is whether the late onset of the touch sensation defect that was reported for the single characterized mec-17 allele, mec-17(u265), reflects a specific function of MEC-17 in late differentiation events (e.g. in axon elongation or in maintenance of a differentiated neuron), or results from an incomplete loss of function of the mec-17(u265) allele. Specifically, a simple explanation of the mec-17(u265) phenotype is that the residual activity of MEC-17(u265) is sufficient to support the small TRNs of early larvae, but is insufficient for the fully elongated TRNs in adults. If the mec-17(ok2109) animals display defects in the early larvae, for example in the differentiation of neurons, this will support a wider role for MEC-17 that is not restricted to the TRN elongation and maintenance stages. If MEC-17 functions specifically in the late stage of TRN differentiation, animals carrying the null allele, mec-17(ok2109), will also show a late onset defect. If results are consistent with a late onset function for MEC-17, this will be very exciting because such a result would indicate that acetylation of K40 is required for the maintenance of the differentiated state in neurons. For example, the presence of the PTM on axonal microtubules, by a feedback mechanism, could signal back to the cell body and contribute to the maintenance of axonal identity. This model is supported by the observation that MEC-17 activity is needed for the continuing expression of MEC-3, the transcription factor that controls the TRN lineages (Way and Chalfie, 1989 Genes Dev 3:1823-33). Note that our data in zebrafish are consistent with this model, because axons of motor neurons appear to form despite strong reduction in K40 acetylation (Example II and FIG. 8).

MEC-17 Deficiency May Affect the Organization and Dynamics of Microtubules in C. elegans.

It will be of important to determine how a deficiency in MEC-17 affects microtubules. The axonal processes of TRNs are filled with unique wide-diameter microtubules that are composed of 15 pf. While not proven directly, it appears that the 15 pf microtubules are required for touch sensation. For example, mutations of MEC tubulins lead to a loss of touch sensation that is often associated with loss of 15 pf microtubules, while the 11 pf microtubules are still present (Fukushige et al., 1999 J Cell Sci 112:395-403; Savage et al., 1989 Genes Dev 3:870-881). The 15 pf microtubules stain intensely with tannic acid in TEM. Chalfie et al. have reconstructed the organization of microtubules in TRNs axons and concluded that they are filled with a large number of relatively short and partly overlapping 15 pf microtubules (Chalfie and Thomson, 1982 J Cell Biol 93:15-23; Chalfie, 1982 Cold Spring Harbor Symposia on Quant Biol 46(Pt 1):255-61).

Transmission Electron Microscopy in C. elegans.

The organization of microtubules in 3D, in sections of wild type and mec-17 mutants can be reconstructed. The choice of the most informative allele composition for MEC-17 and W06B11.1 and stage in development (L1-L4 or adult) will be determined based on examination of the gross organization of microtubules by confocal microscopy and mutant phenotype kinetics (as described above). Animals with an allele combination, and at a stage, in which the touch sensation phenotype is already strongly manifested will be chosed. Later stages will be avoided to reduce the chance of studying secondary effects. For optimal preservation, high pressure freezing with freeze-substitution can be used, as applied to C. elegans by Evans and colleagues, and serial sections for segments of a total of 1-2 μm can be obtained (Evans et al., 2006 J Cell Biol 172:663-9). Serial images will be aligned in 3D (Mastronarde et al., 1992 J Cell Biol 118:1145-1162; Kremer et al., 1996 J Struct Bio 116:71-6). How MEC-17 deficiency affects the diameter (pf number), length, and spacing of microtubules in TRN axons can be determined. A study of animals with a K40R MEC-12 mutant α-tubulin can be included. As some observations suggest that acetyl-K40 promotes microtubule stability, TEM could reveal that the loss of MEC-17 leads to a reduction in the number or length of microtubules, which could in turn affect either the mechanical functions of 15 pf microtubules (in the touch signal transduction), or the motor-based transport.

Tubulin PTM Markers as Reporters of Microtubule Dynamics.

The dynamics of microtubules in the MEC-17 mutants by quantifying the content of newly added tubulin can be evaluated. In most eukaryotes, α-tubulin that is incorporated into microtubules is subjected to detyrosination (removal of the C-terminal Y), sometime after assembly (Barra et al., 1982 J Neurochem 38:112-5; Gundersen et al., 1987 J Cell Biol 105:251-264). The Nna1 carboxypeptidase is suspected to be responsible for detyrosination (Kalinina et al., 2007 Faseb J 21:836-50; Rodriguez de la Vega et al., 2007 Faseb J 21:851-65) and C. elegans has a single Nna1 gene, F56H1.5. The more dynamic the microtubule is, the higher the content of tyrosinated α-tubulin. The tyrosinated form can be detected in C. elegans using the YL1/2 mAb (Stear and Roth, 2002 Genes Dev 16:1498-508). In mammalian neurons, the ratio of tyrosinated/total tubulin decreases as a function of differentiation and increasing stability of axonal microtubules (Witte et al., 2008 J Cell Biol 180:619-32). Using quantitative confocal immunofluorescence, the ratio of tyrosinated/total tubulin is altered in TRNs of mec-17 can be determined. An increase in the relative content of tyrosinated tubulin would indicate that the mutant microtubules fail to undergo stabilization during differentiation of TRN.

How MEC-17 affects another marker of microtubule maturation, polyglutamylated tubulin, can be evaluated. Glutamylation is a polymeric modification, based on side chain peptides composed of a variable number of Es added to primary sequence Es (Eddé et al., 1990 Science 247:83-85). In mammals, glutamylated tubulin accumulates on neuronal microtubules as the nervous system develops (Audebert et al., 1994 J Cell Sci 107:2313-2322). As part of the maturation of neurons, the side chains undergo elongation (Audebert et al., 1994 J Cell Sci 107:2313-2322). A polyclonal antibody (polyE) is available that specifically recognizes long side chains that was developed by the Gorovsky lab (Shang et al., 2002 J Cell Biol 158:1195-1206) and was recently characterized (Wloga et al., 2008 Eukaryot Cell 7:1362-1372). A decreased ratio of polyglutamylation/total tubulin will indicate that MEC-17-deficient neurons are defective in their maturation, and possibly in stabilization of microtubules.

Microtubule Drugs as Probes of Microtubule Dynamics.

A change in the levels of a PTM marker that preferentially labels either old or dynamic microtubules could reflect a change in the turnover rate of microtubules, but could also be caused by other processes such as steric interactions among different tubulin PTMs or effects of MAPs that could affect the binding of PTM enzymes. Thus, drugs can be used as a complementary approach to probe the dynamics of TRN microtubules. Microtubule-depolymerizing compounds (benomyl, nocodazole, and colchicine) depolymerize microtubules in C. elegans (Chalfie and Thomson, 1982 J Cell Biol 93:15-23). Interestingly, colchicine causes depolymerization of microtubules specifically in the TRNs (where it affects both 15 and 11 pf microtubules) (Chalfie and Thomson, 1982 J Cell Biol 93:15-23). Worms treated with colchicine loose touch sensation, but lack other defects such as uncoordinated movement, that were observed with other depolymerizing drugs (benomyl, nocodazole) (Chalfie and Thomson, 1982 J Cell Biol 93:15-23). Thus, colchicine could a have higher affinity for tubulin in TRNs (e.g. MEC-7/MEC-12 dimers). Using anti-tubulin immunofluorescence, whether mec-17 mutations affect the sensitivity of TRN microtubules to colchicine and to a microtubule-stabilizing compound, paclitaxel, can be determined. If microtubules in TRNs of mec-17 mutants are more labile, this could be manifested in an increase in sensitivity of TRN microtubules to colchicine (e.g. depolymerization at a lower drug dose or in a shorter time) and increased resistance to paclitaxel-induced hyper-polymerization. Whether the touch sensation function is affected by the microtubule drugs can also be tested. Wild type and mec-17 mutants can be grown on agar plates containing either colchicine or paclitaxel and test for touch sensation at distinct stages of development. If MEC-17 acts primarily by promoting the stability of microtubules, mec-17 mutants (at early larval stages) could loose touch sensation at lower concentrations of colchicine as compared to wild type. Moreover, the mec-17 mutant worms could be more resistant to paclitaxel (as we observed for MEC 17-KO Tetrahymena, Example II and FIG. 3i), and paclitaxel could rescue the touch sensation defect in MEC-17 mutants. A rescue by paclitaxel would support the model that MEC-17 acts in vivo (via K40 acetylation) by stabilizing microtubules.

How MEC-17 Affects Microtubule-Interacting Proteins in C. elegans can be Determined.

Tau MAP Homolog, PTL-1.

In mammalian cells, Tau is restricted to the axon, while MAP2 is mainly located in the cell body and dendrites. These two proteins belong to a family of so-called “structural MAPs”, that decorate the outside surface of microtubules, control the spacing between microtubules, and make microtubules more stable (Dehmelt and Halpain, 2005 Genome Biol 6:204). As discussed, the stabilizing effect of MEC-17-mediated K40-acetylation in vivo could be mediated by structural MAPs. Specifically, MAPs could bind to acetyl-K40 microtubules with higher affinity. It thus will be important to test whether the levels of structural MAPs and are affected in the mec-17 mutants. C. elegans has a single gene that encodes a Tau-like protein, PTL-1, that is expressed only in a few cell types: 1) embryonic epidermis; 2) head and ventral cord neurons; and strikingly in 3) all but one TRNs (ALML, ALMR, AVM, PLML, PLMR) (Goedert et al., 1996 J Cell Sci 109:2661-72). The only TRN that is negative for PTL-1 is PVM (Goedert et al., 1996 J Cell Sci 109:2661-72), which also is the only neuron that is not essential for touch sensation, based on laser ablation studies (Chalfie and Sulston, 1981 Dev Biol 82:358-70). Importantly, the ptl-1 mutant animals have reduced touch responsiveness and a ptl-1 mutation acts as a dominant enhancer of mec-7 (Gordon et al., 2008 Dev Genes Evol 218:541-51). Quantitative immunofluorescence can be used to determine how MEC-17 mutations affect the ratio of PTL-1/tubulin with anti-PTL-1 antibodies. A decrease in the PTL-1 levels would suggest that MEC-17, via K40 acetylation of α-tubulin, stimulates the binding or retention of PTL-1 and possibly other yet to be identified stabilizing MAPs to microtubules. In vitro acetylation assays can be used to evaluate the role of K40 acetylation in Tau binding. These studies could be relevant to the pathology of AD, based on the role of human Tau in AD (Sahara et al., 2008 Curr Alzheimer Res 5:591-8) and the fact that reduced stability of microtubules is one of the phenotypes of AD (Butler et al., 2007 Eur J Pharm 562:20-7).

Motor Proteins in TRNs.

The potential effects of MEC-17 on motor proteins can be addressed. In a breakthrough study, Reed and colleagues showed that in vitro, kinesin-1 binds more strongly to, and moves faster on microtubules that have acetyl-K40 (as compared to R40) (Reed et al., 2006 Curr Biol 16:2166-2172). In an unpolarized neuron, ectopic kinesin-1 preferentially accumulates in a single neurite before it elongates into an axon (Jacobson et al., 2006 Neuron 49:797-804). Moreover, among the multiple neurites in an unpolarized neuron, there is usually a single neurite that has elevated levels of acetyl-K40 (Witte and Bradke, 2008 Curr Opin Neurobiol 18:479-87) (presumably this is the same neurite that accumulates kinesin-1). It is tempting to speculate that acetylated microtubules stimulate a selective motor-driven transport into and within the axon. The increased stability of axonal microtubules (Witte et al., 2008 J Cell Biol 180:619-32) could also be a downstream consequence of increased trafficking by axonal motors that could deliver cargo in the form of stabilizing MAPs (Ohba et al., 1993 Biochim Biophys Acta 1158:323-332). Kinesin UNC-104 is a motor that in a major way contributes to synaptic trafficking in C. elegans (Hall and Hedgecock, 1991 Cell 65:837-847), and is expressed in the TRNs (Zhou et al., 2001 J Neurosci 21:3749-55). Importantly, GFP::UNC-104 transgene rescues the unc-104 mutant and could be used for live observations of motor motility (Zhou et al., 2001 J Neurosci 21:3749-55). GFP::UNC-104 can be expressed in both wild type or mec-17 mutant animals. Following the methodology established by Zhou and colleagues (Zhou et al., 2001 J Neurosci 21:3749-55), time-lapse confocal microscopy can be used to determine whether a deficiency in MEC-17 affects the parameters of motor trafficking (such as the frequency, rate, and run length of the moving motors) specifically in TRNs. One concern is that the lack of activity of MEC-17 could lead to loss of microtubule tracks, and any effects on motors would be secondary. It is unlikely based on the fact that motor neurons in zebrafish embryos grow axons when MEC-17 is severely depleted (Example II and FIG. 8). A systematic study of larvae in various stages could reveal defects in motor transport that occur prior to, or coincide with, the onset of touch deficiencies.

### Consequences of Loss of Function of MEC-17 in Zebrafish

While C. elegans offers an excellent genetic model and a remarkable level of simplicity for studies on MEC-17, there is a need to perform functional studies in another model as well. One justification for this is that the C. elegans may be unusual because TRNs have unique large diameter microtubules that could have specialized roles in touch sensation. Also, a complication to studies in C. elegans is that this species has two DUF738 domain proteins. Finally, zebrafish offers established methodology for time lapse studies of single neurons in live animals. Thus, how a deficiency in MEC-17 affects neurons in zebrafish can be investigated. A subset of neurons are well characterized and are affected by MEC-17 depletion, the primary motor neurons (PMNs) in the trunk. Each trunk segment has 3 PMNs: caudal (CaP), middle (MiP), and rostral (RoP) (Myers et al., 1986 J Neurosci 6:2278-89; Eisen et al., 1986 Nature 320:269-71). PMNs are the first neurons to exit the cell cycle and differentiate. Each of the 3 PMNs can be identified by the position of its cell body along the anterior-posterior axis within the spinal chord, the trajectory of the axonal projection, and the region of the muscle it innervates (Myers et al., 1986 J Neurosci 6:2278-89; Westerfield et al., 1986 J Neurosci 6:2267-77). Most of the work will focus on the well characterized CaP that sends a prominent axon to the ventral axial muscle.

Live Imaging of PMNs.

We will perform observation on live embryos to determine whether MEC-17 plays a role in the growth and pathfinding of PMNs axons. To follow the CaP axon, MEC-17 or control (scrambled) MOs can be injected into the Tg(HB9:GFP) embryos that express GFP in both the cell body and axons of PMNs (Flanagan-Steet et al., 2005 Development 132:4471-81). Live embryos will be anesthetized, embedded in low melt agarose and recorded by time-lapse photography under a confocal microscope at a rate of 1 image every 5 minutes from 17 hpf to 20 hpf (Flanagan-Steet et al., 2005 Development 132:4471-81). The rate of axon growth and branching frequency of the growth cone can be monitored to determine the frequency of pathfinding errors.

Imaging studies can be performed on single neurons. The Tg(HB9-GFP) embryos can be injected with MEC-17 or control MOs. At the blastula stage, small groups of cells from the MEC-17 or control MO embryos can be transplanted into untreated embryos. In these experiments, the transgenes themselves will serve as a lineage tracer to mark the transplanted cells. If any descendants of the injected cells become motor neurons, they will express GFP and we will observe them using time-lapse microscopy. We expect only one or few cells per embryo will become motor neurons. Thus, the experiment will be useful in two ways. By imaging single cells recording paths of multiple PMN axons that cross each other can be avoided. Second, if an effect specific to MEC-17 MO-treated cells inside an otherwise untreated embryo is observed, this will indicate that the function of MEC-17 occurs autonomously within the neuron, which would be in agreement with the already documented cell type-specific effects of MEC-17 in C. elegans (Way and Chalfie, 1989 Genes Dev 3:1823-33).

Evaluation of Synapses, Kinesin-1, and Tau.

The pattern of synaptotagmin-2, a marker of nerve terminals using the Znp-1 mAb can be investigated (Fox and Sanes, 2007 J Comp Neurol 503:280-96; Trevarrow et al., 1990 Neuron 4:669-79). The formation of a functional synapse at the neuromuscular junction is required to maintain clustering of acetylcholine receptors (AchR) in the post-synaptic cell. Thus, live Tg(HB9:GFP) embryos injected with MEC-17 MOs and incubated with Texas Red-α-bungarotoxin to detect AchRs at 17-24 hpf and 48 hpf, can be examined using confocal microscopy. Fixed embryos can also be examined at these stages using the anti-AchR mAb and the F59 mAb, which labels muscle fibers (Miller et al., 1985 J Cell Biol 101:643-50). A diffuse staining pattern along the muscle fiber would indicate that the synapse is non-functional. If defects in the distribution of synaptic or post-synaptic markers are observed, this will indicate a defect in the axonal transport.

One explanation for potential defects in the synapse organization is that MEC-17 activity (via acetylation of microtubules) is required for kinesin-1 activity, a major motor that moves synaptic vesicles (Vale et al., 1985 Cell 42:39-50) and whose motor domain prefers K40-acetylated microtubules in vitro (Reed et al., 2006 Curr Biol 16:2166-2172). Zebrafish kinesin-1-GFP (Kif5ba) can be expressed exclusively in the primary motor neurons, by injecting a plasmid encoding kinesin-1-GFP under control of the HB9 promoter into embryos. If there are difficulties in detecting the signal in PMNs, we will express GFP-tagged versions of two closely related kinesin-1 motors that zebrafish expresses (KIF5a, Kif5bb). Alternatively, JIP-1 protein, a kinesin-1 cargo that moves into axons (Matsuda et al., 2003 J Biol Chem 278:38601-6) can be tagged. If a depletion or mislocalization of kinesin-1 or JIP-1 in the MEC-17 morphants is observed, this will indicate that the endogenous kinesin motors do not interact efficiently with the axonal hypo-acetylated microtubules. The levels and patterns of the microtubule-associated protein, Tau, the binding of which could be facilitated by K40-acetylation can also be evaluated. Antibodies against zebrafish Tau protein have been made (Tomasiewicz and Wood, 1999 Cell Motil Cytoskel 44:155-67).

It would be of high interest if these observations establish that the axon initially develops normally, but is affected at a later time during development, which would be indicated by a late onset drop in the content of Tau, kinesin-1 or its cargo, or a decrease in the presence of synaptic markers. Such results would be consistent with a model that MEC-17, via K-40 acetylation, is needed for the maintenance of axonal identity of the cell projection in an already differentiated neuron.

Immunolocalization of MEC-17.

To explore how MEC-17 is spatially regulated, immunofluorescence can be used. Commercially-available antibodies against mammalian MEC-17 (Abcam) can be used, or if needed, polyclonal antibodies that recognize the zebrafish protein (using an outside contractor) can be made. It is well established that K40 acetylation is highly enriched in axons of nerves in zebrafish, to the extent that some authors describe the 6-11 B-1 mAb as a “pan-axonal marker” (Devine and Key, 2003 Methods Cell Sci 25:33-7). If MEC-17 is enriched in axons of diverse neurons (as compared to the cell body), in a pattern that corresponds to acetyl-K40, this will be consistent with the spatial regulation of MEC-17 localization being a major mechanism that establishes the pattern of K40-acetylated microtubules.

These studies use complementary approaches in C. elegans and zebrafish, to uncover the consequences of MEC-17 loss of function, in the course of neuronal differentiation and maintenance. The effect of mEC-17 on the morphology of neurons, organization of microtubules, and localizations of Tau MAPs and kinesin motors can be evaluated. Thus, these studies can provide clues as to what are the major consequences of loss of MEC-17 activity in vivo and can identify likely effectors of K40 acetylation. Whether the effects of loss of MEC-17 activity on microtubule effectors (such as motors or Tau) are mediated by lack of proper K40 acetylation of α-tubulin, using in vitro assays (e.g. microtubule binding or motor motility assays) may also be addressed.

In analogy to the “histone code” (Strahl and Allis, 2000 Nature 403:41-5), there could exist a “tubulin code” that marks microtubules on an organelle-wide scale (Verhey and Gaertig, 2007 Cell Cycle 6:2152-60). Whether the “tubulin code” is important, specifically in neural development, can be tested. The function of this acetylation of K40 can be tested in two ways: by inactivating the modification enzyme, and by mutating the modification site on tubulin.

C. elegans and zebrafish can be used as two models for functional studies. The use of two distinct models for functional studies offers distinct advantages. The use of two models with distinct methodologies will provide an opportunity to use alternative strategies to address the same question. Two very different methodologies available for C. elegans and zebrafish can be used to link the function of MEC-17 to α-tubulin acetylation in vivo. The specific advantages of each model can also be used to study the loss of function phenotypes for MEC-17. Specifically, in C. elegans, the presence of large diameter microtubules in TRNs, to facilitate identification of specific TRNs in ultrastructural studies and to aid reconstruction of 3D organization of the microtubular cytoskeleton in wild type and mec-17 mutant worms is advantageous. In zebrafish, the ability to observe single neurons during differentiation and specifically, the process of axonal growth and pathfinding in live animals is advantageous.

The power of C. elegans genetics to identify interactors of MEC-17 and acetyl-K40 can also be utilized. For example, screens for suppressors and enhancers of the touch sensation defect in mec-17 mutants should be feasible following the methodology established by Martin Chalfie's laboratory.

### Example IV

MEC-17 and MEC-17 homologs can be viewed at a number of databases including the National Center for Biotechnology Information (NCBI) website, available on the World Wide Web at www.ncbi.nlm.nih.gov/, or the Miami University BioInfoLAB, available on the World Wide Web at http://www.bioinfolab.org/bioinfolab3p1/tiki-index.php. MEC-17 and MEC-17 homologs can be identified by any reference number corresponding to the database. For example, sequences found in the NCBI database may be identified by either an NCBI gi number or an NCBI accession number. Non-limiting examples of MEC-17 and MEC-17 homolog sequences include the following.

Ce, (mec-17) Caenorhabditis elegans gi|17540784|ref|NP_501337.1|
SEQ ID NO: 41
MQVDADLRPILGPQLVRLDPMRVKQLQDPIVYEAIDNLAKLSAHCLQLRTPLTTCEKLINSDSTLYLSWKYDEE
EKVSRLMGFAKVGRKKLFLYDSQMQTYEGEILCLLDFYVHFSCQRQGVGQQILDYMFSQEHTEPYQLALDNPSV
TLLGFMSQKYGLIKPVWQNTNFVVFEELFLALSAENGIEKPPPDGWRRPMTPRRLGTGMTDTRWLQHAVSGHQS
KGNAMAAPVDADMTPQGALSNRAHQAKARKAHILSSKPLW
Ce, W06B11.1 Caenorhabditis elegans gi|17570273|ref|NP_508981.1|
SEQ ID NO: 42

MEIAFDLSTIFTDNIQRLTRTDLLKYGPKRYWAVAQSIDCLGEMSSKFHGWKRVITMYDKIVDHDEEQTTYIMW
EKVNGSKSILKGLLRVGYKTLYLTDNEQNQYMEKAMCILDFFVVPTEQRSGNGFKMFDEMLKAENVTVDQCAFD
KPSAALQQFLEKYYDRKDLVWQSNKYALCSNFFIGRHPTVPFTPRQTKRASRASSAVSSHASSRNTSPIGRNRP
RHDSVADLMRQDMLAGVRAEVDPNSPTGLKNARDFGHRRIW
Ci, Ciona intestinalis gi|198422961|ref|XP_002123997.1|

SEQ ID NO: 43
MEFDFNINHLFPDKITLVGENSSYRKHANSKILQRNLQIVIEVLGQRSARAQQLTGSITTLLKSQLNNQRIYVL
KEANANNGLGCVIGFLKTGKKRLFVLDRDGNHNEMNPLCVLDFYVHESQQRKGCGLCLFKHMLHVEGVKASHLA
IDRPSHKFISFLKKHFSLWATVPQVNNFVIFDGFFKNRSDTVRNNNAKSEWNNRQFRPLSRAVSDDIIKRNRFS
HDVPNKVNGLPPLPPNRRDRYGDNDVVTNPYRRNSTNQNGHVKGDVLQGSHMADKKDIGEERAKDNALPGIATN
QLEESRVQTNPQEVTENAYNGMNKHSSPVDQLKCNSLSQDQKSSPKIARKPATPPHLKPSSIIDELKAKDAAYL
SNRNLSTQNQSRLWYQRQLQGTRSSWNVLGVPTRPFWNAQ
Dm, (CG3967) Drosophila melanogaster gi|21355363|ref|NP_648310.1|
SEQ ID NO: 44
MVEFRFDIKPLFAQPIIKVTSNLLPNTFRGDRRQCLDATSKMTEIIDQLGQLSATSQGLSKPVTTAQRLRMSDN
QTIYLLADNEAGHNGAVLGLLKVGTKNLYLFDEAGKTRMVEQTPSILDFYVHESRQRAGLGKRLFQTMLNEEQW
TARKCSVDRPSEKLLSFLSKHYGLKRIIPQANNFVLYEGFFNDGESGNGGGNGHANGTPNGLHITNSPNTHLFG
ATYLGEDSNQRRGSQQQTTPNARLQQITQISPSGRYGAKRPTCSMAEIIHAGNSKGGNGNGSAEANSGNGNHDI
PEIAEQLQRQSLADLEANSYEPEPEVEPEPEPEPEPEPEPEVITPPSPPPKSHTPTPPSVRSPEVAESIVGDNR
RAPAFQLSKQHTGMKNRSFGVGMAVMPSSKMEFDQMEREDFGVVKINRPPGHEVTSPGQDNTDAMSTVSSGGGG
LTDQGYYDLKFYHNKLW
Dm, (CG17003) Drosophila melanogaster gi|20129077|ref|NP_608365.1|CG17003
SEQ ID NO: 45
MVEFAFDIKHLFPQSIIRVQAHSLRPKVTQCRRYAQTERGKSTMTSCRLSEILNIMGKLSADAQGLCHAVTSAD
KLASDQVVYLMADKAAGHWEITGLLKVGTKDLFVFDQGGCYRRLNQTPAILDFYVHESRQRCGQGKLLFEWMLE
KQGWSAHKCTVDRPSNKMLAFMAKHYGLVRTIPQGNNFVLYEGFFDDPITTCKSASGLQATGSGCRSRSQGHYV
RQEQDQAQIKHGQANRNTVQNDANSGPFRQDQKIVVGTSIYRRRWKSPRTLARAGCREVSGGRRF
Dr, Danio rerio gi|34784851|gb|AAH56749.1|Zgc: 65893
SEQ ID NO: 46
MDFPYDLNALFPERISVLDSNLSAGRKAHGRPDPLPQVTTVIDELGKASSKAQQLPAPITSAAKLQANRHHLYL
LKDGEQNGGRGVIVGFLKVGYKKLFLLDQRGAHLETEPLCVLDFYVTETLQRHGYGSELFDFMLKHKQVEPAQM
AYDRPSPKFLSFLEKRYDLRNSVPQVNNFVVFAGFFQSRSAVQLRKVPPRKPEGEIKPYSLMEREVVREEQRVL
PWPFVRPGGPPHSPPLLPSSPQSRSLSVGSSPSRAPLRPAAATVLQQGQTPSSPLNDSCRAKRTSSLNRSRLSF
H
Gl, Giardia lamblia gi|159112038|ref|XP_001706249.1|
SEQ ID NO: 47
MQFGCNVAEAFGLRRSGVVLLTDQSLRSMPLSQQKKVEIILDGMGRGSQAAQGLPSPITSLAFIRDSHHFLFLA
VDEDQCLGILKGGIKHLFMLDSQNETHEMDAMCCLDFYTHETVQRRGIGTRLFRAMELHTHISAQGWAFDRPSP
KLLAFLSKVYDMHDFKAQPNNFLMLDASIRLWGAEFKQYRRSKKHYIPDAYLLPETRESEYLGEAELTKRTLIR
KSTAVIPQTKTTQSEDAPARALTADELLSKRSVVLPTASRTPSLPDQPQSVAAAYMNKRIEGAGPSFEQYMRDH
YGAKSLIVPSEIQTSLNHSKDSVSQEDMIQRQRQLDRMAFTLAREANARGSIHNTVGRGVICGRRG
Hs, Homo sapiens gi|10435053|dbj|BAB14472.1|
SEQ ID NO: 48
MEFPFDVDALFPERITVLDQHLRPPARRPGTTTPARVDLQQQIMTIIDELGKASAKAQNLSAPITSASRMQSNR
HVVYILKDSSARPAGKGAIIGFIKVGYKKLFVLDDREAHNEVEPLCILDFYIHESVQRHGHGRELFQYMLQKER
VEPHQLAIDRPSQKLLKFLNKHYNLETTVPQVNNFVIFEGFFAHQHRPPAPSLRATRHSRAAAVDPTPAAPARK
LPPKRAEGDIKPYSSSDREFLKVAVEPPWPLNRAPRRATPPAHPPPRSSSLGNSPERGPLRPFVPEQELLRSLR
LCPPHPTARLLLAADPGGSPAQRRRTR
M, Micromonas sp. RCC299gi|255083454|ref|XP_002504713.1|
SEQ ID NO: 49
MQHPRGVAGLTPGGVSVWTRDAIVRLPPDEYRAISALIDEAGARSARAQGLPAPITSTDRLLEDQRLYLACSDA
TRPRGGPSVLVGILKVGPKRLFVAKPSGGMEEMEPCCVLDFYVHESSQRGGWGSLLFDAFLQREDRHPARLAYD
RPSPKLVAFMAKHHNLRAFAKQNNNFVVFDEYWSEE
Pp, Physcomitrella patens subsp. patens gi|168025715|ref|XP_001765379.1|
SEQ ID NO: 50
MVEFVCSIPSLKTFTEVPKITAWNSYQLMNLMRGKDGEEMQIIINYMGELSAIAQGLRGPITSVDRLLQSFQKI
YLLTSSVPGNSGHILAQGILKVGQKHLFIRRTPNAPLVEISPLCVLDFYAGTGAYVIQVHARAGEEEAMSAWVR
LSSFPSPVPVPVEITSNYDKPSNKLLSFMSKYYNLRSYNDQANQFVVYNDYFVDAKNEAQKEKENRDKELNGTP
DIGTNPRKDEVLHDSQANLQVIDVDDQCSSNRSDISRLTTAEARPPNLHVKEPPKQPLFRGGRKKIWQSKQGYS
EKPPNSADTLHTTDARSSLQSPTKATLSLKDAYRQGLNDQGISLHARKSSNSLSSEPKRCQENHGANNLHEPGV
TLLYSVIGEEERLKIQSMCTPSLAQKMNVKHPTRANHKDPKPIKPRPNMGSPVTRPTRQLTQKDPPPSCPLWAD
AKELSESQHWQERNPVDYKRTGKTMPVRNTRAEKIRSSINRMGDARNLAKALVHLSEQSGWFHCQWINDICDTW
CVPHYLIKMHSTVNWSSQSWKNRHAYTELAVTGLLANGHYSQMGSA
Pt, Paramecium tetraurelia gi|145495487|ref|XP_001433736.1|
SEQ ID NO: 51
MQFQFPLQKALQTSQNGISVISASNSRRNCYLDEVIDRMGEASAIAQQLKQIITTASKFYGSDQRIYLKADGKN
CLGLLKVGKKNLFYRDYSGSIKEMQPLCVLDFYVHESVQRMGVGKELFEEMLKSEQIKPEKLAYDRPSQKLIGF
LNKHYNLNQYVPQNNNFVIFNQYFGQGTQPIAQGRSQKYSRNSQIDQLDIMVQQISQNKTNQQQLPQQKLGYQM
STPWAIDNHTNIYNNINSQRTNVYKIN
Sj, Schistosoma japonicum gi|56756064|gb|AAW26210.1|SJCHGC00609
SEQ ID NO: 52
MDFRAGLENVLQQEVTVIHGEETRKLCIANNKYLNGKDADTFRNLAVLLDHLGERSAKAQKLPKPVTSFIKFRN
SDQSIFLLSDIPVKKFVILIYMFFRVLGFLKVGRKRLFVHDSKGVCVECIPLCILDFYIHESHQRKGYGKKLFD
FMLKTENIQPSYLAIDLPSMKMIQFLHKHYHLINPIYSPNNFVVYSEFFNNLNNSNYSIVQSKLTTTNCFLKSN
SSRIHQNKHNHSIVSNNNNNNNNNIIIIIIIQNITHHNNQLTIEQ
Tb, Trypanosoma brucei TREU927 gi|72386619|ref|XP_843734.1|
SEQ ID NO: 53
MTHNVMCDDVLPQLNLPDGVTRWNANLLEEERRLRNSDGHADRIILTINTLGKRSKEAQSLNTILTSVPRLREN
RDARLYLLCHGGRGVGILKIGVKRLFVVPPSHAGLMEIEPVCVLDFFVDTSNQRQGYGKILFEHMLAFERLSPG
DVAIDRPSVKFLAFLRKHYGLVEYTPQSNNFVVFHKYFERHQQQRRGVGGSGRSGYQHCNETTTQQLGTQSGLL
EDINQTHPAPSYALRGVVMGHTGPPLDLTNVTQQQKPYHQPFATGRKTSYELQYERYLQSQNCRPTGNAGYGGG
NGPASSAEVRATNCQARRRTSPTRSGVPYNIINGSTGS
Tc, Trypanosoma cruzi strain CL Brener gi|71661627|ref|XP_817832.1|
SEQ ID NO: 54
MSSTSQVALLPKLSLPDGVTVWDGTALEYERRCNNIDEHAVHLMQTINILGIRSKEAQCLNTVLTSVARLRENR
QARVYLLCQDGYGVGILKMGVKKLFVTHPSYSSLVEIDPLCVLDFFVDTSFQRKGFGKTLFDAMLLNEGLNPGE
VAIDRPSVKFLAFLRKYYGLVEYTPQSNNFVVFHRYFDKWQPQRGKGHRGGNAVPTRSIVRPQNCLRVYPEYQS
TTGPNDNFEEDATHRTPPPPLPPPLVPQGSVNSPGPGKKTAYELQYEEYLREQAYRRRQGGDPRLQPVPNPVSS
SEIAAASCGARRRMSPTRSGVQYNIISGTPEH
Tcas, Tribolium castaneum gi|189235028|ref|XP_972158.2|
SEQ ID NO: 55
MEFKFAVNDVFKQPIVKIGNNLLPPGFTGDRRAFWDVVGKVSEVVNAMGEASAAAQGLTKPITTAERLRNSEHS
VYLLIDQNAGNGRGAVTGMLKTGMKGLYVFDRNGQHYQVSPPCVLDFYVHESRQRTGLGKRLFEHMLQTESIEP
VKMAIDRPSEKFLGFLNKHYSLNNPVKQMNNYVVFDGFFPGAQDKNGTVESHSSHSNTPVGKKQSANGLQTSTY
ASPLGRYGAPRPPCSMGQIIHNQTSTIAKTPEPTGETQVMDEKVNSSPKLERPRSLSIQPSDEPTEDNNQNEND
VESEVLEEVEIIENLTIENGADVVDTKNEKIATPEAKTPGKRTPSHLTEQGFFDLKFYHNKLW
Tt, Tetrahymena thermophila gi|118354427|ref|XP_001010476.1|
SEQ ID NO: 56
MFKNQLLSLSLRIQKLRLCRQGNQLKVLQKSINQLLFKTQSIDQILQYLQQKKKTHQSILENQNQHIHSIGLVS
QLTNQKINKQKNQKAMEFNFIINKLVQLDQQGLGVYIPRASRSKVSSQQEQQLGQVLNTMGERSAIAQGLKQVI
TNYDKVQGTDQRVYIVAEGRTCQGFLKVGQKNLFYRDMMGNIKEIKPLCVLDFYVHESCQRQGYGKLLFEYMIQ
CEQTSPEKLAYDRPSPKLIAFLKKHYNLVKYIAQNNNFVVFDQYFRSDASSQNKQNQNTRSYSQPYSDYSSQIP
TNYPQQQQQQSNSKSYPYKQENNIDLMQHSSSRNNKEFLNAGRAILSKEEIYKKDNLSQQNIENTLNNINNSQY
STKSQQQQQYQKDYQLDKYENNWGADKNIKKPPFPGQDRQLDKIQQKIQQTERELDVVNQQIIKQRQNLSQDPL
TQNHRAQNVYNTNQFGTSPWAQTGFNYYSTSSSNYGNHYTYYKK
Xl, Xenopus laevis gi|148237085|ref|NP_001089986.1|
SEQ ID NO: 57
MEFEFDVHKIFLEPITKLDNNLIPPRRPLISSSEAQKQIMTVIDEIGKASAKAQRLPASITSASRMQANKHHLY
ILKDCTPKTAGRGAVIGFLKVGYKKLFILDQKGSHIEAEPLCILDFYIHESLQRHGFGKELFSFMLRNEQVDVQ
HLAIDRPSEKFLSFLRKHFNLWSTIPQVNNFVVFEGFFRDRKASVKKTPAKRTEGEIKPYSLTDRDFLKQEEGL
PWPLSQAQINLNRASSLGSSPTRACSRPPPGEEDFVKSLRNCRPHSLQRAASSEQEDHSQRRRTSEMNLSRGLL
AQKNGYSRYLSPPPPLLTQGPYAAAQIKEQQSRTDSSAQEGRTQDRPNGSNSQHQNDLISSKQHVQDLHMELAA
GRTMSDLKEGQNATKSPWCDHPSYTVLGTVLNAAWVKKKQELRSTRPW
Xt, Xenopus (Silurana) tropicalis gi|187937169|ref|NP_001120781.1|
SEQ ID NO: 58
MEFDFDVHKIFLEPITKLDSSLIPSRRPLIASSEAQKQIMTVIDEIGKASAKAQRLPAPITSASRMQTNKHHLY
ILKDCTPKTAGRGAVIGFLKVGCKKLFVLDQKGSHIEAEPLCILDFYIHETLQRHGFGKELFTFMLKNEQVDVH
HLAIDRPSEKFLSFLRKHFNLWSTIPQVNNFVVFDGFFRDWKASVKKTPAKRTEGEIKPYSLTDRDFLKQEEGL
PWPFSQSQLNLNRASSLGSSPTRACSRHSPGEEDFVKSLRNCRPHSLHRTANSEQEDHSQRRRTSSLNRPQSIH
H
Chlamydomonas reinhardtii; Cre07.g345150
SEQ ID NO: 59
MEFDFASLAFGGEQHISFWDSKRIATLKPDDQELLTKLLDVFGKKSAVAQGLGAPVTDIYRLRSTDQRLYLYMY
RESRKTVVLGGLKVGSKRLFVRTGTADLREIEPVCVLDFYVHESCQRQGVGKALFEHFLMAEGHDPATLAYDRP
SPKLLAFLRKHYGLEQYVPQSNNYVVFDRYWELCPPGSRQPHRGPGSMPPGASGQLQGRNASRGSVASISTPSG
HGGHGPRATPLMGMMPPGSGAGGYPPHWTPSALGSPSGGHPGGPPAPIGNSPSFGRRWASNNFGQGPPQAAPPY
PAPGTAGGPAFSIPPAGGDAQGGIVDALDAFARQQQQQGSGASSPQRGGAGAPWEGGAAGPAGTGASGFGGGAG
TWPPQGAPELPPAGAPSGMQPPGSRGAYSYRPPWATDEAAGPSGGSASGGVDAGPGAGPGAAGYAGFNSPPPPM
RTPSKDRFGMGPGNGGPAPVQRSPMHSILGGGGGYDAVAGGRSGAAQTRALQQQLLSMQAGDGAVAAAAAGGPV
LGGGYGGGGGHPGSYQQQQQHAPPSLYSGAPPAHGAGQGGDEDGGRLPGMMGHGSLALGMLGAAPSGRHGAGSG
GHAGGPGGGPPGGTSAGTPPAAGGHSAAKLMAVKQRSGAGAADCLVW

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1.-25. (canceled)

26. A method for identifying a compound that modulates α-tubulin acetyltransferase (αTAT) activity of an MEC17 polypeptide, the method comprising:
contacting a tubulin substrate with a MEC 17 polypeptide and a candidate modulator compound; and
detecting a change in the acetylation level or acetylation rate of the tubulin substrate;
wherein a reduced acetylation level or acetylation rate of the tubulin substrate in the presence of the candidate compound, compared to the acetylation level or acetylation rate in the absence of the candidate compound, is indicative that the candidate compound is an inhibitory compound that inhibits α-TAT activity; and
wherein an increased acetylation level or acetylation rate of the tubulin substrate in the presence of the candidate compound, compared to the acetylation level or acetylation rate in the absence of the candidate compound, is indicative that the candidate compound is an enhancer compound that stimulates α-TAT activity.

27. The method of claim 26, wherein contacting the tubulin substrate with the MEC17 polypeptide and a candidate modulator compound occurs within a cell.

28. The method of claim 27, wherein the cell is a genetically modified cell that overexpresses MEC17 polypeptide.

29. The method of claim 27, wherein the cell is a ciliated cell, and wherein detecting the acetylation level or acetylation rate of the tubulin substrate comprises detecting a change in cell growth rate, cell motility, phagocytosis, or ciliary beat frequency.

30. The method of claim 27, wherein the cell is a Tetrahymena cell.

31. The method of claim 27, wherein the tubulin substrate is selected from the group consisting of an axoneme, a microtubule, and an α-tubulin.

32. The method of claim 26, wherein the contacting step occurs in vitro and comprises contacting the tubulin substrate with a MEC17 polypeptide, a candidate modulator compound, and acetyl coenzyme A (acetyl CoA).

33. The method of claim 32, wherein the detecting step comprises detecting the acetylation level or acetylation rate of the tubulin substrate, wherein a reduced acetylation level or acetylation rate of the tubulin substrate in the presence of acetyl CoA and the candidate compound, compared to the acetylation level or acetylation rate in the presence of acetyl CoA and in the absence of the candidate compound, is indicative that the candidate compound is an inhibitory compound that inhibits α-TAT activity; and
wherein an increased acetylation level or acetylation rate of the tubulin substrate in the presence of acetyl CoA and the candidate compound, compared to the acetylation level or acetylation rate in the presence of acetyl CoA and in the absence of the candidate compound, is indicative that the candidate compound is an enhancer compound that stimulates α-TAT activity

34. The method of claim 32, wherein the tubulin substrate is selected from the group consisting of an axoneme, a microtubule, and an α-tubulin.

35. The method of claim 34, wherein the tubulin substrate is a microtubule that was polymerized from isolated tubulin in vitro.

36. The method of claim 34, wherein the tubulin substrate comprises axonemes or microtubules comprising non-acetylated α-tubulin.

37. The method of claim 36, wherein the nonacetylated microtubules or nonacetylated axonemes have been chemically or enzymatically deacetylated in vitro.

38. The method of claim 34, wherein the tubulin substrate comprises microtubules or axonemes lacking detectable α-tubulin acetylation at position K40.

39. The method of claim 32, wherein the tubulin substrate is obtained from a eukaryotic cell that lacks MEC-17 activity or from a cell that contains α-tubulin that is naturally non-acetylated at position K40 or has low levels of acetylation at position K40.

40. The method of claim 32, wherein the tubulin substrate is obtained from a cell that has been genetically engineered to either fail to express or to express reduced amounts of a MEC-17 polypeptide.

41. The method of claim 40, wherein the tubulin substrate is obtained from a MEC-17 gene knockout Tetrahymena cell.

42. The method of claim 32, further comprising, prior to the contacting step, isolating the MEC17 polypeptide from a cell that has been genetically engineered to overexpress MEC17 polypeptide.

43. The method of claim 26, wherein the detecting step comprises detecting acetylation at position K40 of α-tubulin using an antibody specific for acetylated lysine, an antibody specific for acetyl-K40 on α-tubulin, or detectably labeled acetyl-CoA.
